SEQUENCE LISTING (335 sequences)

SEQ ID NO: 1 - DNA, 26 bp, Artificial Sequence
  Description of Artificial Sequence Synthetic oligonucleotide
gcggaaaacttcctgcacatgatgtt

SEQ ID NO: 2 - DNA, 26 bp, Artificial Sequence
  Description of Artificial Sequence Synthetic oligonucleotide
gcggaaaacttcctccacatgatgtt

SEQ ID NO: 3 - DNA, 23 bp, Artificial Sequence
  Description of Artificial Sequence Synthetic oligonucleotide
agctcagcagactgctgacgagg

SEQ ID NO: 4 - DNA, 22 bp, Artificial Sequence
  Description of Artificial Sequence Synthetic oligonucleotide
agctcagcagaccgctgacgag

SEQ ID NO: 5 - DNA, 25 bp, Artificial Sequence
  Description of Artificial Sequence Synthetic oligonucleotide
aagaggacggccgccgggtgacgcc

SEQ ID NO: 6 - DNA, 26 bp, Artificial Sequence
  Description of Artificial Sequence Synthetic oligonucleotide
aagaggacggccgccaggtgacgccg

SEQ ID NO: 7 - DNA, 22 bp, Artificial Sequence
  Description of Artificial Sequence Synthetic oligonucleotide
gacaagatgcgcctcgacgacc

SEQ ID NO: 8 - DNA, 23 bp, Artificial Sequence
  Description of Artificial Sequence Synthetic oligonucleotide
gacaagatgcgtctcgacgaccg

SEQ ID NO: 9 - DNA, 22 bp, Artificial Sequence
  Description of Artificial Sequence Synthetic oligonucleotide
agccgacctacgcgccgggcag

SEQ ID NO: 10 - DNA, 23 bp, Artificial Sequence
  Description of Artificial Sequence Synthetic oligonucleotide
cagccgacctatgcgccgggcag

SEQ ID NO: 11 - DNA, 23 bp, Artificial Sequence
  Description of Artificial Sequence Synthetic oligonucleotide
ccgttcgaacggctcatggagca

SEQ ID NO: 12 - DNA, 24 bp, Artificial Sequence
  Description of Artificial Sequence Synthetic oligonucleotide
gccgttcgaacgactcatggagca

SEQ ID NO: 13 - DNA, 22 bp, Artificial Sequence
  Description of Artificial Sequence Synthetic oligonucleotide
tggagcagcaagtgttcccggc

SEQ ID NO: 14 - DNA, 22 bp, Artificial Sequence
  Description of Artificial Sequence Synthetic oligonucleotide
tggagcagcaactgttcccggc

SEQ ID NO: 15 - DNA, 24 bp, Artificial Sequence
  Description of Artificial Sequence Synthetic oligonucleotide
gaacaagaccggttccaccaacgg

SEQ ID NO: 16 - DNA, 23 bp, Artificial Sequence
  Description of Artificial Sequence Synthetic oligonucleotide
aacaagaccggctccaccaacgg

SEQ ID NO: 17 - DNA, 22 bp, Artificial Sequence
  Description of Artificial Sequence Synthetic oligonucleotide
gcgacctgggcctggtgatcct

SEQ ID NO: 18 - DNA, 22 bp, Artificial Sequence
  Description of Artificial Sequence Synthetic oligonucleotide
gcgacctgggactggtgatcct

SEQ ID NO: 19 - DNA, 24 bp, Artificial Sequence
  Description of Artificial Sequence Synthetic oligonucleotide
gccgaccaactgaactccaactcg

SEQ ID NO: 20 - DNA, 23 bp, Artificial Sequence
  Description of Artificial Sequence Synthetic oligonucleotide
gtcgctgaacggcacctacttca

SEQ ID NO: 21 - DNA, 22 bp, Artificial Sequence
  Description of Artificial Sequence Synthetic oligonucleotide
cagcctgcggtcatgtcctcgg

SEQ ID NO: 22 - DNA, 24 bp, Artificial Sequence
  Description of Artificial Sequence Synthetic oligonucleotide
cgccagtttgagaacggagtcacc

SEQ ID NO: 23 - DNA, 24 bp, Artificial Sequence
  Description of Artificial Sequence Synthetic oligonucleotide
gcgcgatcttctccacttcatcgg

SEQ ID NO: 24 - DNA, 24 bp, Artificial Sequence
  Description of Artificial Sequence Synthetic oligonucleotide
gcctccgcgattgaacatcgtgat

SEQ ID NO: 25 - DNA, 24 bp, Artificial Sequence
  Description of Artificial Sequence Synthetic oligonucleotide
gtagccggagtcgagcggaatcat

SEQ ID NO: 26 - DNA, 24 bp, Artificial Sequence
  Description of Artificial Sequence Synthetic oligonucleotide
gtgagcatggaatcggcagtcgtt

SEQ ID NO: 27 - DNA, 24 bp, Artificial Sequence
  Description of Artificial Sequence Synthetic oligonucleotide
cgaggagtttcggacccgctttga

SEQ ID NO: 28 - DNA, 24 bp, Artificial Sequence
  Description of Artificial Sequence Synthetic oligonucleotide
aataggaccggcagaacgggcatt

SEQ ID NO: 29 - DNA, 24 bp, Artificial Sequence
  Description of Artificial Sequence Synthetic oligonucleotide
gcgccttctcctctttgcagatgt

SEQ ID NO: 30 - DNA, 24 bp, Artificial Sequence
  Description of Artificial Sequence Synthetic oligonucleotide
cagtatggtacggacacgaagcgc

SEQ ID NO: 31 - DNA, 24 bp, Artificial Sequence
  Description of Artificial Sequence Synthetic oligonucleotide
gcatcattgcgcgtcacatctggt

SEQ ID NO: 32 - DNA, 24 bp, Artificial Sequence
  Description of Artificial Sequence Synthetic oligonucleotide
tctgaactgcggctatcacctgga

SEQ ID NO: 33 - DNA, 24 bp, Artificial Sequence
  Description of Artificial Sequence Synthetic oligonucleotide
aattgatggcttctcaggcgcagg

SEQ ID NO: 34 - DNA, 25 bp, Artificial Sequence
  Description of Artificial Sequence Synthetic oligonucleotide
agtcatgggactgaatacggcgact

SEQ ID NO: 35 - DNA, 24 bp, Artificial Sequence
  Description of Artificial Sequence Synthetic oligonucleotide
ttctcggtgtcgagggattctcgg

SEQ ID NO: 36 - DNA, 24 bp, Artificial Sequence
  Description of Artificial Sequence Synthetic oligonucleotide
tggtagctctcgacgtactggctg

SEQ ID NO: 37 - DNA, 24 bp, Artificial Sequence
  Description of Artificial Sequence Synthetic oligonucleotide
cccgttgctcataacccgttcctg

SEQ ID NO: 38 - DNA, 24 bp, Artificial Sequence
  Description of Artificial Sequence Synthetic oligonucleotide
agggcattctcaggtggactcagg

SEQ ID NO: 39 - DNA, 24 bp, Artificial Sequence
  Description of Artificial Sequence Synthetic oligonucleotide
acctgtgtcgctggagggtatgtt

SEQ ID NO: 40 - DNA, 24 bp, Artificial Sequence
  Description of Artificial Sequence Synthetic oligonucleotide
agcgtccctgaccaacctcatcag

SEQ ID NO: 41 - DNA, 24 bp, Artificial Sequence
  Description of Artificial Sequence Synthetic oligonucleotide
cgccaacaattcgccattacagcg

SEQ ID NO: 42 - DNA, 24 bp, Artificial Sequence
  Description of Artificial Sequence Synthetic oligonucleotide
tccaacaggcaggagtacagggtg

SEQ ID NO: 43 - DNA, 24 bp, Artificial Sequence
  Description of Artificial Sequence Synthetic oligonucleotide
cgctgcacatacaggtccgttctc

SEQ ID NO: 44 - DNA, 25 bp, Artificial Sequence
  Description of Artificial Sequence Synthetic oligonucleotide
agcccagcaattgcgtgtttctccg

SEQ ID NO: 45 - DNA, 24 bp, Artificial Sequence
  Description of Artificial Sequence Synthetic oligonucleotide
agcccagcaactgcgtgtttctcc

SEQ ID NO: 46 - DNA, 19 bp, Artificial Sequence
  Description of Artificial Sequence Synthetic oligonucleotide
gctgctggcggcggtgtgc

SEQ ID NO: 47 - DNA, 21 bp, Artificial Sequence
  Description of Artificial Sequence Synthetic oligonucleotide
tgctgctggcagcggtgtgct

SEQ ID NO: 48 - DNA, 27 bp, Artificial Sequence
  Description of Artificial Sequence Synthetic oligonucleotide
cagaaagctcagcagactgctgacgag

SEQ ID NO: 49 - DNA, 25 bp, Artificial Sequence
  Description of Artificial Sequence Synthetic oligonucleotide
gaaagctcagcagaccgctgacgag

SEQ ID NO: 50 - DNA, 19 bp, Artificial Sequence
  Description of Artificial Sequence Synthetic oligonucleotide
acggccgccgggtgacgcc

SEQ ID NO: 51 - DNA, 20 bp, Artificial Sequence
  Description of Artificial Sequence Synthetic oligonucleotide
acggccgccaggtgacgccg

SEQ ID NO: 52 - DNA, 19 bp, Artificial Sequence
  Description of Artificial Sequence Synthetic oligonucleotide
gccgacctacgcgccgggc

SEQ ID NO: 53 - DNA, 21 bp, Artificial Sequence
  Description of Artificial Sequence Synthetic oligonucleotide
agccgacctatgcgccgggca

SEQ ID NO: 54 - DNA, 24 bp, Artificial Sequence
  Description of Artificial Sequence Synthetic oligonucleotide
gttcgaacggctcatggagcagca

SEQ ID NO: 55 - DNA, 26 bp, Artificial Sequence
  Description of Artificial Sequence Synthetic oligonucleotide
gttcgaacgactcatggagcagcaag

SEQ ID NO: 56 - DNA, 20 bp, Artificial Sequence
  Description of Artificial Sequence Synthetic oligonucleotide
cagcccagtcaggacgcgca

SEQ ID NO: 57 - DNA, 24 bp, Artificial Sequence
  Description of Artificial Sequence Synthetic oligonucleotide
agtgacgtgcgtttcagcagtccc

SEQ ID NO: 58 - DNA, 24 bp, Artificial Sequence
  Description of Artificial Sequence Synthetic oligonucleotide
gtgtcacggcccatgtctagcagc

SEQ ID NO: 59 - DNA, 23 bp, Artificial Sequence
  Description of Artificial Sequence Synthetic oligonucleotide
cgaagtctgaggtgtggacccgc

SEQ ID NO: 60 - DNA, 24 bp, Artificial Sequence
  Description of Artificial Sequence Synthetic oligonucleotide
cgctggagggtatgttccgcaagg

SEQ ID NO: 61 - DNA, 24 bp, Artificial Sequence
  Description of Artificial Sequence Synthetic oligonucleotide
cgtactcagcttctccacccagcg

SEQ ID NO: 62 - DNA, 24 bp, Artificial Sequence
  Description of Artificial Sequence Synthetic oligonucleotide
cctggacctctccaaggttcgcct

SEQ ID NO: 63 - DNA, 24 bp, Artificial Sequence
  Description of Artificial Sequence Synthetic oligonucleotide
gccattccgacgaccaaacaaggc

SEQ ID NO: 64 - DNA, 43 bp, Artificial Sequence
  Description of Artificial Sequence Synthetic oligonucleotide
gtctccctggagcctgcgaaagtggctcggttgcgtagccgac

SEQ ID NO: 65 - DNA, 89 bp, Artificial Sequence
  Description of Artificial Sequence Synthetic oligonucleotide
atgttgtatttttcttgcggtatgaagatgggtggttgggtcggatataggtacttctctctattttctttaattgctcttatctatgg

SEQ ID NO: 66 - DNA, 54 bp, Artificial Sequence
  Description of Artificial Sequence Synthetic oligonucleotide
gacctcgacccccgagggcttcatggcgtgtcgcgaactcgcatggcaacaggc

SEQ ID NO: 67 - DNA, 100 bp, Artificial Sequence
  Description of Artificial Sequence Synthetic oligonucleotide
tggtcagccgagtaaccggcagttgtcgccaggtctggagaatcccgccattagcttgattcgacggaactatagcgactttggtccaactctggcccag

SEQ ID NO: 68 - DNA, 77 bp, Artificial Sequence
  Description of Artificial Sequence Synthetic oligonucleotide
atgggcaagagagtggttgtattgctatggctgctattcacatcaatgtcagcccacgccatcgataaaaaagtcaa

SEQ ID NO: 69 - DNA, 73 bp, Artificial Sequence
  Description of Artificial Sequence Synthetic oligonucleotide
cggctcggacatggccaattgggtcagcaagcaacgcgccggaggcatgcctgggttcgccaggggcggtgcc

SEQ ID NO: 70 - DNA, 50 bp, Artificial Sequence
  Description of Artificial Sequence Synthetic oligonucleotide
gttcctggaacgagggtgatggctgggaatacgtggaggcgccacagccg

SEQ ID NO: 71 - DNA, 44 bp, Artificial Sequence
  Description of Artificial Sequence Synthetic oligonucleotide
atgttcgtacatgacaagcgactgcagtacaccgtcagggtcgc

SEQ ID NO: 72 - DNA, 23 bp, Artificial Sequence
  Description of Artificial Sequence Synthetic oligonucleotide
gaagcccagcaattgcgtgtttc

SEQ ID NO: 73 - DNA, 23 bp, Artificial Sequence
  Description of Artificial Sequence Synthetic oligonucleotide
gaagcccagcaactgcgtgtttc

SEQ ID NO: 74 - DNA, 25 bp, Artificial Sequence
  Description of Artificial Sequence Synthetic oligonucleotide
agcccagcaattgcgtgtttctccg

SEQ ID NO: 75 - DNA, 24 bp, Artificial Sequence
  Description of Artificial Sequence Synthetic oligonucleotide
agcccagcaactgcgtgtttctcc

SEQ ID NO: 76 - DNA, 23 bp, Artificial Sequence
  Description of Artificial Sequence Synthetic oligonucleotide
ggtgctgcagggtgtttcgccgg

SEQ ID NO: 77 - DNA, 23 bp, Artificial Sequence
  Description of Artificial Sequence Synthetic oligonucleotide
ggtgctgcagggcgtttcgccgg

SEQ ID NO: 78 - DNA, 22 bp, Artificial Sequence
  Description of Artificial Sequence Synthetic oligonucleotide
caagatcgccgcagcggtcaac

SEQ ID NO: 79 - DNA, 22 bp, Artificial Sequence
  Description of Artificial Sequence Synthetic oligonucleotide
caagatcgccgctgcggtcaac

SEQ ID NO: 80 - DNA, 23 bp, Artificial Sequence
  Description of Artificial Sequence Synthetic oligonucleotide
tgctgctggcggcggtgtgctat

SEQ ID NO: 81 - DNA, 23 bp, Artificial Sequence
  Description of Artificial Sequence Synthetic oligonucleotide
tgctgctggcagcggtgtgctat

SEQ ID NO: 82 - DNA, 19 bp, Artificial Sequence
  Description of Artificial Sequence Synthetic oligonucleotide
gctgctggcggcggtgtgc

SEQ ID NO: 83 - DNA, 21 bp, Artificial Sequence
  Description of Artificial Sequence Synthetic oligonucleotide
tgctgctggcagcggtgtgct

SEQ ID NO: 84 - DNA, 25 bp, Artificial Sequence
  Description of Artificial Sequence Synthetic oligonucleotide
cctcgccctgttcccaccgctctgg

SEQ ID NO: 85 - DNA, 24 bp, Artificial Sequence
  Description of Artificial Sequence Synthetic oligonucleotide
ctcgccctgttcccgccgctctgg

SEQ ID NO: 86 - DNA, 24 bp, Artificial Sequence
  Description of Artificial Sequence Synthetic oligonucleotide
tcgagcaactggcagagaaatccg

SEQ ID NO: 87 - DNA, 23 bp, Artificial Sequence
  Description of Artificial Sequence Synthetic oligonucleotide
cgagcaactggcggagaaatccg

SEQ ID NO: 88 - DNA, 26 bp, Artificial Sequence
  Description of Artificial Sequence Synthetic oligonucleotide
gcggaaaacttcctgcacatgatgtt

SEQ ID NO: 89 - DNA, 26 bp, Artificial Sequence
  Description of Artificial Sequence Synthetic oligonucleotide
gcggaaaacttcctccacatgatgtt

SEQ ID NO: 90 - DNA, 23 bp, Artificial Sequence
  Description of Artificial Sequence Synthetic oligonucleotide
agctcagcagactgctgacgagg

SEQ ID NO: 91 - DNA, 22 bp, Artificial Sequence
  Description of Artificial Sequence Synthetic oligonucleotide
agctcagcagaccgctgacgag

SEQ ID NO: 92 - DNA, 27 bp, Artificial Sequence
  Description of Artificial Sequence Synthetic oligonucleotide
cagaaagctcagcagactgctgacgag

SEQ ID NO: 93 - DNA, 25 bp, Artificial Sequence
  Description of Artificial Sequence Synthetic oligonucleotide
gaaagctcagcagaccgctgacgag

SEQ ID NO: 94 - DNA, 19 bp, Artificial Sequence
  Description of Artificial Sequence Synthetic oligonucleotide
acggccgccgggtgacgcc

SEQ ID NO: 95 - DNA, 20 bp, Artificial Sequence
  Description of Artificial Sequence Synthetic oligonucleotide
acggccgccaggtgacgccg

SEQ ID NO: 96 - DNA, 22 bp, Artificial Sequence
  Description of Artificial Sequence Synthetic oligonucleotide
gacaagatgcgcctcgacgacc

SEQ ID NO: 97 - DNA, 23 bp, Artificial Sequence
  Description of Artificial Sequence Synthetic oligonucleotide
gacaagatgcgtctcgacgaccg

SEQ ID NO: 98 - DNA, 22 bp, Artificial Sequence
  Description of Artificial Sequence Synthetic oligonucleotide
agccgacctacgcgccgggcag

SEQ ID NO: 99 - DNA, 23 bp, Artificial Sequence
  Description of Artificial Sequence Synthetic oligonucleotide
cagccgacctatgcgccgggcag

SEQ ID NO: 100 - DNA, 19 bp, Artificial Sequence
  Description of Artificial Sequence Synthetic oligonucleotide
gccgacctacgcgccgggc

SEQ ID NO: 101 - DNA, 21 bp, Artificial Sequence
  Description of Artificial Sequence Synthetic oligonucleotide
agccgacctatgcgccgggca

SEQ ID NO: 102 - DNA, 24 bp, Artificial Sequence
  Description of Artificial Sequence Synthetic oligonucleotide
gttcgaacggctcatggagcagca

SEQ ID NO: 103 - DNA, 26 bp, Artificial Sequence
  Description of Artificial Sequence Synthetic oligonucleotide
gttcgaacgactcatggagcagcaag

SEQ ID NO: 104 - DNA, 22 bp, Artificial Sequence
  Description of Artificial Sequence Synthetic oligonucleotide
tggagcagcaagtgttcccggc

SEQ ID NO: 105 - DNA, 22 bp, Artificial Sequence
  Description of Artificial Sequence Synthetic oligonucleotide
tggagcagcaactgttcccggc

SEQ ID NO: 106 - DNA, 24 bp, Artificial Sequence
  Description of Artificial Sequence Synthetic oligonucleotide
gaacaagaccggttccaccaacgg

SEQ ID NO: 107 - DNA, 23 bp, Artificial Sequence
  Description of Artificial Sequence Synthetic oligonucleotide
aacaagaccggctccaccaacgg

SEQ ID NO: 108 - DNA, 22 bp, Artificial Sequence
  Description of Artificial Sequence Synthetic oligonucleotide
gcgacctgggcctggtgatcct

SEQ ID NO: 109 - DNA, 22 bp, Artificial Sequence
  Description of Artificial Sequence Synthetic oligonucleotide
gcgacctgggactggtgatcct

SEQ ID NO: 110 - DNA, 21 bp, Artificial Sequence
  Description of Artificial Sequence Synthetic oligonucleotide
gtgctgcagggtgtttcgccg

SEQ ID NO: 111 - DNA, 19 bp, Artificial Sequence
  Description of Artificial Sequence Synthetic oligonucleotide
gctgcagggcgtttcgccg

SEQ ID NO: 112 - DNA, 28 bp, Artificial Sequence
  Description of Artificial Sequence Synthetic oligonucleotide
gctcagcagactgctgacgaggctaacg

SEQ ID NO: 113 - DNA, 27 bp, Artificial Sequence
  Description of Artificial Sequence Synthetic oligonucleotide
gctcagcagaccgctgacgaggctaac

SEQ ID NO: 114 - DNA, 19 bp, Artificial Sequence
  Description of Artificial Sequence Synthetic oligonucleotide
cgacctacgcgccgggcag

SEQ ID NO: 115 - DNA, 20 bp, Artificial Sequence
  Description of Artificial Sequence Synthetic oligonucleotide
cgacctatgcgccgggcagc

SEQ ID NO: 116 - DNA, 23 bp, Artificial Sequence
  Description of Artificial Sequence Synthetic oligonucleotide
cgttcgaacggctcatggagcag

SEQ ID NO: 117 - DNA, 24 bp, Artificial Sequence
  Description of Artificial Sequence Synthetic oligonucleotide
cgttcgaacgactcatggagcagc

SEQ ID NO: 118 - DNA, 21 bp, Artificial Sequence
  Description of Artificial Sequence Synthetic oligonucleotide
cgacctgggcctggtgatcct

SEQ ID NO: 119 - DNA, 24 bp, Artificial Sequence
  Description of Artificial Sequence Synthetic oligonucleotide
gcgacctgggactggtgatcctgg

SEQ ID NO: 120 - DNA, 24 bp, Artificial Sequence
  Description of Artificial Sequence Synthetic oligonucleotide
gcgcgatcttctccacttcatcgg

SEQ ID NO: 121 - DNA, 24 bp, Artificial Sequence
  Description of Artificial Sequence Synthetic oligonucleotide
cgaggagtttcggacccgctttga

SEQ ID NO: 122 - DNA, 24 bp, Artificial Sequence
  Description of Artificial Sequence Synthetic oligonucleotide
aataggaccggcagaacgggcatt

SEQ ID NO: 123 - DNA, 23 bp, Artificial Sequence
  Description of Artificial Sequence Synthetic oligonucleotide
cgaagtctgaggtgtggacccgc

SEQ ID NO: 124 - DNA, 24 bp, Artificial Sequence
  Description of Artificial Sequence Synthetic oligonucleotide
gcatcattgcgcgtcacatctggt

SEQ ID NO: 125 - DNA, 24 bp, Artificial Sequence
  Description of Artificial Sequence Synthetic oligonucleotide
tctgaactgcggctatcacctgga

SEQ ID NO: 126 - DNA, 25 bp, Artificial Sequence
  Description of Artificial Sequence Synthetic oligonucleotide
agtcatgggactgaatacggcgact

SEQ ID NO: 127 - DNA, 24 bp, Artificial Sequence
  Description of Artificial Sequence Synthetic oligonucleotide
ttctcggtgtcgagggattctcgg

SEQ ID NO: 128 - DNA, 24 bp, Artificial Sequence
  Description of Artificial Sequence Synthetic oligonucleotide
tggtagctctcgacgtactggctg

SEQ ID NO: 129 - DNA, 24 bp, Artificial Sequence
  Description of Artificial Sequence Synthetic oligonucleotide
cccgttgctcataacccgttcctg

SEQ ID NO: 130 - DNA, 24 bp, Artificial Sequence
  Description of Artificial Sequence Synthetic oligonucleotide
agggcattctcaggtggactcagg

SEQ ID NO: 131 - DNA, 24 bp, Artificial Sequence
  Description of Artificial Sequence Synthetic oligonucleotide
gcgccttctcctctttgcagatgt

SEQ ID NO: 132 - DNA, 24 bp, Artificial Sequence
  Description of Artificial Sequence Synthetic oligonucleotide
cagtatggtacggacacgaagcgc

SEQ ID NO: 133 - DNA, 24 bp, Artificial Sequence
  Description of Artificial Sequence Synthetic oligonucleotide
tccaacaggcaggagtacagggtg

SEQ ID NO: 134 - DNA, 24 bp, Artificial Sequence
  Description of Artificial Sequence Synthetic oligonucleotide
cgctgcacatacaggtccgttctc

SEQ ID NO: 135 - DNA, 25 bp, Artificial Sequence
  Description of Artificial Sequence Synthetic oligonucleotide
caagatcgccgcagcggtcaacgac

SEQ ID NO: 136 - DNA, 25 bp, Artificial Sequence
  Description of Artificial Sequence Synthetic oligonucleotide
caagatcgccgctgcggtcaacgac

SEQ ID NO: 137 - DNA, 26 bp, Artificial Sequence
  Description of Artificial Sequence Synthetic oligonucleotide
cagttgtcgccaggtctggagaatcc

SEQ ID NO: 138 - DNA, 23 bp, Artificial Sequence
  Description of Artificial Sequence Synthetic oligonucleotide
cacatcaatgtcagcccacgcca

SEQ ID NO: 139 - DNA, 22 bp, Artificial Sequence
  Description of Artificial Sequence Synthetic oligonucleotide
ctggagcctgcgaaagtggctc

SEQ ID NO: 140 - DNA, 24 bp, Artificial Sequence
  Description of Artificial Sequence Synthetic oligonucleotide
acgagggtgatggctgggaatacg

SEQ ID NO: 141 - DNA, 23 bp, Artificial Sequence
  Description of Artificial Sequence Synthetic oligonucleotide
gccaattgggtcagcaagcaacg

SEQ ID NO: 142 - DNA, 21 bp, Artificial Sequence
  Description of Artificial Sequence Synthetic oligonucleotide
cgtgtcgcgaactcgcatggc

SEQ ID NO: 143 - DNA, 24 bp, Artificial Sequence
  Description of Artificial Sequence Synthetic oligonucleotide
cctgaatccgaccattcgcgagtc

SEQ ID NO: 144 - DNA, 25 bp, Artificial Sequence
  Description of Artificial Sequence Synthetic oligonucleotide
tcggactgtactcctacgaagcagc

SEQ ID NO: 145 - DNA, 26 bp, Artificial Sequence
  Description of Artificial Sequence Synthetic oligonucleotide
ccaatccctatcgctggaaccgtacc

SEQ ID NO: 146 - DNA, 22 bp, Artificial Sequence
  Description of Artificial Sequence Synthetic oligonucleotide
gctcgggactcgcatttcgtcc

SEQ ID NO: 147 - DNA, 24 bp, Artificial Sequence
  Description of Artificial Sequence Synthetic oligonucleotide
gcgttattgctcggtctctcctcg

SEQ ID NO: 148 - DNA, 23 bp, Artificial Sequence
  Description of Artificial Sequence Synthetic oligonucleotide
gaccgcaagcagaaacggcatgc

SEQ ID NO: 149 - DNA, 26 bp, Artificial Sequence
  Description of Artificial Sequence Synthetic oligonucleotide
ccatggtcggaacaggcacgatatgc

SEQ ID NO: 150 - DNA, 28 bp, Artificial Sequence
  Description of Artificial Sequence Synthetic oligonucleotide
ccactcgatcatgttgagcatcggctcc

SEQ ID NO: 151 - DNA, 24 bp, Artificial Sequence
  Description of Artificial Sequence Synthetic oligonucleotide
ggttagtcccttctgcccgcatcg

SEQ ID NO: 152 - DNA, 24 bp, Artificial Sequence
  Description of Artificial Sequence Synthetic oligonucleotide
gtgtcacggcccatgtctagcagc

SEQ ID NO: 153 - DNA, 24 bp, Artificial Sequence
  Description of Artificial Sequence Synthetic oligonucleotide
gtgagcatggaatcggcagtcgtt

SEQ ID NO: 154 - DNA, 24 bp, Artificial Sequence
  Description of Artificial Sequence Synthetic oligonucleotide
acctgtgtcgctggagggtatgtt

SEQ ID NO: 155 - DNA, 24 bp, Artificial Sequence
  Description of Artificial Sequence Synthetic oligonucleotide
cgctggagggtatgttccgcaagg

SEQ ID NO: 156 - DNA, 24 bp, Artificial Sequence
  Description of Artificial Sequence Synthetic oligonucleotide
cgtactcagcttctccacccagcg

SEQ ID NO: 157 - DNA, 24 bp, Artificial Sequence
  Description of Artificial Sequence Synthetic oligonucleotide
cctggacctctccaaggttcgcct

SEQ ID NO: 158 - DNA, 24 bp, Artificial Sequence
  Description of Artificial Sequence Synthetic oligonucleotide
gccattccgacgaccaaacaaggc

SEQ ID NO: 159 - DNA, 22 bp, Artificial Sequence
  Description of Artificial Sequence Synthetic oligonucleotide
aggccatgggctagccggatgc

SEQ ID NO: 160 - DNA, 23 bp, Artificial Sequence
  Description of Artificial Sequence Synthetic oligonucleotide
cgaagcgtagggtcttcgtagcc

SEQ ID NO: 161 - DNA, 24 bp, Artificial Sequence
  Description of Artificial Sequence Synthetic oligonucleotide
tgcgaggaccagaaaccttgatgg

SEQ ID NO: 162 - DNA, 26 bp, Artificial Sequence
  Description of Artificial Sequence Synthetic oligonucleotide
cggtatgaagatgggtggttgggtcg

SEQ ID NO: 163 - DNA, 24 bp, Artificial Sequence
  Description of Artificial Sequence Synthetic oligonucleotide
tgcataggagtcatgccgacagca

SEQ ID NO: 164 - DNA, 23 bp, Artificial Sequence
  Description of Artificial Sequence Synthetic oligonucleotide
gcctgcctacttgttcccaacgc

SEQ ID NO: 165 - DNA, 23 bp, Artificial Sequence
  Description of Artificial Sequence Synthetic oligonucleotide
ggctgtattgcccgccattctcc

SEQ ID NO: 166 - DNA, 25 bp, Artificial Sequence
  Description of Artificial Sequence Synthetic oligonucleotide
cgacagacagaaagggttcttgcgc

SEQ ID NO: 167 - DNA, 26 bp, Artificial Sequence
  Description of Artificial Sequence Synthetic oligonucleotide
caccatgcaaatgctcgatggactgc

SEQ ID NO: 168 - DNA, 25 bp, Artificial Sequence
  Description of Artificial Sequence Synthetic oligonucleotide
gcaggcgtccaagttggagctctcc

SEQ ID NO: 169 - DNA, 22 bp, Artificial Sequence
  Description of Artificial Sequence Synthetic oligonucleotide
ggaacacaacgtggggcgtgac

SEQ ID NO: 170 - DNA, 23 bp, Artificial Sequence
  Description of Artificial Sequence Synthetic oligonucleotide
ccagttggcaccaccatgcttgc

SEQ ID NO: 171 - DNA, 20 bp, Artificial Sequence
  Description of Artificial Sequence Synthetic oligonucleotide
cagcccagtcaggacgcgca

SEQ ID NO: 172 - DNA, 24 bp, Artificial Sequence
  Description of Artificial Sequence Synthetic oligonucleotide
cgccagtttgagaacggagtcacc

SEQ ID NO: 173 - DNA, 24 bp, Artificial Sequence
  Description of Artificial Sequence Synthetic oligonucleotide
agtgacgtgcgtttcagcagtccc

SEQ ID NO: 174 - DNA, 24 bp, Artificial Sequence
  Description of Artificial Sequence Synthetic oligonucleotide
gccgaccaactgaactccaactcg

SEQ ID NO: 175 - DNA, 23 bp, Artificial Sequence
  Description of Artificial Sequence Synthetic oligonucleotide
gtcgctgaacggcacctacttca

SEQ ID NO: 176 - DNA, 21 bp, Artificial Sequence
  Description of Artificial Sequence Synthetic primer
acgcggatgtcctggatttgg

SEQ ID NO: 177 - DNA, 21 bp, Artificial Sequence
  Description of Artificial Sequence Synthetic primer
ctgaagaaggggcgctacgcg

SEQ ID NO: 178 - DNA, 21 bp, Artificial Sequence
  Description of Artificial Sequence Synthetic primer
gcgtaccgggcaaggtgatag

SEQ ID NO: 179 - DNA, 21 bp, Artificial Sequence
  Description of Artificial Sequence Synthetic primer
ctcggtgaaacatcgggaggg

SEQ ID NO: 180 - DNA, 21 bp, Artificial Sequence
  Description of Artificial Sequence Synthetic primer
tcatccagcaagccattgcgc

SEQ ID NO: 181 - DNA, 20 bp, Artificial Sequence
  Description of Artificial Sequence Synthetic primer
ggagtcgctttccgccatcg

SEQ ID NO: 182 - DNA, 21 bp, Artificial Sequence
  Description of Artificial Sequence Synthetic primer
tggagtcgctttccgccatcg

SEQ ID NO: 183 - DNA, 21 bp, Artificial Sequence
  Description of Artificial Sequence Synthetic primer
aagggcgtttcacgctgacgc

SEQ ID NO: 184 - DNA, 20 bp, Artificial Sequence
  Description of Artificial Sequence Synthetic primer
atccggaagggcgtttcacg

SEQ ID NO: 185 - DNA, 21 bp, Artificial Sequence
  Description of Artificial Sequence Synthetic primer
tccacacctcagacttcggcg

SEQ ID NO: 186 - DNA, 21 bp, Artificial Sequence
  Description of Artificial Sequence Synthetic primer
tattgacgacctaccgcgcgc

SEQ ID NO: 187 - DNA, 20 bp, Artificial Sequence
  Description of Artificial Sequence Synthetic primer
gcaactgatgttcgcccagc

SEQ ID NO: 188 - DNA, 21 bp, Artificial Sequence
  Description of Artificial Sequence Synthetic primer
cgcaactgatgttcgcccagc

SEQ ID NO: 189 - DNA, 21 bp, Artificial Sequence
  Description of Artificial Sequence Synthetic primer
acacgcaactgatgttcgccc

SEQ ID NO: 190 - DNA, 20 bp, Artificial Sequence
  Description of Artificial Sequence Synthetic primer
tgtcccggctcagttcaacg

SEQ ID NO: 191 - DNA, 20 bp, Artificial Sequence
  Description of Artificial Sequence Synthetic primer
aacaccttggcgtttgtccc

SEQ ID NO: 192 - DNA, 21 bp, Artificial Sequence
  Description of Artificial Sequence Synthetic primer
gcaacaccttggcgtttgtcc

SEQ ID NO: 193 - DNA, 20 bp, Artificial Sequence
  Description of Artificial Sequence Synthetic primer
tcaagctcgttgtggaccgc

SEQ ID NO: 194 - DNA, 21 bp, Artificial Sequence
  Description of Artificial Sequence Synthetic primer
gttacgacggcgtgctgtcgg

SEQ ID NO: 195 - DNA, 21 bp, Artificial Sequence
  Description of Artificial Sequence Synthetic primer
acgcaacgtattcggcgaccc

SEQ ID NO: 196 - DNA, 20 bp, Artificial Sequence
  Description of Artificial Sequence Synthetic primer
cgcaacgtattcggcgaccc

SEQ ID NO: 197 - DNA, 21 bp, Artificial Sequence
  Description of Artificial Sequence Synthetic primer
agctgatggtatcgccgtcgc

SEQ ID NO: 198 - DNA, 20 bp, Artificial Sequence
  Description of Artificial Sequence Synthetic primer
ctagtgatcgcaccggagcc

SEQ ID NO: 199 - DNA, 20 bp, Artificial Sequence
  Description of Artificial Sequence Synthetic primer
agcctcgacaccggttctcg

SEQ ID NO: 200 - DNA, 20 bp, Artificial Sequence
  Description of Artificial Sequence Synthetic primer
tcgttcatccccaggcttcg

SEQ ID NO: 201 - DNA, 21 bp, Artificial Sequence
  Description of Artificial Sequence Synthetic primer
accatctcgttcatccccagg

SEQ ID NO: 202 - DNA, 21 bp, Artificial Sequence
  Description of Artificial Sequence Synthetic primer
ttctgagcccaggactgctcg

SEQ ID NO: 203 - DNA, 21 bp, Artificial Sequence
  Description of Artificial Sequence Synthetic primer
tcgacgcgacggttctgagcc

SEQ ID NO: 204 - DNA, 20 bp, Artificial Sequence
  Description of Artificial Sequence Synthetic primer
tgacgttctcgccggtagcg

SEQ ID NO: 205 - DNA, 21 bp, Artificial Sequence
  Description of Artificial Sequence Synthetic primer
cagtagcggtaccggtctgcg

SEQ ID NO: 206 - DNA, 20 bp, Artificial Sequence
  Description of Artificial Sequence Synthetic primer
cagtagcggtaccggtctgc

SEQ ID NO: 207 - DNA, 21 bp, Artificial Sequence
  Description of Artificial Sequence Synthetic primer
ttcctcgccggcatagtaggc

SEQ ID NO: 208 - DNA, 21 bp, Artificial Sequence
  Description of Artificial Sequence Synthetic primer
ggggtcgagacgtgtacatgg

SEQ ID NO: 209 - DNA, 21 bp, Artificial Sequence
  Description of Artificial Sequence Synthetic primer
cgaggacgaggcatcttccgg

SEQ ID NO: 210 - DNA, 20 bp, Artificial Sequence
  Description of Artificial Sequence Synthetic primer
gcaggtagcaggtttccagg

SEQ ID NO: 211 - DNA, 21 bp, Artificial Sequence
  Description of Artificial Sequence Synthetic primer
aactgttccttctgcgcggcg

SEQ ID NO: 212 - DNA, 21 bp, Artificial Sequence
  Description of Artificial Sequence Synthetic primer
tgatcggcttggtctcgcagg

SEQ ID NO: 213 - DNA, 20 bp, Artificial Sequence
  Description of Artificial Sequence Synthetic primer
gctgatcggcttggtctcgc

SEQ ID NO: 214 - DNA, 21 bp, Artificial Sequence
  Description of Artificial Sequence Synthetic primer
gaggcgttctgctcgtggtcg

SEQ ID NO: 215 - DNA, 20 bp, Artificial Sequence
  Description of Artificial Sequence Synthetic primer
tttttccagcatgcgcaggg

SEQ ID NO: 216 - DNA, 21 bp, Artificial Sequence
  Description of Artificial Sequence Synthetic primer
gctggctttttccagcatgcg

SEQ ID NO: 217 - DNA, 21 bp, Artificial Sequence
  Description of Artificial Sequence Synthetic primer
ttgcggctggctttttccagc

SEQ ID NO: 218 - DNA, 20 bp, Artificial Sequence
  Description of Artificial Sequence Synthetic primer
ttgggatagttgcggttggc

SEQ ID NO: 219 - DNA, 20 bp, Artificial Sequence
  Description of Artificial Sequence Synthetic primer
cgtaggcgatcttcacccgc

SEQ ID NO: 220 - DNA, 21 bp, Artificial Sequence
  Description of Artificial Sequence Synthetic primer
tggcgtaggcgatcttcaccc

SEQ ID NO: 221 - DNA, 20 bp, Artificial Sequence
  Description of Artificial Sequence Synthetic primer
ggcgagatagccgaacaggc

SEQ ID NO: 222 - DNA, 21 bp, Artificial Sequence
  Description of Artificial Sequence Synthetic primer
gcggcgagatagccgaacagg

SEQ ID NO: 223 - DNA, 20 bp, Artificial Sequence
  Description of Artificial Sequence Synthetic primer
cacttgctgctccatgagcc

SEQ ID NO: 224 - DNA, 20 bp, Artificial Sequence
  Description of Artificial Sequence Synthetic primer
gaggtcgagcaggctgatgc

SEQ ID NO: 225 - DNA, 21 bp, Artificial Sequence
  Description of Artificial Sequence Synthetic primer
taggtcgcgaggtcgagcagg

SEQ ID NO: 226 - DNA, 20 bp, Artificial Sequence
  Description of Artificial Sequence Synthetic primer
gtccttctgcaccgagtcgg

SEQ ID NO: 227 - DNA, 21 bp, Artificial Sequence
  Description of Artificial Sequence Synthetic primer
cgcatcttgtcctgggtcagg

SEQ ID NO: 228 - DNA, 21 bp, Artificial Sequence
  Description of Artificial Sequence Synthetic primer
tcgtcgaggcgcatcttgtcc

SEQ ID NO: 229 - DNA, 21 bp, Artificial Sequence
  Description of Artificial Sequence Synthetic primer
acgtcgaggtgggtctgttcg

SEQ ID NO: 230 - DNA, 20 bp, Artificial Sequence
  Description of Artificial Sequence Synthetic primer
gtagccttcggcatccagcg

SEQ ID NO: 231 - DNA, 21 bp, Artificial Sequence
  Description of Artificial Sequence Synthetic primer
tcggcattgggatagttgcgg

SEQ ID NO: 232 - DNA, 21 bp, Artificial Sequence
  Description of Artificial Sequence Synthetic primer
cctcctgtctcatgccgatgc

SEQ ID NO: 233 - DNA, 21 bp, Artificial Sequence
  Description of Artificial Sequence Synthetic primer
gcattcgccacggaaggaagg

SEQ ID NO: 234 - DNA, 21 bp, Artificial Sequence
  Description of Artificial Sequence Synthetic primer
gaaggcatcatggcattcgcc

SEQ ID NO: 235 - DNA, 22 bp, Artificial Sequence
  Description of Artificial Sequence Synthetic primer
gtcatggggtttcccagagacc

SEQ ID NO: 236 - DNA, 21 bp, Artificial Sequence
  Description of Artificial Sequence Synthetic primer
gatcgcgatgtcgacggtgcc

SEQ ID NO: 237 - DNA, 21 bp, Artificial Sequence
  Description of Artificial Sequence Synthetic primer
cgatcgcgatgtcgacggtgc

SEQ ID NO: 238 - DNA, 20 bp, Artificial Sequence
  Description of Artificial Sequence Synthetic primer
tgccgatcgcgatgtcgacg

SEQ ID NO: 239 - DNA, 21 bp, Artificial Sequence
  Description of Artificial Sequence Synthetic primer
gacgaatacccagctgcgtgg

SEQ ID NO: 240 - DNA, 21 bp, Artificial Sequence
  Description of Artificial Sequence Synthetic primer
gcagacgaatacccagctgcg

SEQ ID NO: 241 - DNA, 20 bp, Artificial Sequence
  Description of Artificial Sequence Synthetic primer
cgcgacgtcgtgacgtcagc

SEQ ID NO: 242 - DNA, 21 bp, Artificial Sequence
  Description of Artificial Sequence Synthetic primer
actttcggctcttcgggctgg

SEQ ID NO: 243 - DNA, 21 bp, Artificial Sequence
  Description of Artificial Sequence Synthetic primer
aggtagagactcgggggaacc

SEQ ID NO: 244 - DNA, 21 bp, Artificial Sequence
  Description of Artificial Sequence Synthetic primer
tcgttttcggtcatggccagg

SEQ ID NO: 245 - DNA, 21 bp, Artificial Sequence
  Description of Artificial Sequence Synthetic primer
ttccgcgacgaacatccgtgg

SEQ ID NO: 246 - DNA, 21 bp, Artificial Sequence
  Description of Artificial Sequence Synthetic primer
cgcttccgcgacgaacatccg

SEQ ID NO: 247 - DNA, 22 bp, Artificial Sequence
  Description of Artificial Sequence Synthetic primer
ggatcgcttccgatagggcagc

SEQ ID NO: 248 - DNA, 20 bp, Artificial Sequence
  Description of Artificial Sequence Synthetic primer
agaggcatgggtctgtaccg

SEQ ID NO: 249 - DNA, 20 bp, Artificial Sequence
  Description of Artificial Sequence Synthetic primer
tctgtcaatcccctttgggg

SEQ ID NO: 250 - DNA, 21 bp, Artificial Sequence
  Description of Artificial Sequence Synthetic primer
agcccctttctgtcaatcccc

SEQ ID NO: 251 - DNA, 21 bp, Artificial Sequence
  Description of Artificial Sequence Synthetic primer
ggcttcctaccgaaggtcagg

SEQ ID NO: 252 - DNA, 20 bp, Artificial Sequence
  Description of Artificial Sequence Synthetic primer
tgagggcttcctaccgaagg

SEQ ID NO: 253 - DNA, 21 bp, Artificial Sequence
  Description of Artificial Sequence Synthetic primer
ttcaaggtcatgggcaatgcc

SEQ ID NO: 254 - DNA, 21 bp, Artificial Sequence
  Description of Artificial Sequence Synthetic primer
agtcccttcaaggtcatgggc

SEQ ID NO: 255 - DNA, 21 bp, Artificial Sequence
  Description of Artificial Sequence Synthetic primer
gccgactgagctgtagctcgg

SEQ ID NO: 256 - DNA, 21 bp, Artificial Sequence
  Description of Artificial Sequence Synthetic primer
ggccgactgagctgtagctcg

SEQ ID NO: 257 - DNA, 20 bp, Artificial Sequence
  Description of Artificial Sequence Synthetic primer
accagactggtcaatggtgg

SEQ ID NO: 258 - DNA, 22 bp, Artificial Sequence
  Description of Artificial Sequence Synthetic primer
cccgtgtttccgtagaccttgc

SEQ ID NO: 259 - DNA, 20 bp, Artificial Sequence
  Description of Artificial Sequence Synthetic primer
agcagttacccacagcatgg

SEQ ID NO: 260 - DNA, 21 bp, Artificial Sequence
  Description of Artificial Sequence Synthetic primer
cagcagttacccacagcatgg

SEQ ID NO: 261 - DNA, 21 bp, Artificial Sequence
  Description of Artificial Sequence Synthetic primer
ctacactccaaccgctggtcc

SEQ ID NO: 262 - DNA, 21 bp, Artificial Sequence
  Description of Artificial Sequence Synthetic primer
gacctacactccaaccgctgg

SEQ ID NO: 263 - DNA, 20 bp, Artificial Sequence
  Description of Artificial Sequence Synthetic primer
ttcccttgctgccgagaagc

SEQ ID NO: 264 - DNA, 21 bp, Artificial Sequence
  Description of Artificial Sequence Synthetic primer
taataggcgagcctgccgtcc

SEQ ID NO: 265 - DNA, 20 bp, Artificial Sequence
  Description of Artificial Sequence Synthetic primer
tccacgccgagggacgtgcc

SEQ ID NO: 266 - DNA, 22 bp, Artificial Sequence
  Description of Artificial Sequence Synthetic primer
gctccacgccgagggacgtgcc

SEQ ID NO: 267 - DNA, 20 bp, Artificial Sequence
  Description of Artificial Sequence Synthetic primer
cgcggtgctggttgcgctgc

SEQ ID NO: 268 - DNA, 21 bp, Artificial Sequence
  Description of Artificial Sequence Synthetic primer
ccaatgcccagggccagcgga

SEQ ID NO: 269 - DNA, 20 bp, Artificial Sequence
  Description of Artificial Sequence Synthetic primer
cgctggcagttccgctggcc

SEQ ID NO: 270 - DNA, 22 bp, Artificial Sequence
  Description of Artificial Sequence Synthetic primer
cagggtcgccagctcgctcgcc

SEQ ID NO: 271 - DNA, 20 bp, Artificial Sequence
  Description of Artificial Sequence Synthetic primer
agggtcgccagctcgctcgc

SEQ ID NO: 272 - DNA, 22 bp, Artificial Sequence
  Description of Artificial Sequence Synthetic primer
agtgatctgccgcggccctgcc

SEQ ID NO: 273 - DNA, 20 bp, Artificial Sequence
  Description of Artificial Sequence Synthetic primer
gtgatctgccgcggccctgc

SEQ ID NO: 274 - DNA, 21 bp, Artificial Sequence
  Description of Artificial Sequence Synthetic primer
gttccacaggcgctgcggcgc

SEQ ID NO: 275 - DNA, 20 bp, Artificial Sequence
  Description of Artificial Sequence Synthetic primer
gttccacaggcgctgcggcg

SEQ ID NO: 276 - DNA, 20 bp, Artificial Sequence
  Description of Artificial Sequence Synthetic primer
caaagcccctggtcgcgcgg

SEQ ID NO: 277 - DNA, 22 bp, Artificial Sequence
  Description of Artificial Sequence Synthetic primer
gcagcttttccaccgccggcgg

SEQ ID NO: 278 - DNA, 21 bp, Artificial Sequence
  Description of Artificial Sequence Synthetic primer
aaactgccccgccccccatcc

SEQ ID NO: 279 - DNA, 21 bp, Artificial Sequence
  Description of Artificial Sequence Synthetic primer
ggaaaaactgccccgcccccc

SEQ ID NO: 280 - DNA, 21 bp, Artificial Sequence
  Description of Artificial Sequence Synthetic primer
acgctcgcagcgcctcacgcg

SEQ ID NO: 281 - DNA, 21 bp, Artificial Sequence
  Description of Artificial Sequence Synthetic primer
ggcctggctgcgaacgctcgc

SEQ ID NO: 282 - DNA, 21 bp, Artificial Sequence
  Description of Artificial Sequence Synthetic primer
ttcctgggccagagttggacc

SEQ ID NO: 283 - DNA, 21 bp, Artificial Sequence
  Description of Artificial Sequence Synthetic primer
agcttaaggccgtggcactcg

SEQ ID NO: 284 - DNA, 20 bp, Artificial Sequence
  Description of Artificial Sequence Synthetic primer
ccggagaattcgcgtccacc

SEQ ID NO: 285 - DNA, 21 bp, Artificial Sequence
  Description of Artificial Sequence Synthetic primer
tgctgacgatgaagccccagc

SEQ ID NO: 286 - DNA, 21 bp, Artificial Sequence
  Description of Artificial Sequence Synthetic primer
aggaggccgatgacaacaccc

SEQ ID NO: 287 - DNA, 21 bp, Artificial Sequence
  Description of Artificial Sequence Synthetic primer
tgccgattccatgctcacgcc

SEQ ID NO: 288 - DNA, 21 bp, Artificial Sequence
  Description of Artificial Sequence Synthetic primer
acgacgtcaccgtcgagaccg

SEQ ID NO: 289 - DNA, 21 bp, Artificial Sequence
  Description of Artificial Sequence Synthetic primer
accgcctttctggtgagctgg

SEQ ID NO: 290 - DNA, 21 bp, Artificial Sequence
  Description of Artificial Sequence Synthetic primer
agccaagacggttgttcgcgg

SEQ ID NO: 291 - DNA, 21 bp, Artificial Sequence
  Description of Artificial Sequence Synthetic primer
tcaatgacgccgagttggcgc

SEQ ID NO: 292 - DNA, 20 bp, Artificial Sequence
  Description of Artificial Sequence Synthetic primer
ctcggacaggttcacgctgg

SEQ ID NO: 293 - DNA, 21 bp, Artificial Sequence
  Description of Artificial Sequence Synthetic primer
gccattcgctgcaacacctcc

SEQ ID NO: 294 - DNA, 20 bp, Artificial Sequence
  Description of Artificial Sequence Synthetic primer
gcgcgcgttcgagaaacagg

SEQ ID NO: 295 - DNA, 21 bp, Artificial Sequence
  Description of Artificial Sequence Synthetic primer
cggaggttgaaaagctggccc

SEQ ID NO: 296 - DNA, 22 bp, Artificial Sequence
  Description of Artificial Sequence Synthetic primer
atgccatcgttgaaggcaccgc

SEQ ID NO: 297 - DNA, 20 bp, Artificial Sequence
  Description of Artificial Sequence Synthetic primer
tgccatcgttgaaggcaccg

SEQ ID NO: 298 - DNA, 21 bp, Artificial Sequence
  Description of Artificial Sequence Synthetic primer
tctggcggaatcaggtaggcc

SEQ ID NO: 299 - DNA, 20 bp, Artificial Sequence
  Description of Artificial Sequence Synthetic primer
cttccggggagaaaccaccg

SEQ ID NO: 300 - DNA, 20 bp, Artificial Sequence
  Description of Artificial Sequence Synthetic primer
acctccagcaccgacacacc

SEQ ID NO: 301 - DNA, 21 bp, Artificial Sequence
  Description of Artificial Sequence Synthetic primer
atccgatccacctccagcacc

SEQ ID NO: 302 - DNA, 21 bp, Artificial Sequence
  Description of Artificial Sequence Synthetic primer
cgttcaggtcgtagaccgcgc

SEQ ID NO: 303 - DNA, 22 bp, Artificial Sequence
  Description of Artificial Sequence Synthetic primer
gcgataccaactgtcctgcggc

SEQ ID NO: 304 - DNA, 22 bp, Artificial Sequence
  Description of Artificial Sequence Synthetic primer
tgccgaaggtgaatggcttgcc

SEQ ID NO: 305 - DNA, 20 bp, Artificial Sequence
  Description of Artificial Sequence Synthetic primer
cctgatggtccgatcccagc

SEQ ID NO: 306 - DNA, 22 bp, Artificial Sequence
  Description of Artificial Sequence Synthetic primer
gccgagggtcaagaaccactgg

SEQ ID NO: 307 - DNA, 21 bp, Artificial Sequence
  Description of Artificial Sequence Synthetic primer
tcttggcccagtcatagcggc

SEQ ID NO: 308 - DNA, 22 bp, Artificial Sequence
  Description of Artificial Sequence Synthetic primer
taaccccaaggcccattggagg

SEQ ID NO: 309 - DNA, 22 bp, Artificial Sequence
  Description of Artificial Sequence Synthetic primer
gccaccgccttcgaataacccc

SEQ ID NO: 310 - DNA, 20 bp, Artificial Sequence
  Description of Artificial Sequence Synthetic primer
aattgctcgagggatgcggc

SEQ ID NO: 311 - DNA, 20 bp, Artificial Sequence
  Description of Artificial Sequence Synthetic primer
ggtcgaaacggatgcgcagg

SEQ ID NO: 312 - DNA, 22 bp, Artificial Sequence
  Description of Artificial Sequence Synthetic primer
gccccgcgtcattttcacgtcg

SEQ ID NO: 313 - DNA, 20 bp, Artificial Sequence
  Description of Artificial Sequence Synthetic primer
aatgctctgggcaacgagcc

SEQ ID NO: 314 - DNA, 20 bp, Artificial Sequence
  Description of Artificial Sequence Synthetic primer
ctacccagcttgggcgtagc

SEQ ID NO: 315 - DNA, 21 bp, Artificial Sequence
  Description of Artificial Sequence Synthetic primer
aagcgatagccgtgctcctgc

SEQ ID NO: 316 - DNA, 21 bp, Artificial Sequence
  Description of Artificial Sequence Synthetic primer
ccggctatatccgcggctacc

SEQ ID NO: 317 - DNA, 22 bp, Artificial Sequence
  Description of Artificial Sequence Synthetic primer
attggcgctgctgtttacgccc

SEQ ID NO: 318 - DNA, 21 bp, Artificial Sequence
  Description of Artificial Sequence Synthetic primer
ggtggcgtcgggtttttctgc

SEQ ID NO: 319 - DNA, 22 bp, Artificial Sequence
  Description of Artificial Sequence Synthetic primer
aggtcgtagcggaaggtggtgg

SEQ ID NO: 320 - DNA, 21 bp, Artificial Sequence
  Description of Artificial Sequence Synthetic primer
atctgaaccgaggggatccgc

SEQ ID NO: 321 - DNA, 20 bp, Artificial Sequence
  Description of Artificial Sequence Synthetic primer
cccgggagtcattggtctgg

SEQ ID NO: 322 - DNA, 21 bp, Artificial Sequence
  Description of Artificial Sequence Synthetic primer
gcctgttggacccctttgacc

SEQ ID NO: 323 - DNA, 21 bp, Artificial Sequence
  Description of Artificial Sequence Synthetic primer
tactcctgcctgttggacccc

SEQ ID NO: 324 - DNA, 20 bp, Artificial Sequence
  Description of Artificial Sequence Synthetic primer
cgctcaagcgctatcccacc

SEQ ID NO: 325 - DNA, 20 bp, Artificial Sequence
  Description of Artificial Sequence Synthetic primer
cgccatcggcctgtacaacg

SEQ ID NO: 326 - DNA, 20 bp, Artificial Sequence
  Description of Artificial Sequence Synthetic primer
cggtagagagctgggttggc

SEQ ID NO: 327 - DNA, 21 bp, Artificial Sequence
  Description of Artificial Sequence Synthetic primer
aacctggagctagggcagagc

SEQ ID NO: 328 - DNA, 20 bp, Artificial Sequence
  Description of Artificial Sequence Synthetic primer
ggtgctcgacccaagcatcg

SEQ ID NO: 329 - DNA, 21 bp, Artificial Sequence
  Description of Artificial Sequence Synthetic primer
tccttgagttccttggcgcgg

SEQ ID NO: 330 - DNA, 20 bp, Artificial Sequence
  Description of Artificial Sequence Synthetic primer
caacacgcgactggcgatcc

SEQ ID NO: 331 - DNA, 21 bp, Artificial Sequence
  Description of Artificial Sequence Synthetic primer
tacatcatccgcaacggcggc

SEQ ID NO: 332 - DNA, 22 bp, Artificial Sequence
  Description of Artificial Sequence Synthetic primer
tattgacgacctaccgcgcgcc

SEQ ID NO: 333 - DNA, 20 bp, Artificial Sequence
  Description of Artificial Sequence Synthetic primer
caccaagaacccgctgctcg

SEQ ID NO: 334 - DNA, 21 bp, Artificial Sequence
  Description of Artificial Sequence Synthetic primer
atcgtggcaggatgtccaccg

SEQ ID NO: 335 - DNA, 22 bp, Artificial Sequence
  Description of Artificial Sequence Synthetic primer
taggcgggccttttgaaggtgc

(19) United States
(12) Patent Application Publication
(10) Pub. No.: US 2008/0026370 A1
(43) Pub. Date: Jan. 31, 2008

(54) Method For Geno-And Pathotyping Pseudomonas Aeruginosa

(75) Inventors: Gerd Wagner, Jena (DE); Lutz Wiehlmann, Hanover (DE); Burkhard Tuemmler, Hanover (DE)

Correspondence Address: STEPTOE & JOHNSON LLP, 1330 CONNECTICUT AVENUE, N.W., WASHINGTON, DC, 20036, US

(73) Assignee: Clonddag Chip Technology GmbH, Jena (DE)

(21) Appl. No.: 10/587,180

(22) PCT Filed: Jan. 26, 2005
(86) PCT No.: PCT/EP05/00751

§ 371 Date: May 30, 2007

(30) Foreign Application Priority Data
Jan. 26, 2004 (DE) 10 2004 003 860.0

(51) Int. Cl. C12Q 1/68; C07H 21/04; C40B 40/06

(52) U.S. Cl. 435/6; 506/16; 536/23.1

(57) ABSTRACT

The invention relates to a method for genotyping and pathotyping bacteria of the species Pseudomonas aeruginosa by means of hybridization assays on a biochip or microarray. The invention further relates to specific oligonucleotide probes, which can be employed within the scope of the detection method, as well as to biochips having such oligonucleotide probes.

The present invention relates to a method for genotyping and pathotyping bacteria of the species Pseudomonas aeruginosa by means of hybridization assays on a biochip or a microarray. The present invention further relates to specific oligonucleotide probes, which can be employed within the scope of the detection method, as well as to biochips having such oligonucleotide probes.

Pseudomonas aeruginosa is an ubiquitous environmental pathogen, which, being an opportunistic pathogen, causes high morbidity and mortality in patients being locally or systemically immunocompromised. By chronically colonizing the respiratory tracts in cystic fibrosis patients, Pseudomonas aeruginosa in addition crucially influences the course of disease. Due to the wide-ranging metabolic and adaptive capabilities of Pseudomonas aeruginosa, treatment of an infection is often very laborious and a total elimination of the bacterium is often not possible.

It has been shown that 70% of the infections with Pseudomonas aeruginosa in intensive care wards were caused by pathogens, which had already been detected in other patients before, in some cases even repeatedly or at intervals of several weeks. Beside all immediate consequences for the patients affected, such nosocomial infections indicate immense expenditure for the healthcare system. Due to the high rate of transmission within the clinic environment, there is thus a need for monitoring the incidence of infections and for avoiding spread and persistence of pathogens in the course of hygiene control.

In order to successfully avoid infections, recognizing the infection sources is of essential importance, wherein, in addition to detecting the pathogen, it often remains to be clarified, whether repeatedly isolated strains of the same species originate from the same clone or whether they have different origins. Related examinations are summarized under the term “pathogen typing”.

Reliable typing of strains of Pseudonionas aeruginosa has hitherto only been possible by means of molecular-biological methods, which are comparatively complex and expensive.

Thus, Pseudomonas aeruginosa isolates have up to now been typed with the aid of alternating field electrophoresis and assigned to the different subgroups. To this end, the genomic DNA of the respective strain was cut with restriction enzymes and then separated. Beside an expenditure of time of several weeks for each analysis, such an examination requires a high degree of previous experience and can be conducted in only few laboratories.

For reasons of costs arising from it, molecular-biological routine typing is thus not justified up to now. Therefore, there is a need for detection methods for Pseudomonas aeruginosa, which can be conducted by non-specialized molecular-biological routine laboratories in a cost-effective manner.

Thus the problem of the present invention is to provide a method for specifically detecting and for genotyping and pathotyping bacterial strains of the species Pseudomonas aeruginosa, which can be conducted with comparatively little technical effort and in a cost-effective manner. Another problem of the present invention is to provide a device for specifically detecting and for genotyping and pathotyping bacterial strains of the species Pseudomonas aeruginosa, which is characterized in that it can easily be handled and is compatible with devices conventionally used in molecular-biological laboratories, like for example table centrifuges and pipettes.

These and further problems underlying the present invention are solved by providing the subject matter specified in the patent claims.

Preferred embodiments are defined in the subclaims.

According to the present invention these problems are solved by providing a biochip or nucleic acid chip having oligonucleotide probes for specifically detecting bacterial strains of the species Pseudomonas aeruginosa.

The nucleic acid chip according to the present invention has the considerable advantage that, in this manner, Pseudomonas aeruginosa can be detected quickly and easily in a routine diagnostic laboratory within one day. In particular, the nucleic acid chip according to the present invention allows genotyping and pathotyping of Pseudomonas aeruginosa. The incidence of infections can thus be monitored and, in case a nosocomial spread of said pathogen is suspected, measures can immediately be taken in order to avoid propagation and persistence of Pseudomonas aeruginosa.

Within the scope of the present invention, a nucleic acid chip is to be understood as a support element, on which oligonucleotide probes are immobilized on predetermined regions. The predetermined regions on the support are also referred to as array elements in the following.

The use of a nucleic acid chip for specifically detecting Pseudomonas aeruginosa strains allows detection of the interaction reaction between the target nucleic acids present in the sample to be examined and oligonucleotide probes by means of conventional methods, for example by means of fluorescence detection or radiochemical methods. The use of absorption measurements has proven to be particularly advantageous, as said measurements can be conducted in a particularly cost-effective manner. Such an absorption measurement can be considerably improved and cheapened by means of using a reactive staining method, which occurs at those surface regions where an interaction reaction has taken place. Herein, inter alia, the precipitation of silver at target molecules labeled with gold nanobeads has proven its worth (see DE 100 33 334.6 and WO 02/02810). For detecting the silver precipitate, a device can be used, which employs one or more light-emitting diodes of arbitrary suitable emission wavelength as light source and, for example, a CCD camera for locally resolved detection of the interaction reaction on the predetermined regions of the chip.

For the description of the present invention, inter alia, the following definitions are used:

Within the scope of the present invention, a microarray or probe array is understood to denote a layout of molecular probes or a substance library on a support, wherein the position of each probe is determined separately. Preferably, the array comprises defined sites or predetermined regions, the so-called array elements, which are particularly preferably arranged in a specific pattern, wherein each array element normally contains only one species of probes. Herein, the layout of the molecules or probes on the support can be generated by means of covalent or non-covalent interactions. A position within the layout, i.e. within the array, is usually referred to as spot. Thus, the probe array forms the detection area.

Within the scope of the present invention, an array element or a predetermined region or a spot is understood to denote an area determined for depositing a molecular probe, or an area occupied by one or more defined molecular probes after deposition, on a surface; the entirety of all occupied array elements is the probe array or microarray.

Within the scope of the present invention, a probe or oligonucleotide probe is understood to denote a molecule used for detecting other molecules by means of a specific characteristic binding behavior or a specific reactivity. The probes arranged on the array can be any type of nucleic acids and/or analogs thereof, which can be coupled to solid surfaces and have a specific affinity. The oligonucleotides can comprise DNA molecules, RNA molecules, and/or analogs thereof, like for example artificial or modified nucleotides. The oligonucleotide probes can, for example, be oligonucleotides having a length of 10 to 100 bases, preferably 15 to 50 bases, and particularly preferably 20 to 30 bases, which are immobilized on the array surface.

Typically, according to the present invention, the oligonucleotide probes are single-stranded nucleic acid molecules or molecules of nucleic acid analogs, preferably single-stranded DNA molecules or RNA molecules having at least one sequence region, which is complementary to a sequence region of the target nucleic acids. Depending on the detection method and use, the oligonucleotide probes can be immobilized on a solid support substrate, for example in the form of a microarray. Furthermore, depending on the detection method, they can be labeled radioactively or non-radioactively, thus being detectable by means of a detection reaction conventional in the prior art.

Within the scope of the present invention, a target or a target nucleic acid is, in particular, understood to denote a nucleic acid present in the genome of Pseudomonas aeruginosa, which provides indications concerning the identity of a strain of the species Pseudomonas aeruginosa, which is present in the sample, of disease-associated genes, and/or the identity of the present flagella type. The target nucleic acids normally comprise sequences having a length of 40 to 10,000 bases, preferably of 60 to 2,000 bases, also preferably of 60 to 1,000 bases, particularly preferably of 60 to 500 bases, and most preferably of 60 to 150 bases. Optionally, their sequence contains the sequences of primers as well as the sequence regions of the template, which are defined by the primers. The target nucleic acids can, in particular, be single-stranded or double-stranded nucleic acid molecules, one or both strand/s of which is/are labeled after completion of a suitable treatment, as for example described in the prior art, so that they can be detected by means of detection methods conventional in the art. Particularly preferably, the target nucleic acids are nucleic acids having one base substitution in at least 30% of the population of Pseudomonas aeruginosa compared to the sequence of the genome of the reference strain PAO1 (see www.pseudomonas.com) of Pseudomonas aeruginosa; nucleic acids which are not present in all strains of the species Pseudomonas aeruginosa; nucleic acids which are present in pathogenicity islets in the genome of Pseudomonas aeruginosa; nucleic acids which are present in disease-associated genes like exoS and exoU; and nucleic acids which are contained in genes coding for flagella of Pseudomonas aeruginosa.

According to the present invention, the target sequence is understood to denote the sequence region of the target, which is detected by means of hybridization with the probe. According to the present invention, this is also referred to as said region being addressed by the probe.

Within the scope of the present invention, a substance library is understood to denote a multiplicity of different probe molecules, preferably at least 2 to 1,000,000 different molecules, particularly preferably at least 10 to 10,000 different molecules, and most preferably between 50 and 1,000 different molecules. In special embodiments, a substance library can also comprise only at least 50 or less or at least 30,000 different molecules. Preferably, the substance library is arranged in the form of an array on a support inside the reaction chamber of the device according to the present invention. Arranging the substances or probe molecules on the support is preferably performed in such a way that a specific, unambiguously identifiable site is assigned to each substance or each species of probe molecules and that each substance or each species of probe molecules is immobilized in such a way that it is separated from the others.

Within the scope of the present invention, a support element or support or substance library support is understood to denote a solid body, on which the probe array is assembled. The support, usually also referred to as substrate or matrix, can for example be a microscope slide or wafer or it can also consist of ceramic materials. The entirety of molecules deposited in array arrangements on the detection area or of the substance library deposited in array arrangements on the detection area and the support is also often referred to as “nucleic acid chip”, “chip”, “biochip”, “microarray”, “DNA chip”, “probe array” and the like.

Conventional nucleic acid chips or arrays or microarrays within the scope of the present invention comprise about 10 to 5,000, preferably 20 to 500, and particularly preferably 50 to 100 different species of oligonucleotide probes on a, preferably square, area of, for example, 1 mm to 4 mm×1 mm to 4 mm, preferably of 2 mm×2 mm or about 17.64 mm². In further embodiments, microarrays within the scope of the present invention comprise about 50 to about 80,000, preferably about 100 to about 65,000, particularly preferably about 1,000 to about 10,000 different species of probe molecules on an area of several mm² to several cm², preferably about 1 mm² to 10 cm², particularly preferably about 2 mm² to about 1 cm², and most preferably about 4 mm² to about 6.25 mm². A conventional microarray, for example, has 100 to 65,000 different species of probe molecules on an area of about 2.4 mm×about 2.4 mm. Further exemplary sizes of the areas of the microarray or the areas for synthesis of the biopolymers are about 1 to 10 mm×about 1 to 10 mm, preferably about 2.4 to 5 mm×about 2.4 to 5 mm, and most preferably about 3.5 to 4.5 mm×about 3.5 to 4.5 mm.

Within the scope of the present invention, a label is understood to denote a detectable unit, for example a fluorophore or an anchor group, whereto a detectable unit or a catalyst catalyzing the conversion of a soluble educt or substrate to form an insoluble product or a crystal nucleus can be coupled.

Within the scope of the present invention, an educt or substrate (in the sense of an enzymatic substrate) is understood to denote a molecule or a combination of molecules present in a state dissolved in the reaction medium, which is/are precipitated locally with the aid of a catalyst or a crystal nucleus and/or a converting agent. The converting agent can, for example, be a reducing agent like in silver precipitation or an oxidizing agent like in the production of a dye by means of enzymatic oxidation.

Within the scope of the present invention, the sample or sample solution or analyte is understood to denote the liquid to be analyzed containing the target molecules to be detected and, optionally, to be amplified.

Within the scope of the present invention, an amplification reaction conventionally comprises 10 to 50 or more amplification cycles, preferably about 25 to 45 cycles, particularly preferably about 40 cycles. Within the scope of the present invention, a cyclic amplification reaction preferably is a polymerase chain reaction (PCR).

A short DNA or RNA oligonucleotide having about 12 to 30 bases, which is complementary to a segment of a larger DNA or RNA molecule and which has a free 3′—OH group at its 3′ end, is usually referred to as primer. Due to said free 3′—OH group, the primer can serve as substrate for arbitrary DNA or RNA polymerases, which synthesize nucleotides to the primer in 5′ to 3′ direction. Herein, the sequence of the newly synthesized nucleotides is determined by the sequence of the template hybridized with the primer, which is located beyond the free 3′—OH group of the primer. Primers of conventional length comprise between 12 and 50 nucleotides, preferably between 15 and 30 nucleotides.

A double-stranded nucleic acid molecule or a nucleic acid strand serving as template for the synthesis of complementary nucleic acid strands is usually referred to as template or template strand.

The formation of double-stranded nucleic acid molecules or duplex molecules from complementary single-stranded nucleic acid molecules is referred to as hybridization. Herein, association preferably takes place in pairs of A and T or G and C. An association can preferably be performed via non-classic base pairings like wobble base pairings, for example between inosine and G or inosine and C. Within the scope of hybridization, for example DNA-DNA duplexes, DNA-RNA duplexes, or RNA-RNA duplexes can be formed. By means of hybridization, duplexes with nucleic acid analogs can also be formed, like for example DNA-PNA duplexes, RNA-PNA duplexes, DNA-LNA duplexes, and RNA-LNA duplexes. Hybridization experiments are usually employed in order to detect sequence complementarity and thus identity between two different nucleic acid molecules.

Herein, “specific hybridization” signifies that, under the stringent hybridization conditions described herein or known to one skilled in the art in connection with in situ and in vitro hybridization techniques, the target nucleic acids bind to the probe more strongly than the non-target nucleic acids and that essentially only the target nucleic acids, but not the non-target nucleic acids, preferably bind to the probe.

Thus, in one aspect of the present invention, a microarray device is provided comprising a support element, on which probes are immobilized on predetermined areas, for specifically detecting bacterial strains of the species Pseudomonas aeruginosa. The entirety of probes deposited in predetermined regions or in array arrangements on the detection area for specifically detecting bacterial strains of the species Pseudomonas aeruginosa and the support is also referred to as “nucleic acid chip”, “chip”, “biochip”, “microarray”, “probe array”, etc. in the following.

In particular, chips like those sold by the companies Affymetrix (Santa Clara, Calif., USA) and Clondiag (Jena, Germany) can be used within the scope of the present invention. For instance, nucleic acid chips, which are implemented in microarray devices and are described in the International Patent Applications WO 01/02094 and WO 03/031063, are used in accordance with the present invention. The disclosure of said documents concerning the arrangement of the chip in a device is hereby explicitly referred to.

Devices containing nucleic acid chips, like those described in the International Patent Application WO 03/059516, are particularly preferably used within the scope of the present invention. The disclosure of said document concerning a device for performing array methods is hereby also explicitly referred to.

Thus, a reaction tube, for example described in WO 03/059516, which has a shape and/or size typical for a laboratory reaction tube and which has a support element, on which oligonucleotide probes are immobilized on predetermined regions for specifically detecting bacterial strains of the species Pseudomonas aeruginosa, arranged on one of its base areas, is, in particular, employed as device for detecting bacterial strains of the species Pseudomonas aeruginosa.

Within the scope of the present invention, laboratory reaction tubes of typical shape and size are understood to denote reaction tubes usually utilized, in particular, in biological or molecular-biological laboratories as disposable reaction tubes, containing 1.5 ml in the standard type. Such laboratory reaction tubes are shortly denoted as “tubes” and with reference to the major manufacturer, such laboratory reaction tubes are also referred to as “Eppendorf tubes” or “Eppis” (Hamburg, Germany). Thus, laboratory reaction tubes having a typical shape and size are offered by Eppendorf as standard reaction tubes or safe-lock reaction tubes. Of course, reaction tubes having a shape and size that is typical for laboratory reaction tubes, in particular for those by Eppendorf, by manufacturers like Greiner (Frickenhausen, Germany), Millipore (Eschborn, Germany), Heraeus (Hanau, Germany), and BIOplastics (Landgraaf, Netherlands), as well as by other manufacturers may also be employed within the scope of the present invention. Examples for laboratory reaction tubes having a typical shape and size are shown in FIG. 16.

Within the scope of the present invention, laboratory reaction tubes of typical shape and size do, in particular, not denote round-bottomed flasks or other flasks like Erlenmeyer flasks, glass beakers, or measuring cylinders.

A reaction tube within the scope of the present invention is distinguished from the aforementioned reaction tubes in that it has arranged on one of its base areas a support element, on which probe molecules are immobilized on predetermined regions. Despite the modification of a conventional laboratory reaction tube by means of implementing such a chip, the reaction tube has a shape and/or size typical for a laboratory reaction tube. Thus, the reaction tube has a rotationally symmetric shape, in particular a cylindrical or substantially cylindrical shape. Of the shapes typical for conventional laboratory reaction tubes and therefore conceivable for the reaction tube according to the present invention, a conical shape deviant from the cylindrical basic shape is furthermore comprised, wherein the tapering preferably proceeds in direction toward the affinity matrix. Furthermore, typical shapes are combinations of cylindrical or substantially cylindrical regions and conical regions (see, inter alia, FIGS. 1 to 4 and 21 in WO 03/059516). Due to the shape and size typical for laboratory reaction tubes, the reaction tube with the implemented chip is, in particular, compatible with conventional table centrifuges, such as by manufacturers like Eppendorf or Heraeus, i.e. the reaction tube with nucleic acid chip is suitable for centrifugation in conventional table centrifuges. Conventional maximum external diameters for standard laboratory reaction tubes and therefore also for the reaction tube with nucleic acid chip lie in a range of 0.8 cm to 2 cm, preferably 1.0 cm to 1.5 cm, and particularly preferably 1.1 cm to 1.3 cm. Further preferred external diameters are up to 0.9 cm, up to 1.2 cm, up to 1.4 cm, up to 1.6 cm and up to 1.7 cm. Normally, the height of the laboratory reaction tube is 1.5 cm to 5.0 cm, preferably 2.0 cm to 4.0 cm, particularly preferably 2.5 cm to 3.5 cm, and most preferably 2.8 cm to 3.2 cm. Further preferred heights are up to 2.6 cm, up to 2.7 cm, up to 2.9 cm, up to 3.0 cm, up to 3.1 cm, up to 3.3 cm, and up to 3.4 cm. In special embodiments, the height can also be 1.0 cm or more.

The reaction tube with nucleic acid chip can be centrifuged in conventional table centrifuges and can thus, for example, be employed in conventional table centrifuges, like a standard table centrifuge with standard rotor by Eppendorf, as well as in conventional racks and holders for reaction tubes, like for example a tube rack by Eppendorf. For introducing the sample to be analyzed and other reagents required for performing the detection reaction into the reaction tube with nucleic acid chip, conventional pipettes or syringes, like for example variable and fixed volume pipettes by Eppendorf, can be used.

The reaction tube with nucleic acid chip has a size typical for a laboratory reaction tube. Typical filling volumes are in a range of from 100 μl to 2.5 ml, but can also be larger or smaller in special embodiments. Particularly preferably, the reaction tube has a filling volume typical for a standard Eppendorf tube of up to 1.5 ml. Further preferred filling volumes are up to 0.25 ml, up to 0.4 ml, up to 0.5 ml, up to 0.7 ml, up to 1.0 ml, or up to 2.0 ml.

In a special embodiment of the device according to the present invention, a nucleic acid chip is used, wherein a glass support together with oligonucleotides immobilized thereon is directly integrated in a 1.5 ml reaction tube, as described in the International Patent Application WO 03/059516. Clondiag sells such reaction tubes with nucleic acid chips, for example as ArrayTube®.

As already mentioned above, the nucleic acid probe in the sense of the present invention can be a DNA or RNA probe, which will normally comprise between 12 and 100 nucleotides, preferably between 15 and 50, and particularly preferably between 17 and 25 nucleotides. In a probe with a length of 15 to 25 nucleotides, complementarity should preferably be given over 100% of the sequence.

In particular, selection of the nucleic acid probes is done with respect to whether a complementary sequence is present in the strain of Pseudomonas aeruginosa to be detected.

By means of a defined sequence, which is selected as, for example, described in the following, preferably at least 20% or at least 25% and particularly preferably at least 30% or at least 35% and most preferably at least 45% or at least 50% of the population of strains of Pseudomonas aeruginosa are detected. Such selected or defined probe sequences do not provide a signal characteristic for one individual strain, however. By means of a multiplicity of different species of probes defined in such a way on the chip surface, a signal pattern is provided, however, which, with a suitable number, for example about 50 or about 70 of different probe sequences, is characteristic for each strain.

However, probes detecting a selection of more than 70% of the population of strains of Pseudomonas aeruginosa are less preferred, as the discrimination of individual strains by said probes could be too low.

Probes detecting a selection of less than 20% of the population of Pseudomonas aeruginosa are also less preferred because, while having high selectivity, they yield a signal for only few strains and thus do not contribute to information for the larger part of Pseudomonas aeruginosa strains.

In particular, the oligonucleotide probes of the nucleic acid chip according to the present invention are specific for nucleic acids having a base substitution in comparison with the sequence of the reference strain of Pseudomonas aeruginosa. The sequence of the genome of PAO1 strain, which is accessible via http://www.pseudomonas.com, is taken as reference. Preferably, the oligonucleotide probes are specific for nucleic acids having a base substitution in comparison with the sequence of conserved genes of the reference strain PAO1 of Pseudomonas aeruginosa. It is further preferred that said base substitution is present in at least 20%, at least 25%, at least 30%, at least 35%, at least 40%, and particularly preferably in at least 50% of a population of Pseudomonas aeruginosa. This means that according to the present invention single nucleotide polymorphisms (SNPs) from conserved Pseudomonas aeruginosa genes, which for example have a base substitution in at least 30% and particularly preferably in at least 50% of the population, are in particular selected for the typing. In this manner, strains of Pseudomonas aeruginosa can be determined or identified with a detection accuracy of more than 99.7%.

In a further embodiment, the nucleic acid chip of the present invention comprises, in particular in addition to the probes described in the above, oligonucleotide probes specific for nucleic acids, not present in all strains of the species Pseudomonas aeruginosa, and preferably present in at least 30% or at least 50% of the population.

In a further preferred embodiment, the nucleic acid chip according to the present invention comprises oligonucleotide probes specific for nucleic acids present in pathogenicity islets in the genome of Pseudomonas aeruginosa. Pathogenicity islets are distinct DNA regions in the genome of pathogenic bacteria, which differ from the rest of the genome concerning the presence of several pathogenicity-associated genes and a number of further structural specific features. In particular, several Pseudomonas aeruginosa strains exhibit a remarkable genomic diversity, which is essentially caused by the insertion or deletion of mobile DNA units like (pro) phages, plasmids, or other elements. Such pathogenicity islets thus also provide valuable information for discriminating different strains of Pseudomonas aeruginosa.

In a further embodiment, the nucleic acid chip according to the present invention comprises, in particular in addition to the probes suitable for discriminating different Pseudomonas aeruginosa strains, oligonucleotide probes specific for nucleic acids present in disease-associated genes like exoS and exoU. Knowledge about the presence of specific disease-associated genes allows statements on the prognosis of the patient affected and thus facilitates further treatment.

In a further embodiment, the nucleic acid chip according to the present invention comprises oligonucleotide probes specific for nucleic acids contained in genes coding for flagella of Pseudomonas aeruginosa. There are two types of flagellum for Pseudomonas aeruginosa. Information on the flagellum type of the detected Pseudomonas aeruginosa strain can provide the physician with indications of vaccines to be correspondingly administered.

In using a nucleic acid chip comprising all categories of the above-described oligonucleotide probes, i.e. probes specific for SNPs; probes specific for nucleic acids not present in all Pseudomonas aeruginosa strains; probes specific for nucleic acids in pathogenicity islets; probes specific for disease-associated genes; and probes specific for flagella-coding genes, the accuracy in determining Pseudomonas aeruginosa strains increases to more than 99.9%.

In particular, the oligonucleotide probe molecules have the following sequence lengths (all nucleic acid molecules are listed in 5′-3′ direction). The oligonucleotide probe molecules of the present invention are suitable for specifically detecting bacterial strains of the species Pseudomonas aeruginosa and, in particular, for genotyping and pathotyping the species Pseudomonas aeruginosa, and are accordingly employed, in particular, in the detection method according to the present invention. Furthermore, the oligonucleotide probes listed in the following are also suitable, however, for the use in any other methods, which are known to the person skilled in the art, for detecting or labeling bacterial strains of the species Pseudomonas aeruginosa.

In the following, those oligonucleotides or nucleic acid probe molecules are listed, which or whose modifications described below are suitable for genotyping and pathotyping of Pseudomonas aeruginosa:

GCGGAAAACTTCCTGCACATGATGTT
GCGGAAAACTTCCTCCACATGATGTT
AGCTCAGCAGACTGCTGACGAGG
AGCTCAGCAGACCGCTGACGAG
AAGAGGACGGCCGCCGGGTGACGCC
AAGAGGACGGCCGCCAGGTGACGCCG
GACAAGATGCGCCTCGACGACC
GACAAGATGCGTCTCGACGACCG
AGCCGACCTACGCGCCGGGCAG
CAGCCGACCTATGCGCCGGGCAG
CCGTTCGAACGGCTCATGGAGCA
GCCGTTCGAACGACTCATGGAGCA
TGGAGCAGCAAGTGTTCCCGGC
TGGAGCAGCAACTGTTCCCGGC
GAACAAGACCGGTTCCACCAACGG
AACAAGACCGGCTCCACCAACGG
GCGACCTGGGCCTGGTGATCCT
GCGACCTGGGACTGGTGATCCT
GCCGACCAACTGAACTCCAACTCG
GTCGCTGAACGGCACCTACTTCA
CAGCCTGCGGTCATGTCCTCGG
CGCCAGTTTGAGAACGGAGTCACC
GCGCGATCTTCTCCACTTCATCGG
GCCTCCGCGATTGAACATCGTGAT
GTAGCCGGAGTCGAGCGGAATCAT
GTGAGCATGGAATCGGCAGTCGTT
CGAGGAGTTTCGGACCCGCTTTGA
AATAGGACCGGCAGAACGGGCATT
GCGCCTTCTCCTCTTTGCAGATGT
CAGTATGGTACGGACACGAAGCGC
GCATCATTGCGCGTCACATCTGGT
TCTGAACTGCGGCTATCACCTGGA
AATTGATGGCTTCTCAGGCGCAGG
AGTCATGGGACTGAATACGGCGACT
TTCTCGGTGTCGAGGGATTCTCGG
TGGTAGCTCTCGACGTACTGGCTG
CCCGTTGCTCATAACCCGTTCCTG
AGGGCATTCTCAGGTGGACTCAGG
ACCTGTGTCGCTGGAGGGTATGTT
AGCGTCCCTGACCAACCTCATCAG
CGCCAACAATTCGCCATTACAGCG
TCCAACAGGCAGGAGTACAGGGTG
CGCTGCACATACAGGTCCGTTCTC
AGCCCAGCAATTGCGTGTTTCTCCG
AGCCCAGCAACTGCGTGTTTCTCC
GCTGCTGGCGGCGGTGTGC
TGCTGCTGGCAGCGGTGTGCT
CAGAAAGCTCAGCAGACTGCTGACGAG
GAAAGCTCAGCAGACCGCTGACGAG
ACGGCCGCCGGGTGACGCC
ACGGCCGCCAGGTGACGCCG
GCCGACCTACGCGCCGGGC
AGCCGACCTATGCGCCGGGCA
GTTCGAACGGCTCATGGAGCAGCA
GTTCGAACGACTCATGGAGCAGCAAG
CAGCCCAGTCAGGACGCGCA
AGTGACGTGCGTTTCAGCAGTCCC
GTGTCACGGCCCATGTCTAGCAGC
CGAAGTCTGAGGTGTGGACCCGC
CGCTGGAGGGTATGTTCCGCAAGG
CGTACTCAGCTTCTCCACCCAGCG
CCTGGACCTCTCCAAGGTTCGCCT
GCCATTCCGACGACCAAACAAGGC

Besides, the use of oligonucleotide probes or their modifications described below, which are specific for the following nucleic acids, is also conceivable within the scope of the present invention:

GTCTCCCTGGAGCCTGCGAAAGTGGCTCGGTTGCGTAGCCGAC
ATGTTGTATTTTTCTTGCGGTATGAAGATGGGTGGTTGGGTCGGATATAG
GTACTTCTCTCTATTTTCTTTAATTGCTCTTATCTATGG
GACCTCGACCCCCGAGGGCTTCATGGCGTGTCGCGAACTCGCATGGCAAC
AGGC
TGGTCAGCCGAGTAACCGGCAGTTGTCGCCAGGTCTGGAGAATCCCGCCA
TTAGCTTGATTCGACGGAACTATAGCGACTTTGGTCCAACTCTGGCCCAG
ATGGGCAAGAGAGTGGTTGTATTGCTATGGCTGCTATTCACATCAATGTC
AGCCCACGCCATCGATAAAAAAGTCAA
CGGCTCGGACATGGCCAATTGGGTCAGCAAGCAACGCGCCGGAGGCATGC
CTGGGTTCGCCAGGGGCGGTGCC
GTTCCTGGAACGAGGGTGATGGCTGGGAATACGTGGAGGCGCCACAGCCG
ATGTTCGTACATGACAAGCGACTGCAGTACACCGTCAGGGTCGC

The following oligonucleotide probes or their modifications described below are particularly suitable for specifically detecting SNPs in conserved genes of Pseudomonas aeruginosa:

| | |
|---|---|
| oriC T-C_wt | GAAGCCCAGCAATTGCGTGTTTC |
| oriC T-C_mut_1 | GAAGCCCAGCAACTGCGTGTTTC |
| oriC T-C_wt_1 | AGCCCAGCAATTGCGTGTTTCTCCG |
| oriC T-C_mut_2 | AGCCCAGCAACTGCGTGTTTCTCC |
| oprL T-C_wt_1 | GGTGCTGCAGGGTGTTTCGCCGG |
| oprL T-C_mut_1 | GGTGCTGCAGGGCGTTTCGCCGG |
| fliC a A-T_wt_1 | CAAGATCGCCGCAGCGGTCAAC |
| fliC a A-T_mut_1 | CAAGATCGCCGCTGCGGTCAAC |
| alkB2 G-A_wt_2 | GCTGCTGGCGGCGGTGTGC |
| alkB2 G-A_mut_2 | TGCTGCTGGCAGCGGTGTGCT |
| alkB2 A-G_wt_1 | CCTCGCCCTGTTCCCACCGCTCTGG |
| alkB2 A-G_mut_1 | CTCGCCCTGTTCCCGCCGCTCTGG |
| citS A-G_wt_1 | TCGAGCAACTGGCAGAGAAATCCG |
| citS A-G_mut_1 | CGAGCAACTGGCGGAGAAATCCG |
| citS G-C_wt_1 | GCGGAAAACTTCCTGCACATGATGTT |
| citS G-C_mut_1 | GCGGAAAACTTCCTCCACATGATGTT |
| oprI T-C_wt_1 | AGCTCAGCAGACTGCTGACGAGG |
| oprI T-C_mut_1 | AGCTCAGCAGACCGCTGACGAG |
| oprI T-C_wt_2 | CAGAAAGCTCAGCAGACTGCTGACGAG |
| oprI T-C_mut_2 | GAAAGCTCAGCAGACCGCTGACGAG |
| ampC_1 G-A_wt_2 | ACGGCCGCCGGGTGACGCC |
| ampC_1 G-A_mut_2 | ACGGCCGCCAGGTGACGCCG |
| ampC_2 C-T_wt | GACAAGATGCGCCTCGACGACC |
| ampC_2 C-T_mut_1 | GACAAGATGCGTCTCGACGACCG |
| ampC_3 C-T_wt | AGCCGACCTACGCGCCGGGCAG |
| ampC_3 C-T_mut_1 | CAGCCGACCTATGCGCCGGGCAG |
| ampC_3 C-T_wt_1 | GCCGACCTACGCGCCGGGC |
| ampC_3 C-T_mut_2 | AGCCGACCTATGCGCCGGGCA |
| ampC_4 G-A_wt_2 | GTTCGAACGGCTCATGGAGCAGCA |
| ampC_4 G-A_mut_2 | GTTCGAACGACTCATGGAGCAGCAAG |
| ampC_5 G-C_wt_1 | TGGAGCAGCAAGTGTTCCCGGC |
| ampC_5 G-C_mut_1 | TGGAGCAGCAACTGTTCCCGGC |
| ampC_6 T-C_wt | GAACAAGACCGGTTCCACCAACGG |
| ampC_6 T-C_mut_1 | AACAAGACCGGCTCCACCAACGG |
| ampC_7 C-A_wt | GCGACCTGGGCCTGGTGATCCT |
| ampC_7 C-A_mut_1 | GCGACCTGGGACTGGTGATCCT |
| oprL T-C_wt_2 | GTGCTGCAGGGTGTTTCGCCG |
| oprL T-C_mut_2 | GCTGCAGGGCGTTTCGCCG |
| oprI T-C_wt_3 | GCTCAGCAGACTGCTGACGAGGCTAACG |
| oprI T-C_mut_3 | GCTCAGCAGACCGCTGACGAGGCTAAC |
| ampC_3 C-T_wt_2 | CGACCTACGCGCCGGGCAG |
| ampC_3 C-T_mut_3 | CGACCTATGCGCCGGGCAGC |
| ampC_4 G-A_wt_3 | CGTTCGAACGGCTCATGGAGCAG |
| ampC_4 G-A_mut_3 | CGTTCGAACGACTCATGGAGCAGC |
| ampC_7 C-A_wt_1 | CGACCTGGGCCTGGTGATCCT |
| ampC_7 C-A_mut_2 | GCGACCTGGGACTGGTGATCCTGG |

The following oligonucleotide probes or their modifications described below are particularly suitable for detecting DNA sequences not present in all Pseudomonas aeruginosa strains.

| | |
|---|---|
| C-47-1 | GCGCGATCTTCTCCACTTCATCGG |
| C-45 | CGAGGAGTTTCGGACCCGCTTTGA |
| C-46 | AATAGGACCGGCAGAACGGGCATT |
| C-46_1 | CGAAGTCTGAGGTGTGGACCCGC |
| C-spezifisch-1 | GCATCATTGCGCGTCACATCTGGT |
| pKL-3 | TCTGAACTGCGGCTATCACCTGGA |
| pKL-11 | AGTCATGGGACTGAATACGGCGACT |
| PAGI-1-1 | TTCTCGGTGTCGAGGGATTCTCGG |
| PAGI-1-8 | TGGTAGCTCTCGACGTACTGGCTG |
| SG17M-1 | CCCGTTGCTCATAACCCGTTCCTG |
| SG17M-4 | AGGGCATTCTCAGGTGGACTCAGG |
| C-Inselspez.-4 | GCGCCTTCTCCTCTTTGCAGATGT |
| C-Inselspez.-5 | CAGTATGGTACGGACACGAAGCGC |
| TB-C47-3 | TCCAACAGGCAGGAGTACAGGGTG |
| TB-C47-4 | CGCTGCACATACAGGTCCGTTCTC |
| fliC a A-T_wt_2 | CAAGATCGCCGCAGCGGTCAACGAC |
| fliC a A-T_mut_2 | CAAGATCGCCGCTGCGGTCAACGAC |
| PA2221 | CAGTTGTCGCCAGGTCTGGAGAATCC |
| PA3835 | CACATCAATGTCAGCCCACGCCA |
| PA0728 | CTGGAGCCTGCGAAAGTGGCTC |
| PA2185 | ACGAGGGTGATGGCTGGGAATACG |
| PA0636 | GCCAATTGGGTCAGCAAGCAACG |
| PA0722 | CGTGTCGCGAACTCGCATGGC |
| Pyov-Rez-Type_I | CCTGAATCCGACCATTCGCGAGTC |
| Pyov-Rez-Type_IIa | TCGGACTGTACTCCTACGAAGCAGC |
| Pyov-Rez-Type_IIb | CCAATCCCTATCGCTGGAACCGTACC |
| Pyov-Rez-Type_III | GCTCGGGACTCGCATTTCGTCC |
| Pyov-Rez-Fpv_B | GCGTTATTGCTCGGTCTCTCCTCG |
| C-Inselspez.-1 | GACCGCAAGCAGAAACGGCATGC |
| C-Inselspez.-6 | CCATGGTCGGAACAGGCACGATATGC |
| C-47-1_2 | CCACTCGATCATGTTGAGCATCGGCTCC |
| SG17M-8 | GGTTAGTCCCTTCTGCCCGCATCG |

The following oligonucleotide probes or their modifications described below are particularly suitable for detecting pathogenicity islets:

| | |
|---|---|
| 47D7-1_1 | GTGTCACGGCCCATGTCTAGCAGC |
| 47D7-2 | GTGAGCATGGAATCGGCAGTCGTT |
| fla-insel-1 | ACCTGTGTCGCTGGAGGGTATGTT |
| fla-islet-2_orfA | CGCTGGAGGGTATGTTCCGCAAGG |
| fla-islet-2_orfC | CGTACTCAGCTTCTCCACCCAGCG |
| fla-islet-2_orfI | CCTGGACCTCTCCAAGGTTCGCCT |
| fla-islet-2_orfJ | GCCATTCCGACGACCAAACAAGGC |
| 47D7-2_2 | AGGCCATGGGCTAGCCGGATGC |
| PAPI-2-XF1753 | CGAAGCGTAGGGTCTTCGTAGCC |
| PAPI-2-acetyltrans | TGCGAGGACCAGAAACCTTGATGG |
| PA0980 | CGGTATGAAGATGGGTGGTTGGGTCG |
| LES | TGCATAGGAGTCATGCCGACAGCA |
| PKLC102-unbekannt | GCCTGCCTACTTGTTCCCAACGC |
| PKLC102-adhesin | GGCTGTATTGCCCGCCATTCTCC |
| PKLC102-stoffw | CGACAGACAGAAAGGGTTCTTGCGC |
| pKL-1 | CACCATGCAAATGCTCGATGGACTGC |
| TB-C47-3_2 | GCAGGCGTCCAAGTTGGAGCTCTCC |
| PAPI-1_pili-chap | GGAACACAACGTGGGGCGTGAC |
| PAPI-1_lum_bin_pro | CCAGTTGGCACCACCATGCTTGC |

The following nucleic acid probe molecules or their modifications described below are particularly suitable for detecting disease-associated genes like exoS and exoU:

| | |
|---|---|
| exoS-1_1 | CAGCCCAGTCAGGACGCGCA |
| exoU | CGCCAGTTTGAGAACGGAGTCACC |
| exoU_1 | AGTGACGTGCGTTTCAGCAGTCCC |

The following nucleic acid probe molecules or their modifications described below are particularly suitable for identifying the flagella type:

| | |
|---|---|
| fliC b | GCCGACCAACTGAACTCCAACTCG |
| fliC a | GTCGCTGAACGGCACCTACTTCA |

Besides the oligonucleotide probes having the sequences listed in the above, also modifications of the aforementioned oligonucleotides, which, despite modifications in sequence and/or length, show a specific hybridization with target nucleic acids and thereby ensure a specific detection of strains of Pseudomonas aeruginosa and, in particular, genotyping and pathotyping Pseudomonas aeruginosa, are an object of the present invention.

In particular, said modifications are
a) Nucleic acid molecules, which (i) match one of the above oligonucleotide sequences in at least 80%, preferably in at least 90%, and particularly preferably in at least 92%, 94%, 96% of the bases, or which (ii) differ from the above oligonucleotide sequences in one or more deletions and/or additions and allow a specific hybridization with target nucleic acids of strains of the species Pseudomonas aeruginosa.
b) Nucleic acid molecules hybridizing with a sequence, which is complementary to one of the nucleic acid molecules mentioned in a), under stringent conditions (see below).
c) Nucleic acid molecules comprising an oligonucleotide sequence according to a) or b) and, in addition to the sequences mentioned or modifications thereof, according to a) or b), having at least one further nucleotide and allowing a specific hybridization with target nucleic acids.

The degree of sequence identity of a nucleic acid probe molecule with the oligonucleotide probe molecules explicitly referred to in the above can be determined by means of conventional algorithms. Suitable to this end is, for example, the program for determining the sequence identity, which is accessible via http://www.ncbi.nlm.nih.gov/BLAST (on this site, for example, the link “Standard nucleotide-nucleotide BLAST [blastn]”).

Within the scope of the present invention, “hybridizing” can be synonymous with “complementary”. Within the scope of the present invention, such oligonucleotides are also comprised, which hybridize with the (theoretical) counterstrand of an oligonucleotide according to the present invention including the modifications according to the present invention.

Generally, the term “stringent conditions” denotes conditions, under which a nucleic acid sequence will preferentially bind to its target sequence, and to a distinctly lesser extent, or not at all, to other sequences. Stringent conditions are partially sequence-dependent and will be different under different circumstances. Longer sequences specifically hybridize at higher temperatures. In general, stringent conditions are selected in such a way that the temperature is about 5° C. below the thermal melting point (Tm) for the specific sequence at a defined ionic strength and a defined pH value. The melting temperature is the temperature (under defined ionic strength, pH value and nucleic acid concentration), at which 50% of the molecules complementary to the target sequence hybridize to the target sequence in a state of equilibrium.

It is understood that the person skilled in the art can select the concentrations of the components of the hybridization buffer in such a way that the desired stringency of the hybridization reaction is achieved. By means of applying said stringent conditions, the person skilled in the art is able to determine, whether a specific nucleic acid molecule allows a specific detection of target nucleic acids of Pseudomonas aeruginosa and can thus be reliably used within the scope of the present invention.

In a further preferred embodiment of the microarray device according to the present invention, so-called control probes are arranged on at least one array element. Such control probes, for example, serve for monitoring the completed labeling of the targets, the amplification reaction, the hybridization reactions, as well as—in particular in detection methods by means of precipitation—the staining of the precipitate.

Such control probes have, for example, a specific complementarity to either an externally added target or to a target present in sufficient concentration in all samples to be examined with the array. In this context, sufficient concentration is understood to denote a concentration of target molecules, which leads to a significant, i.e. clearly detectable, signal subsequently to the interaction with the probes. The array elements, on which such control probes are arranged, are preferably distributed over the entire area of the array, particularly preferably they are distributed uniformly. Within the scope of the present invention, a distribution over the entire area of the array is understood to denote that, starting from the center of the array surface, array elements with such control probes are located at different distances and in different directions. Preferably, a uniform distribution is understood to denote an arrangement of those array elements having such control probes in the form of a consistent grid, for example as 10×10 grid, wherein every tenth array element is such an array element containing control probes. This embodiment, for example, allows normalizing experimental fluctuations, which can occur subsequently to production of the array, inter alia, depending on the location of the array element on the surface of the array.

In another aspect of the present invention, a method is provided for specifically detecting bacterial strains of the species Pseudomonas aeruginosa in a sample, comprising the following steps:
a) Contacting the sample with a nucleic acid chip according to the present invention, as described in the above, having oligonucleotide probes for specifically detecting bacterial strains of the species Pseudomonas aeruginosa; and
b) Detecting the interaction between the oligonucleotide probes and the target nucleic acids contained in the sample.

The target nucleic acids to be examined or the Pseudomonas aeruginosa strains to be detected and typed can be present in any type of sample, preferably in a biological sample. In particular, the method according to the present invention will be used for examining medical samples, for example stool samples, blood cultures, sputum, tissue samples (also slices), wound material, urine, samples from the respiratory tract, implants, and catheter surfaces.

In a preferred embodiment of the detection method according to the present invention, the target nucleic acids contained in the sample are amplified before the detection. Amplification is usually performed by means of conventional PCR methods known in the art. Preferably, amplification is performed as multiplex PCR (see also WO 97/45559). In a multiplex PCR, more than one primer per template DNA is employed in the polymerase chain reaction. It is the aim of a multiplex PCR to simultaneously amplify several regions of the target DNA, thus saving time and minimizing costs.

Preferably, in an amplification by means of multiplex PCR, primers having about the same melting temperature and about the same binding kinetics are employed. In this manner, a regular amplification of all target nucleic acids and thus an exact detection of target nucleic acids, even if they are present in different initial concentrations, is ensured. Within the scope of the present invention, about the same melting temperature or a similar melting point is understood to denote a melting temperature or melting point, which preferably deviates at most 5° C. and particularly preferably at most 3° C. from the reference melting point.

In a particularly preferred embodiment, the amplification is performed linearly, i.e. only on one DNA strand of the target or template nucleic acid. It is thus avoided that even small differences in the melting points and binding kinetics of the primers, as in exponential amplification by means of conventional PCR, lead to great differences in the concentration ratios of the target nucleic acids existing after completion of the amplification, which would prevent a detection of target nucleic acids present in only low initial concentration alongside target nucleic acids present in high initial concentrations.

In particular, the primers employed within the scope of the methods of the present invention have the amounts and sequences (all primers are listed in 5′ to 3′ direction) given in the following. However, the primers listed in the following are also suitable for any other methods known to the person skilled in the art for amplifying nucleic acids.

47-1/23
ACGCGGATGTCCTGGATTTGG
47-1/39CTGAAGAAGGGGCGCTACGCG47-2/22GCGTACCGGGCAAGG
TGATAG47-2/52CTCGGTGAAACATCGGGAGGG
C45/18TCATCCAGCAAGCCATTGCGCC45/60aGGAGTCGCTTTCCGCC
ATCGC45/60bTGGAGTCGCTTTCCGCCATCGC46/15AAGGGCGTTTCA
CGCTGACGC
C46/22
ATCCGGAAGGGCGTTTCACG
C46/88
TCCACACCTCAGACTTCGGCG
C47-1/43
TATTGACGACCTACCGCGCGC
C47-2/56a
GCAACTGATGTTCGCCCAGC
C47-2/56b
CGCAACTGATGTTCGCCCAGC
C47-2/59
ACACGCAACTGATGTTCGCCC
CIS-4/36
TGTCCCGGCTCAGTTCAACG
CIS-4/50
AACACCTTGGCGTTTGTCCC
CIS-4/51
GCAACACCTTGGCGTTTGTCC
CIS-5/4
TCAAGCTCGTTGTGGACCGC
CIS-5/48
GTTACGACGGCGTGCTGTCGG
CSP-1/39a
ACGCAACGTATTCGGCGACCC
CSP-1/39b
CGCAACGTATTCGGCGACCC
fliAT/28
AGCTGATGGTATCGCCGTCGC
fliAT/72
CTAGTGATCGCACCGGAGCC
oriC/20
AGCCTCGACACCGGTTCTCG
oriC/54
TCGTTCATCCCCAGGCTTCG
oriC/59
ACCATCTCGTTCATCCCCAGG
oprL/53
TTCTGAGCCCAGGACTGCTCG
oprL/65
TCGACGCGACGGTTCTGAGCC
fliCb/36
TGACGTTCTCGCCGGTAGCG
fliCb/65
CAGTAGCGGTACCGGTCTGCG
fliCb/66
CAGTAGCGGTACCGGTCTGC
alkAG/27
TTCCTCGCCGGCATAGTAGGC
alkGA/32
GGGGTCGAGACGTGTACATGG
alkGA/51
CGAGGACGAGGCATCTTCCGG
citAG/4
GCAGGTAGCAGGTTTCCAGG
citAG/46
AACTGTTCCTTCTGCGCGGCG
citGC/8
TGATCGGCTTGGTCTCGCAGG
citGC/11
GCTGATCGGCTTGGTCTCGC
citGC/75
GAGGCGTTCTGCTCGTGGTCG
oprI/12
TTTTTCCAGCATGCGCAGGG
oprI/17
GCTGGCTTTTTCCAGCATGCG
oprI/22
TTGCGGCTGGCTTTTTCCAGC
am7CA/1
TTGGGATAGTTGCGGTTGGC
am7CA/27
CGTAGGCGATCTTCACCCGC
am7CA/29
TGGCGTAGGCGATCTTCACCC
am3CT/21
GGCGAGATAGCCGAACAGGC
am3CT/22
GCGGCGAGATAGCCGAACAGG
am3CT/69
CACTTGCTGCTCCATGAGCC
am2CT/35
GAGGTCGAGCAGGCTGATGC
am2CT/42
TAGGTCGCGAGGTCGAGCAGG
am2CT/92
GTCCTTCTGCACCGAGTCGG
am1GA/49
CGCATCTTGTCCTGGGTCAGG
am1GA/58
TCGTCGAGGCGCATCTTGTCC
am45/1
ACGTCGAGGTGGGTCTGTTCG
am45/96
GTAGCCTTCGGCATCCAGCG
am6TC/60
TCGGCATTGGGATAGTTGCGG
GI11/15
CCTCCTGTCTCATGCCGATGC
GI11/59
GCATTCGCCACGGAAGGAAGG
GI11/71
GAAGGCATCATGGCATTCGCC
GI18/62
GTCATGGGGTTTCCCAGAGACC
fliCa/41
GATCGCGATGTCGACGGTGCC
fliCa/42
CGATCGCGATGTCGACGGTGC
fliCa/46
TGCCGATCGCGATGTCGACG
SG-1/40
GACGAATACCCAGCTGCGTGG
SG-1/43
GCAGACGAATACCCAGCTGCG
SG-4/1
CGCGACGTCGTGACGTCAGC
SG-4/67
ACTTTCGGCTCTTCGGGCTGG
TB46/21
AGGTAGAGACTCGGGGGAACC
TB46/45
TCGTTTTCGGTCATGGCCAGG
TB471/22
TTCCGCGACGAACATCCGTGG
TB471/25
CGCTTCCGCGACGAACATCCG
TB472/36
GGATCGCTTCCGATAGGGCAGC
TB472/84
AGAGGCATGGGTCTGTACCG
TB473/34
TCTGTCAATCCCCTTTGGGG
TB473/41
AGCCCCTTTCTGTCAATCCCC
TB474/36
GGCTTCCTACCGAAGGTCAGG
TB474/41
TGAGGGCTTCCTACCGAAGG
exoS/31
TTCAAGGTCATGGGCAATGCC
exoS/37
AGTCCCTTCAAGGTCATGGGC
exoU/22
GCCGACTGAGCTGTAGCTCGG
exoU/23
GGCCGACTGAGCTGTAGCTCG
exoU/42
ACCAGACTGGTCAATGGTGG
flins/2
CCCGTGTTTCCGTAGACCTTGC
pKL11/49a
AGCAGTTACCCACAGCATGG
pKL11/49b
CAGCAGTTACCCACAGCATGG
pKL3/47
CTACACTCCAACCGCTGGTCC
pKL3/50
GACCTACACTCCAACCGCTGG
pKL3/80
TTCCCTTGCTGCCGAGAAGC
pKL7/14
TAATAGGCGAGCCTGCCGTCC
47D7nw1a
TCCACGCCGAGGGACGTGCC
47D7nw1b
GCTCCACGCCGAGGGACGTGCC
C46-nw1a
CGCGGTGCTGGTTGCGCTGC
C46-nw1b
CCAATGCCCAGGGCCAGCGGA
C46-nw1c
CGCTGGCAGTTCCGCTGGCC
ExoSnw1a
CAGGGTCGCCAGCTCGCTCGCC
ExoSnw1b
AGGGTCGCCAGCTCGCTCGC
ExoUnw1a
AGTGATCTGCCGCGGCCCTGCC
ExoUnw1b
GTGATCTGCCGCGGCCCTGC
OrfA-1
GTTCCACAGGCGCTGCGGCGC
OrfA-2
GTTCCACAGGCGCTGCGGCG
OrfA-3
CAAAGCCCCTGGTCGCGCGG
OrfC-1
GCAGCTTTTCCACCGCCGGCGG
OrfI-1
AAACTGCCCCGCCCCCCATCC
OrfI-2
GGAAAAACTGCCCCGCCCCCC
OrfJ-1
ACGCTCGCAGCGCCTCACGCG
OrfJ-2
GGCCTGGCTGCGAACGCTCGC
PA2221/37_Pa-P_064
TTCCTGGGCCAGAGTTGGACC
PA2221/66_Pa-P_065
AGCTTAAGGCCGTGGCACTCG
PA3835/46_Pa-P_066
CCGGAGAATTCGCGTCCACC
PA3835/72_Pa-P_067
TGCTGACGATGAAGCCCCAGC
47-22/3_Pa-P_072
AGGAGGCCGATGACAACACCC
47-22/67_Pa-P_073
TGCCGATTCCATGCTCACGCC
pI2X1753/29_Pa-P_074
ACGACGTCACCGTCGAGACCG
pI2X1753/69_Pa-P_075
ACCGCCTTTCTGGTGAGCTGG
PA0728/42_Pa-P_076
AGCCAAGACGGTTGTTCGCGG
PA0728/88_Pa-P_077
TCAATGACGCCGAGTTGGCGC
PA2185-1/42_Pa-P_078
CTCGGACAGGTTCACGCTGG
PA2185-I/70_Pa-P_079
GCCATTCGCTGCAACACCTCC
pI2actrf/39_Pa-P_085
GCGCGCGTTCGAGAAACAGG
pI2actrf/93_Pa-P_086
CGGAGGTTGAAAAGCTGGCCC
PA0636/29_Pa-P_087
ATGCCATCGTTGAAGGCACCGC
PA0636/30_Pa-P_088
TGCCATCGTTGAAGGCACCG
PA0722/4_Pa-P_089
TCTGGCGGAATCAGGTAGGCC
PA0722/55_Pa-P_090
CTTCCGGGGAGAAACCACCG
PA0980/45_Pa-P_093
ACCTCCAGCACCGACACACC
PA0980/53_Pa-P_094
ATCCGATCCACCTCCAGCACC
FpvaI/23_Pa-P_095
CGTTCAGGTCGTAGACCGCGC
FpvaI/84_Pa-P_096
GCGATACCAACTGTCCTGCGGC
FpvaIIa/34_Pa-P_097
TGCCGAAGGTGAATGGCTTGCC
FpvaIIa/65_Pa-P_098
CCTGATGGTCCGATCCCAGC
FpvaIIb/44_Pa-P_099
GCCGAGGGTCAAGAACCACTGG
FpvaIIb/67_Pa-P_100
TCTTGGCCCAGTCATAGCGGC
FpvaIII/16_Pa-P_101
TAACCCCAAGGCCCATTGGAGG
FpvaIII/31_Pa-P_102
GCCACCGCCTTCGAATAACCCC
FpvB/57_Pa-P_103
AATTGCTCGAGGGATGCGGC
FpvB/92_Pa-P_104
GGTCGAAACGGATGCGCAGG
LES/11_Pa-P_105
GCCCCGCGTCATTTTCACGTCG
LES/57_Pa-P_106
AATGCTCTGGGCAACGAGCC
pKLunbek/63_Pa-P_107
CTACCCAGCTTGGGCGTAGC
pKLunbek/141_Pa-P_108
AAGCGATAGCCGTGCTCCTGC
pKLadh/13_Pa-P_109
CCGGCTATATCCGCGGCTACC
pKLadh/59_Pa-P_110
ATTGGCGCTGCTGTTTACGCCC
pKLstw/30_Pa-P_111
GGTGGCGTCGGGTTTTTCTGC
pKLstw/46_Pa-P_112
AGGTCGTAGCGGAAGGTGGTGG
pKL-1/22_Pa-P_113
ATCTGAACCGAGGGGATCCGC
pKL-1/61_Pa-P_114
CCCGGGAGTCATTGGTCTGG
T47-32/19_Pa-P_117
GCCTGTTGGACCCCTTTGACC
T47-32/26_Pa-P_118
TACTCCTGCCTGTTGGACCCC
pI1pil/15_Pa-P_121
CGCTCAAGCGCTATCCCACC
pI1pil/41_Pa-P_122
CGCCATCGGCCTGTACAACG
pI1lumin/87_Pa-P_123
CGGTAGAGAGCTGGGTTGGC
pI1lumin/209_Pa-P_124
AACCTGGAGCTAGGGCAGAGC
C-Ins1/39_Pa-P_125
GGTGCTCGACCCAAGCATCG
C-Ins1/57_Pa-P_126
TCCTTGAGTTCCTTGGCGCGG
C-Ins6/42_Pa-P_131
CAACACGCGACTGGCGATCC
C-Ins6/61_Pa-P_132
TACATCATCCGCAACGGCGGC
C47-12/2_Pa-P_137
TATTGACGACCTACCGCGCGCC
C47-12/94_Pa-P_138
CACCAAGAACCCGCTGCTCG
SG-8/14_Pa-P_141
ATCGTGGCAGGATGTCCACCG
SG-8/86_Pa-P_142
TAGGCGGGCCTTTTGAAGGTGC

Beside primers having the above-listed sequences, modifications of the above primers, which, despite the deviations in sequence and/or length, exhibit a specific hybridization with the template nucleic acids of the respective Pseudomonas aeruginosa strains and thus are also suitable for use in amplifying the target nucleic acids, are also an object of the present invention.

Among those are, in particular,
a) primers, which (i) match one of the primer sequences explicitly mentioned in the above in at least 80%, preferably in at least 90%, and particularly preferably in at least 92%, 94%, 96% of the bases, or which (ii) differ from the above primer sequences in one or more deletions and/or additions and allow specific hybridization with template or target nucleic acids of Pseudomonas aeruginosa strains.
b) primer molecules hybridizing with a sequence, which is complementary to one of the primer molecules mentioned in a), under stringent conditions (see above).
c) nucleic acid molecules comprising the sequence of a primer molecule according to a) or b) and having, in addition to the mentioned sequences or their modifications according to a) or b), at least one further nucleotide and allowing specific hybridization with nucleic acid sequences of target organisms.

In a particularly preferred embodiment, two suitable primers per target nucleic acid are employed for the amplification in a parallel manner.

In the method according to the present invention, detection is preferably performed in that the bound or hybridized target nucleic acids are equipped with at least one label, which is detected in step b).

As already mentioned in the above, the label, coupled to the targets or probes preferably is a detectable unit or a detectable unit coupled to the targets or probes via an anchor group. With respect to the possibilities of detection or labeling, the method according to the present invention is highly adaptable. Thus, the method according to the present invention is compatible with a multiplicity of physical, chemical or biochemical detection methods. It is the only prerequisite that the unit or structure to be detected is directly coupled to a probe or target, for example an oligonucleotide, or can be linked via an anchor group, which can be coupled with the oligonucleotide.

Detection of the label can be based upon fluorescence, magnetism, charge, mass, affinity, enzymatic activity, reactivity, a gold label, and the like. Thus, the label can, for example, be based upon the use of fluorophore-labeled structures or components. In connection with fluorescence detection, the label can be any dye, which can be coupled to targets or probes during or after their synthesis. Examples are Cy dyes (Amersham Pharmacia Biotech, Uppsala, Sweden), Alexa dyes, Texas Red, Fluorescein, Rhodamin (Molecular Probes, Eugene, Oreg., USA), lanthanides such as samarium, ytterbium, and europium (EG&G, Wallac, Freiburg, Germany).

Beside fluorescence markers also luminescence markers, metal markers, enzyme markers, radioactive markers, and/or polymeric markers can be used within the scope of the present invention as labeling or detection unit, which is coupled with the targets or probes.

Likewise, a nucleic acid, which can be detected by means of hybridization with a labeled reporter (sandwich hybridization), can be used as label (tag). Diverse molecular biological detection reactions like primer extension, ligation, and RCA are employed for detecting the tag.

In an alternative embodiment of the method according to the present invention, the detectable unit is coupled with the targets or probes via an anchor group. Preferably used anchor groups are biotin, digoxigenin, and the like. In a subsequent reaction, the anchor groups are converted by means of specifically binding components, for example streptavidin conjugates or antibody conjugates, which in turn are detectable or trigger/initiate a detectable reaction. With the use of anchor groups, the conversion of the anchor groups into detectable units can be performed before, during, or after the addition of the sample comprising the targets, or, optionally, before, during, or after cleavage of the selectively cleavable bond in the probes.

According to the present invention, labeling can also be performed by means of interaction of a labeled molecule with the probe molecules. For example, labeling can be performed by means of hybridization of an oligonucleotide labeled as described above with an oligonucleotide probe or an oligonucleotide target.

Further labeling methods and detection systems suitable within the scope of the present invention are described, for example, in Lottspeich and Zorbas, Bioanalytik, Spektrum Akademischer Verlag, Heidelberg, Berlin, Germany 1998, Chapter 23.3 and 23.4.

In a preferred embodiment of the method according to the present invention, detection methods are used, which in result yield an adduct having a particular solubility product, which leads to a precipitation. For labeling, in particular substrates are used, which can be converted to a hardly soluble, usually stained product. In this labeling reaction, for example, enzymes can be used, which catalyze the conversion of a substrate to a hardly soluble product. Reactions suitable for leading to a precipitation at the array elements as well as possibilities for the detection of the precipitate are, for example, described in the International Patent Application WO 00/72018 and in the International Patent Application WO 02/02810, the contents of which are hereby explicitly referred to.

In a particularly preferred embodiment of the method according to the present invention, the bound targets are equipped with a label catalyzing the reaction of a soluble substrate to form a hardly soluble precipitate on that array element, where a probe/target interaction has taken place, or acting as a seed crystal for the conversion of a soluble substrate to a hardly soluble precipitate on that array element, where a probe/target interaction has occurred.

In this manner, the use of the method according to the present invention allows the simultaneous qualitative and quantitative analysis of a multiplicity of probe/target interactions, wherein individual array elements with a size of ≦1000 μm, preferably of ≦100 μm, and particularly preferably of ≦50 μm can be implemented.

The use of enzymatic labels is known in immunocytochemistry and in immunological tests based on microtiter plates (see E. Lidell and I. Weeks, Antibody Technology, BIOS Scientific Publishers Limited, 1995). Thus, for example, enzymes catalyze the conversion of a substrate to a hardly soluble, usually stained product.

A further possibility of detecting molecular interactions on arrays is the use of metal labels. Herein, for example colloidal gold or defined gold clusters are coupled with the targets, optionally via particular mediator molecules like streptavidin. The staining resulting from gold labeling is preferably enhanced by the subsequent reaction with less noble metals, like for example silver, wherein the gold label coupled with the targets acts as crystal nucleus or catalyst, for example, for the reduction of silver ions to a silver precipitate. The targets coupled with gold labels are also referred to as gold conjugates in the following.

In this embodiment of the method according to the present invention, a relative quantification of the probe/target interaction can also be performed. The relative quantification of the concentration of the bound targets on a probe array by detecting a precipitate is performed via the concentration of the labels coupled with the targets, which catalyze the reaction of a soluble substrate to form a hardly soluble precipitate on that array element, where a probe/target interaction has occurred, or which act as crystal nucleus for such reactions. For instance, in the case of oligonucleotide probes labeled with nanogold and purified via HPLC, the ratio of bound target to gold particles is 1:1. In other embodiments of the present invention, the ratio can be a multiple or also a fraction thereof.

Thus, in this embodiment of the inventive detection method, detection is performed by means of measuring the transmission variation, reflection, or dispersion caused by the precipitate, which is generated by the catalytic effect of the label coupled with the bound targets on those array elements, where a probe/target interaction has taken place.

In the case of coupling colloidal gold or defined gold clusters with the targets, light absorption is already evoked by the presence of said metallic labels. In order to enhance light absorption, however, a non-transparent precipitate is precipitated preferably catalytically by such interactive hybrids, i.e. targets equipped with a label like, for example, colloidal gold or defined gold clusters. In the case of gold conjugates, the use of silver as precipitate has turned out to be particularly preferable.

Thus, in a further preferred embodiment of the method according to the invention, the chronological sequence of the precipitation formation on the array elements is detected in the form of signal intensities in step c). In this manner, an exact determination of the relative quantitative amount of targets bound can be ensured. Such a procedure is described in detail in the International Patent Application WO 02/02810, the content of which is hereby explicitly referred to.

In a further aspect of the present invention, kits for performing the methods described above are provided. The hybridization set-ups or chip devices contained in said kits are, for example, described in the International Patent Applications WO 03/059516, WO 01/02094, and WO 03/031063. The disclosure contents of said documents concerning microarray devices are hereby explicitly referred to.

Apart from the hybridization set-ups described therein, preferably an ArrayTube®, the kits comprise as an important component the microarray device according to the present invention or the biochip according to the present invention and, in particular, the nucleic acid probe molecules arranged on the support and specific for Pseudomonas aeruginosa strains to be detected, as described in the above. Optionally, corresponding primers, hybridization buffers, and concentrates of corresponding washing solutions are further contained.

The following Example is supposed to explain the present invention without limiting its scope:

### EXAMPLE

Within the scope of the present invention, a detection method was developed, by means of which genotyping and pathotyping Pseudomonas aeruginosa can be performed within six hours, starting from the bacteria on an agar plate. To this end, only basic laboratory methods, like for example PCR, and devices belonging to the basic equipment of a molecular-biological laboratory are required. A critical step herein is the PCR, in which more than 40 different sequences are amplified in parallel in the same reaction setup. In order to achieve this, in one embodiment of the method according to the present invention, 80 DNA primers have been optimized in such a way that they have about the same melting points and binding kinetics. Furthermore, the template nucleic acids were only amplified linearly, i.e. on one DNA strand, thus also minimizing the effects of minor kinetic differences. Said optimization allows the use of a multiplex PCR for target amplification.

With the DNA chip provided within the scope of the present invention it is thus possible to examine Pseudomonas aeruginosa quickly and easily in a routine diagnostic laboratory within one day and thus to be able to react quickly, for example, in case nosocomial propagation of said pathogen is suspected.

An experimental protocol is given in the following:
a) Preparation of the bacteria
   taking up 2 inoculating loops of the bacterial culture (20 μl bacteria from an LB agar plate) in 1.5 ml H₂O
   centrifuging (3,000×g, 6 min)
   removing supernatant
   washing pellet 4 times
   resuspending in 5 mM EDTA
   centrifuging (14,000×g, 5 min)
   removing supernatant
   resuspending pellet in 50 μl distilled H₂O

b) Polymerase chain reaction (PCR)

The bacterial DNA sequences to be examined are amplified using polymerase chain reaction (PCR).

| Polymerase: | Terminator polymerase (New England Biolabs) | | |
|---|---|---|---|
| dNTPs: | 2 mM dATP, dGTP, dCTP each | | |
| | 1.5 mM dTTP | | |
| | 0.5 mM biotin-dUTP (Roche) | | |
| Primers: | Mixture of two 21 bp oligonucleotides each per | | |
| | sequence to be detected. The primers have the same | | |
| | melting points and binding kinetics and bind on the | | |
| | same strand, about 100 bases upstream of the | | |
| | examined DNA sequence. The mixture used has a | | |
| | total concentration of oligonucleotides of 5 μmol/l. | | |
| | The sequences of the primers used are depicted | | |
| | in FIG. 17. | | |
| Reaction setup: | 10 x reaction buffer | 2.5 | μl |
| | dNTP mixture | 2.5 | μl |
| | Primers | 2.5 | μl |
| | DMSO | 1.2 | μl |
| | Bacteria suspension | 8.0 | μl |
| | Terminator polymerase | 0.5 | μl |
| | Water | 7.8 | μl |
| | | = 25 | μl |

| | | | |
|---|---|---|---|
| Reaction procedure: | Start | 96° C. | 300 s |
| | 40 cycles | 60° C. | 20 s |
| | | 72° C. | 40 s |
| | | 96° C. | 60 s |
| | End | 10° C. | |

c) Hybridization assay

The oligonucleotide probes employed and the layout of the oligonucleotide probes on the nucleic acid chip according to the present invention are shown in FIGS. 18 to 21.

The chips are washed twice for 5 minutes with 500 μl of the hybridization buffer (6×SSPE/0.1% SDS/2% w/v Blocking Reagent (Roche)) in a thermomixer (30° C., 550 rpm).

20 μl of the PCR product are denatured together with 80 μl hybridization buffer in a heating block (96° C., 5 min) and cooled down on ice.

Said probe solution is applied onto the ArrayTube® chip (Clondiag) and incubated for one hour at 60° C. and 550 rpm (Thermomixer).

The probe solution is discarded and the DNA chip is washed:
500 μl 2×SSC/0.01% Triton X-100 for 10 min at 30° C. and 550 rpm
500 μl 2×SSC for 10 min at 20° C. and 550 rpm
500 μl 0.2×SSC for 10 min at 20° C. and 550 rpm

The ArrayTube® chip is incubated with 100 μl of an horseradish streptavidin conjugate (1:100 dilution) for 15 min (30° C., 550 rpm) and subsequently washed:
500 μl 2×SSC/0.01% Triton X-100 for 10 min at 30° C. and 550 rpm
500 μl 2×SSC for 10 min at 20° C. and 550 rpm
500 μl 0.2×SSC for 10 min at 20° C. and 550 rpm

For detection, 100 μl of a tetramethylbenzidine derivative (Medac, Wedel, Germany) are applied onto the chip and the result is evaluated by means of an AT reader (Clondiag) and the program IconoClust (Clondiag). The results for various strains of Pseudomonas aeruginosa are depicted in FIGS. 1 to 15.

d) Solutions

| 10 x SSPE buffer | 1.5 M | NaCl |
|---|---|---|
| | 0.1 M | sodium phosphate |
| | 0.01M | EDTA |
| | pH | 7.4 |
| 20 x SSC buffer | 3.0 M | NaCl |
| | 0.3 M | sodium citrate |
| | pH | 7.0 |

### FIGURES

FIGS. 1 to 15 show hybridized DNA chips, which were hybridized with different P. aeruginosa strains. Processing of the strains was performed according to the protocol described in the above.

FIG. 16 shows a laboratory reaction tube of typical shape and size.

FIG. 17 shows the nucleotide sequences of the primers used in the Example.

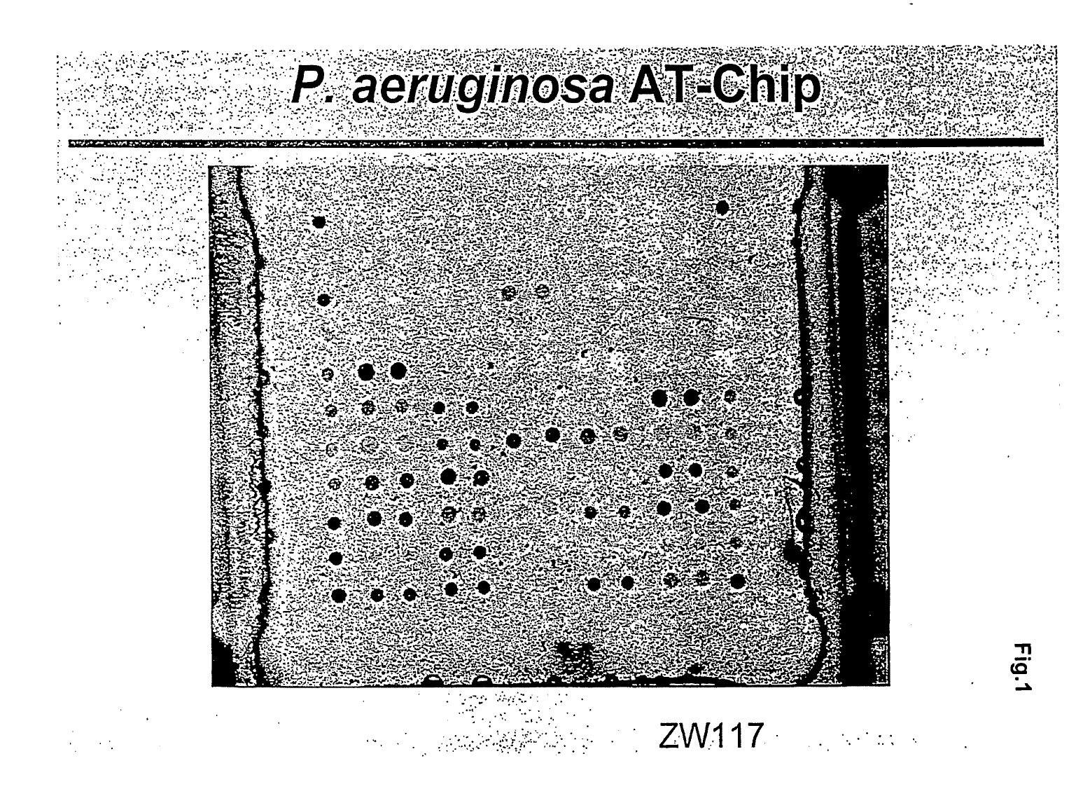

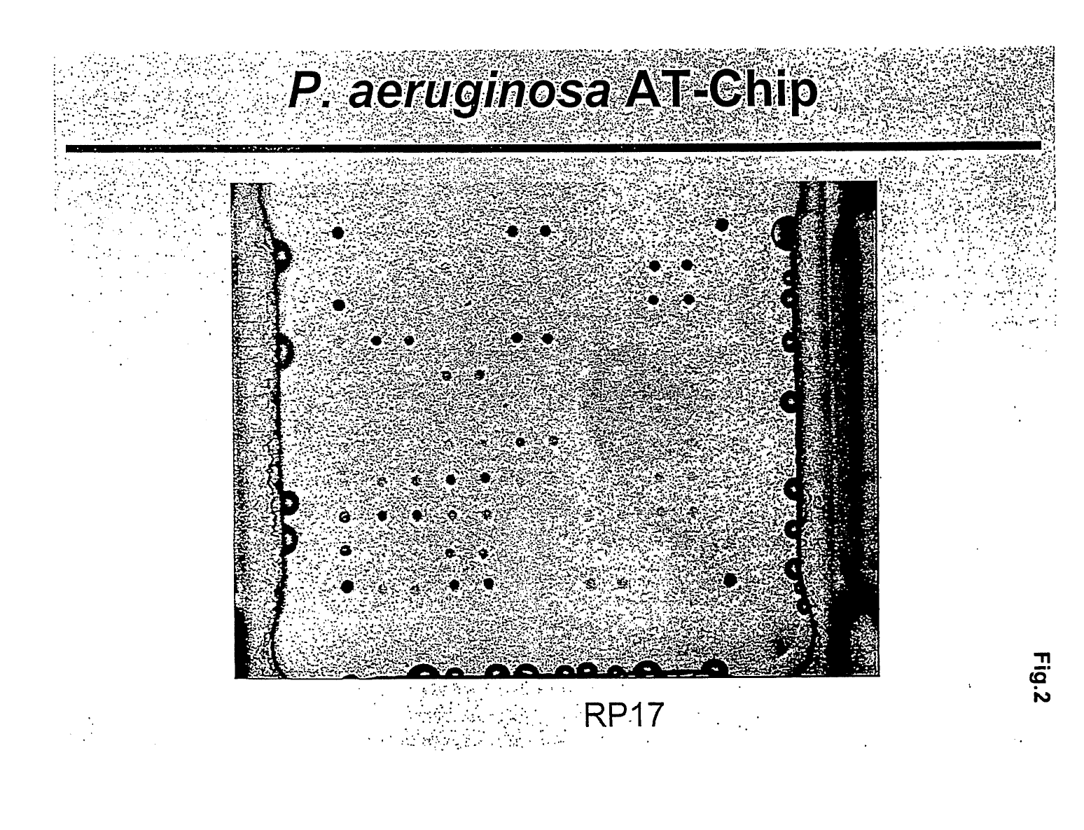

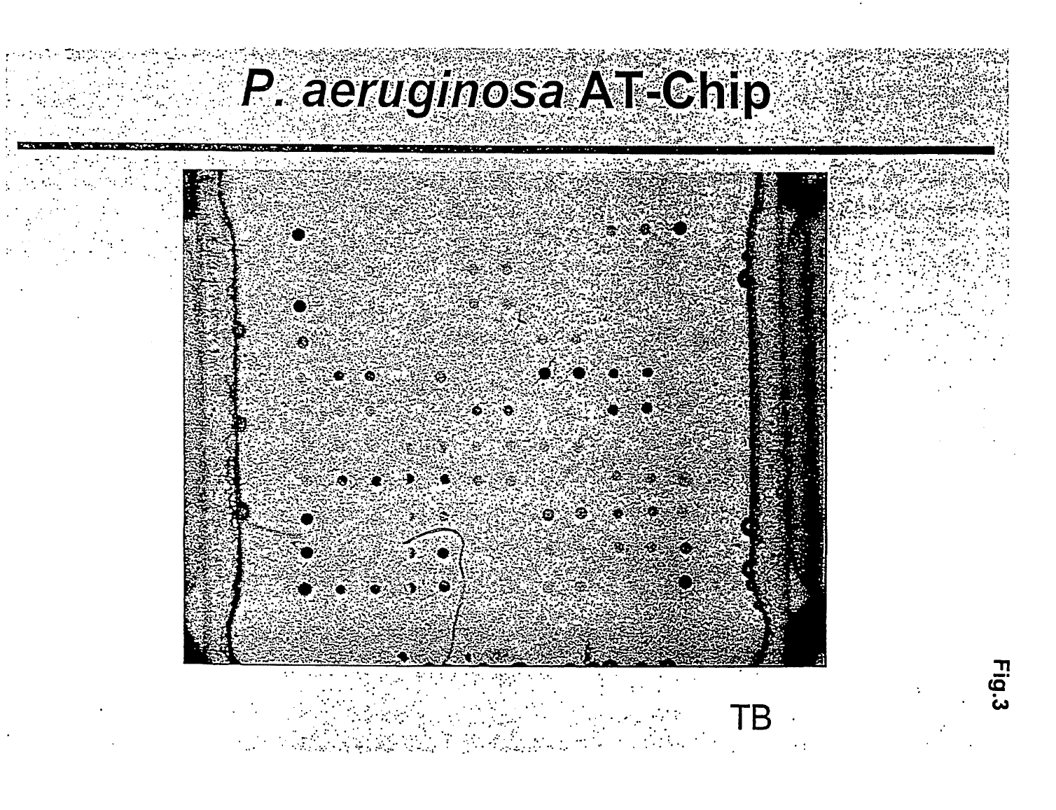

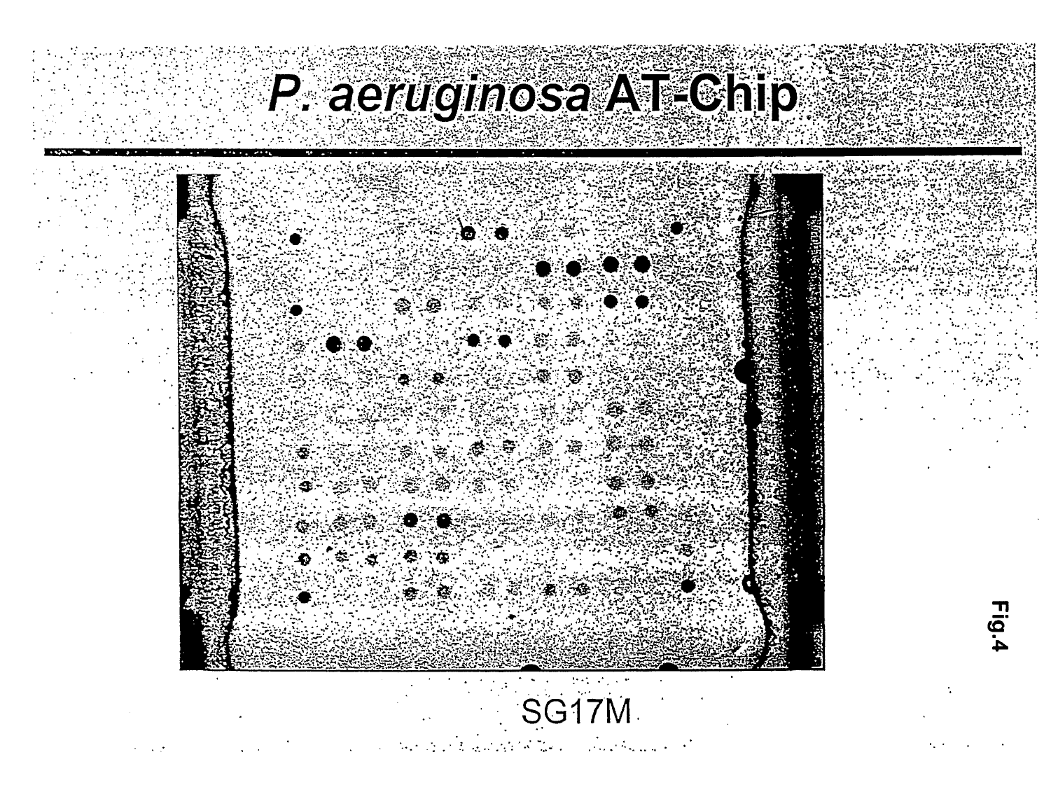

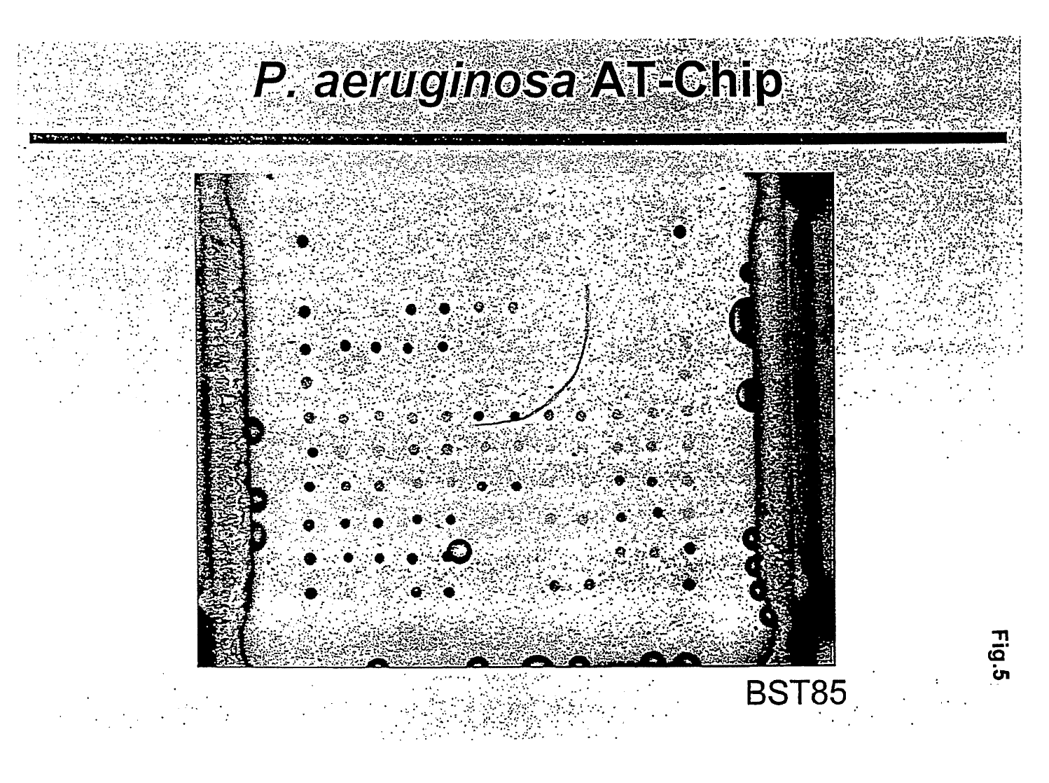

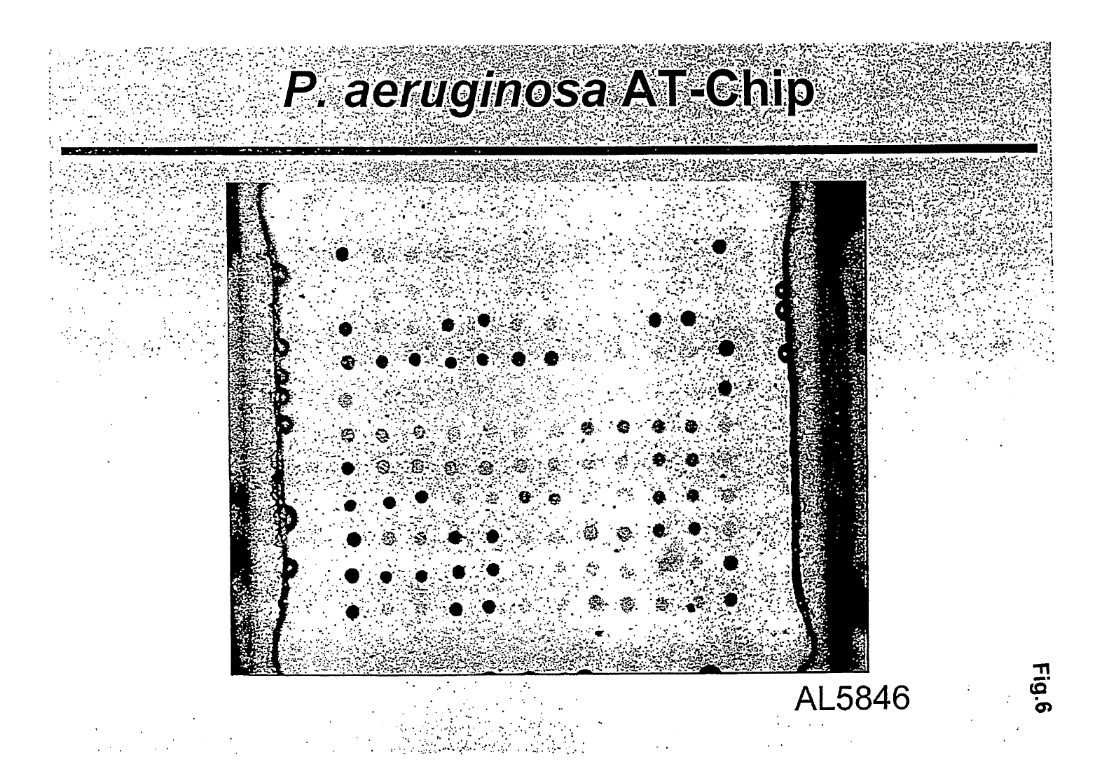

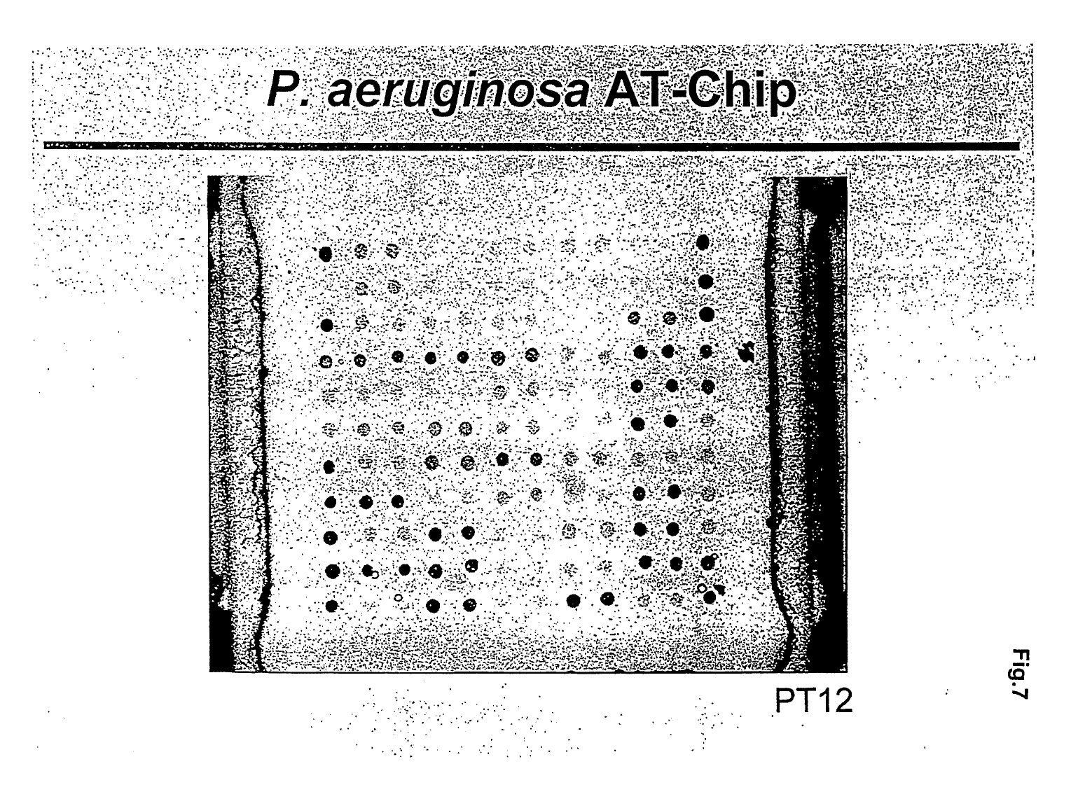

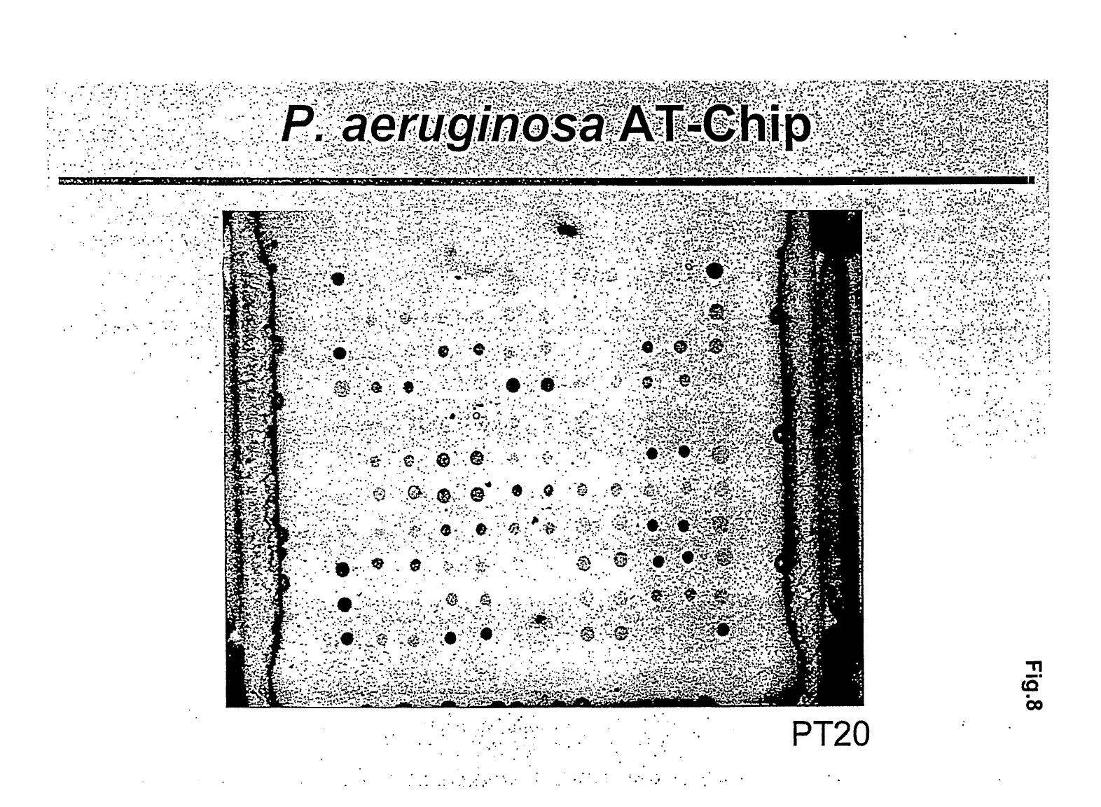

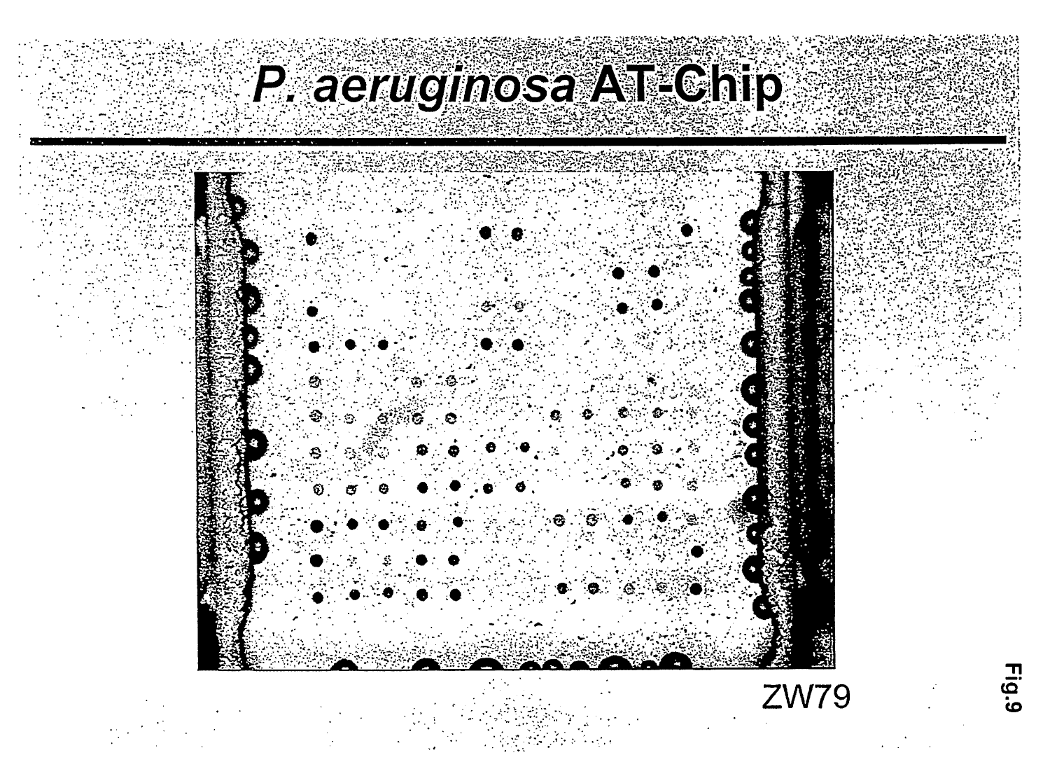

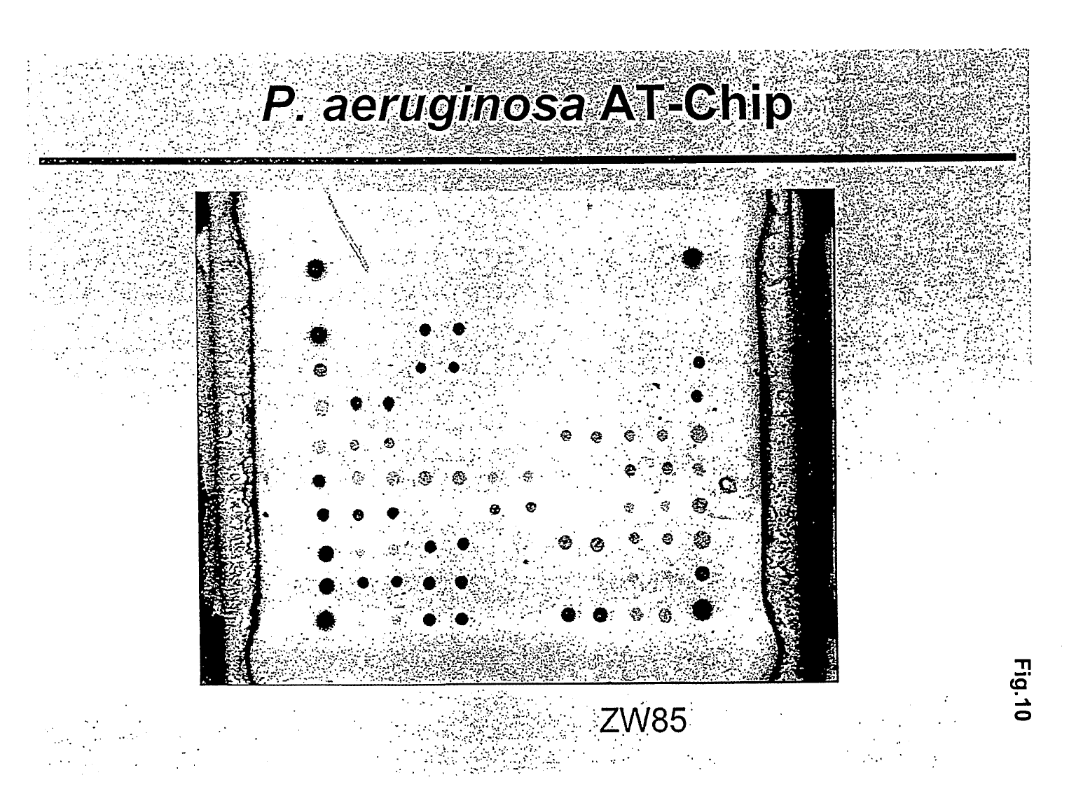

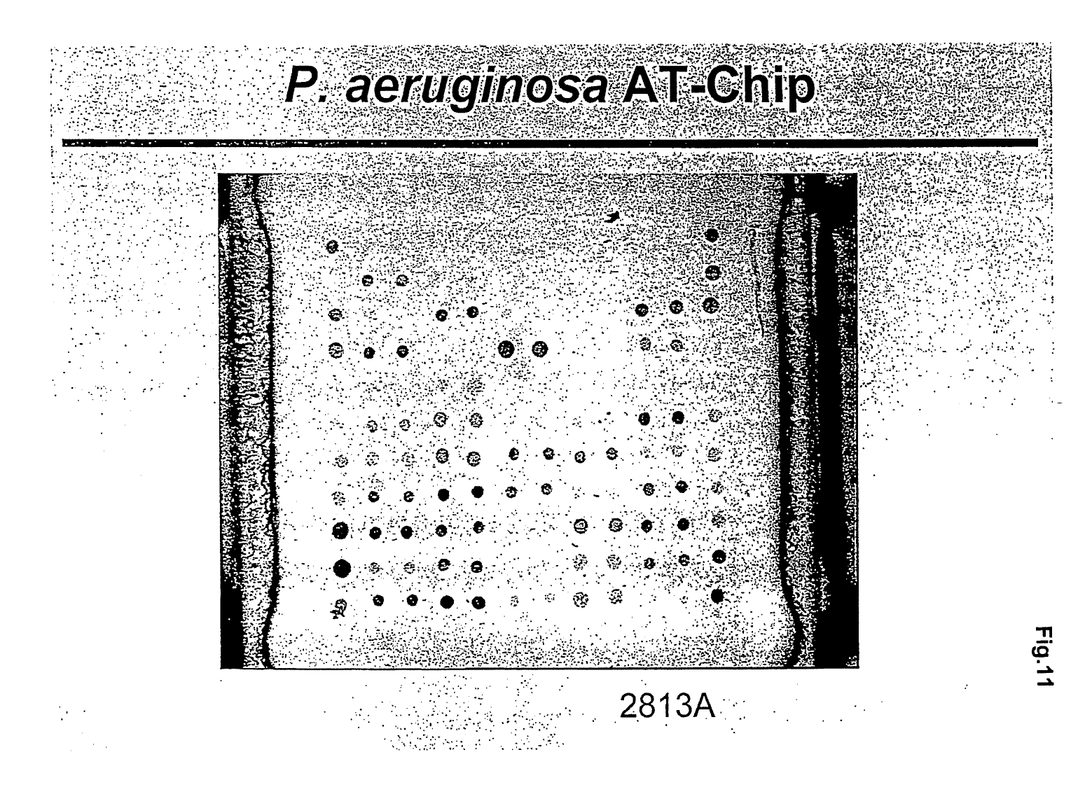

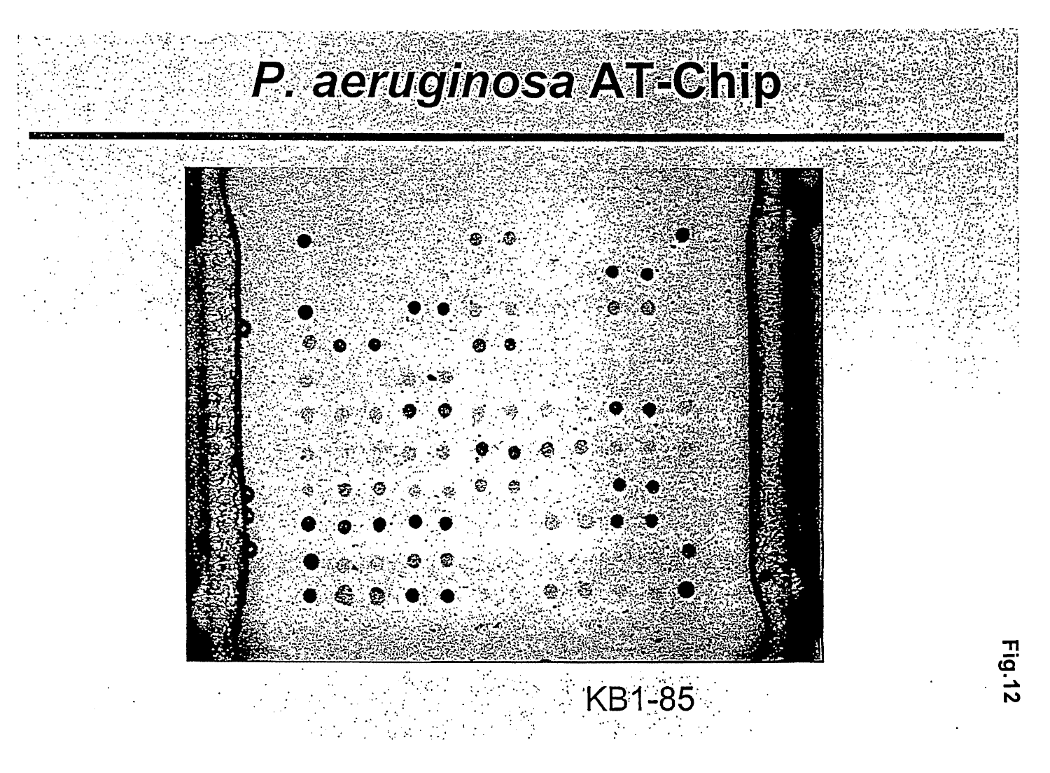

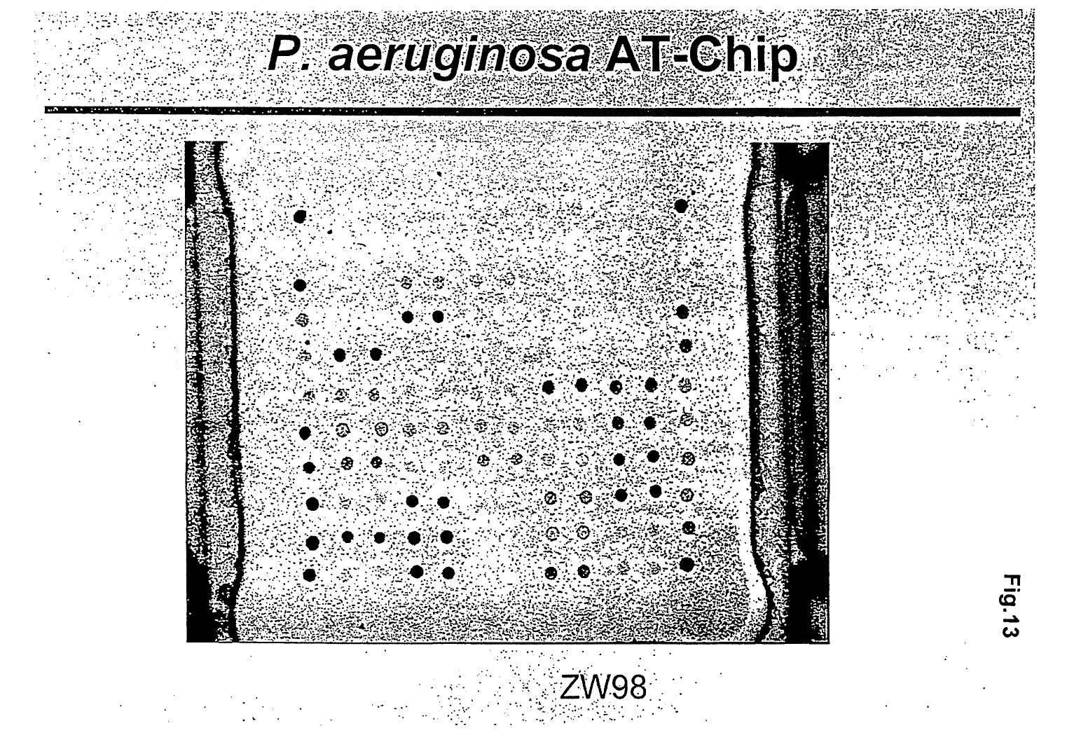

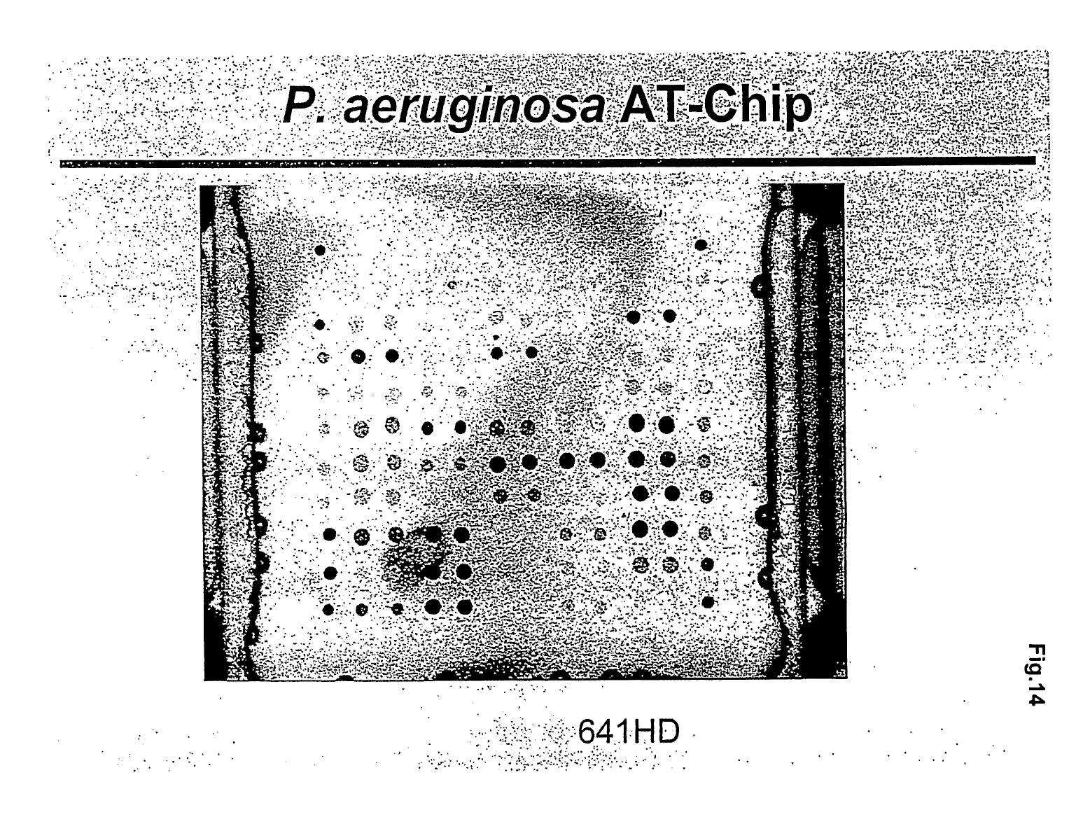

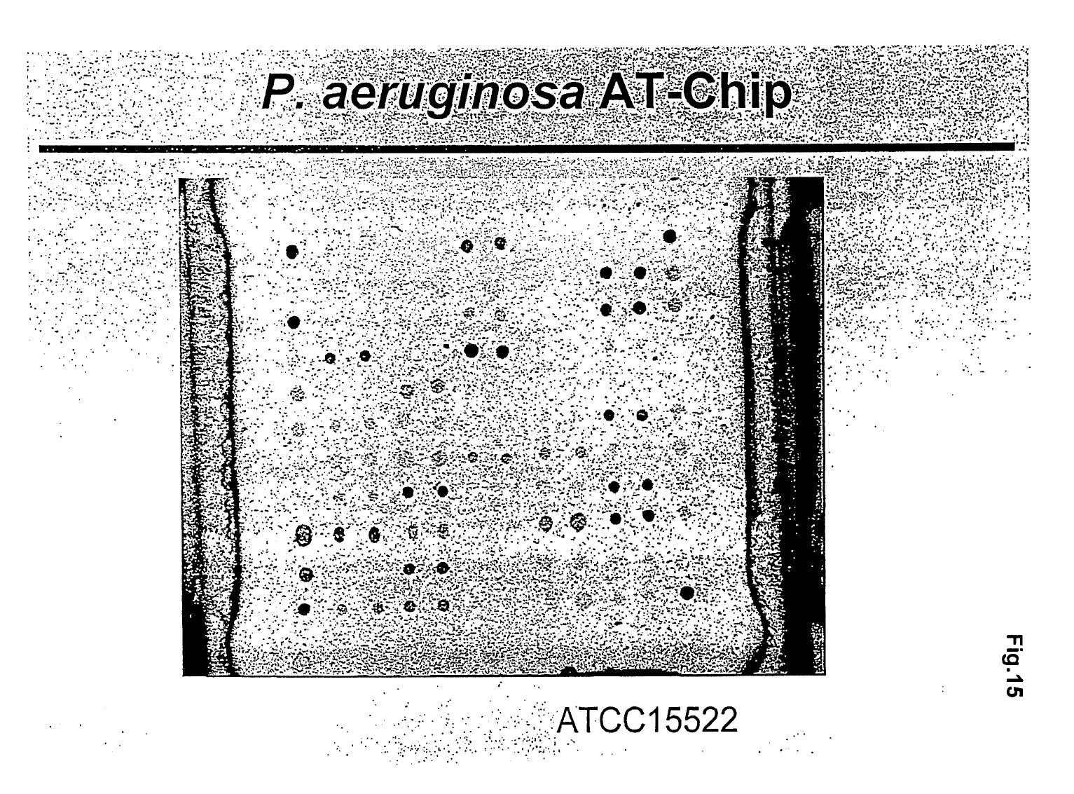

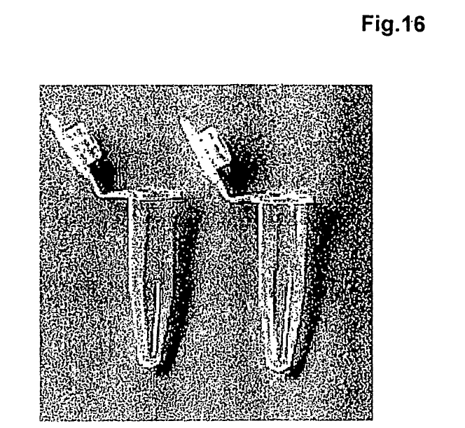

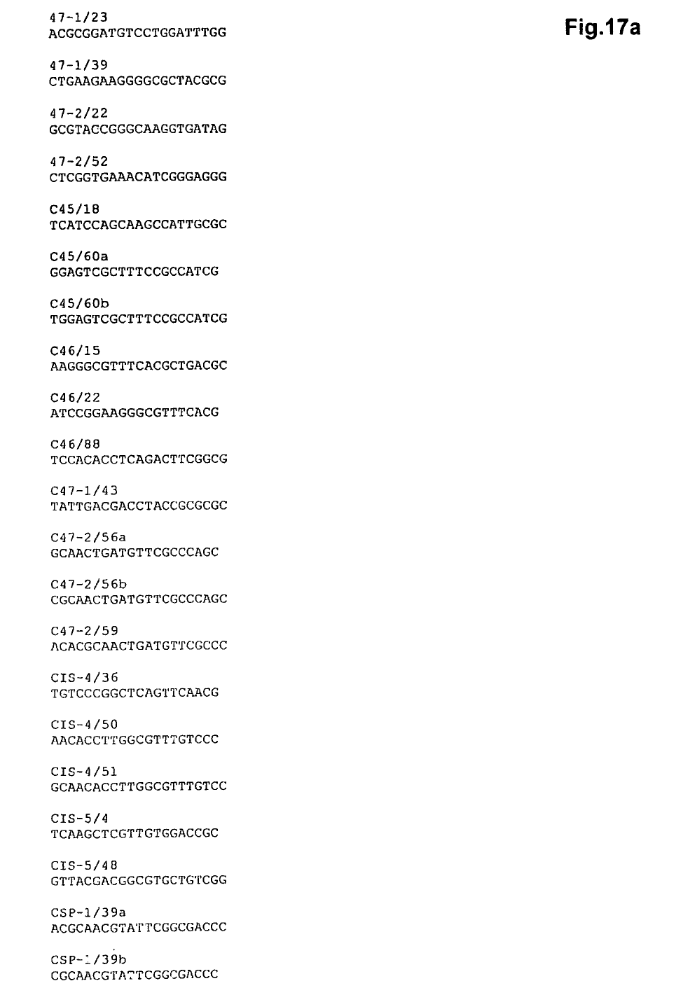

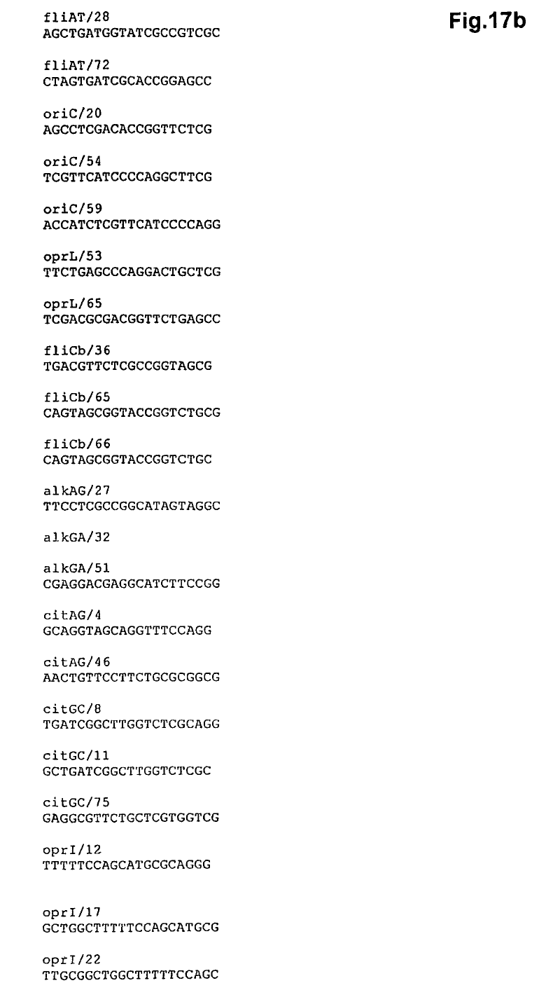

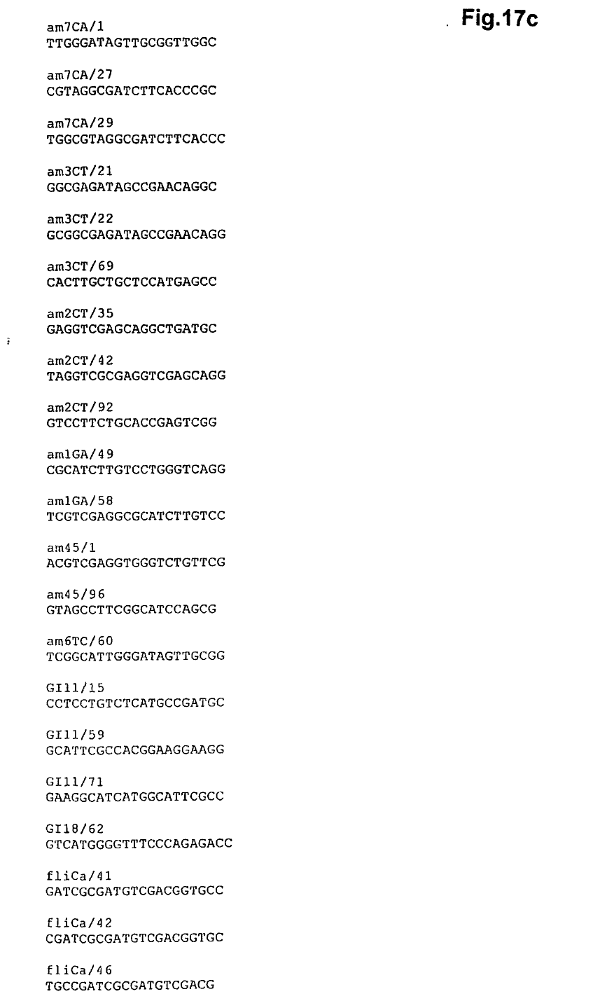

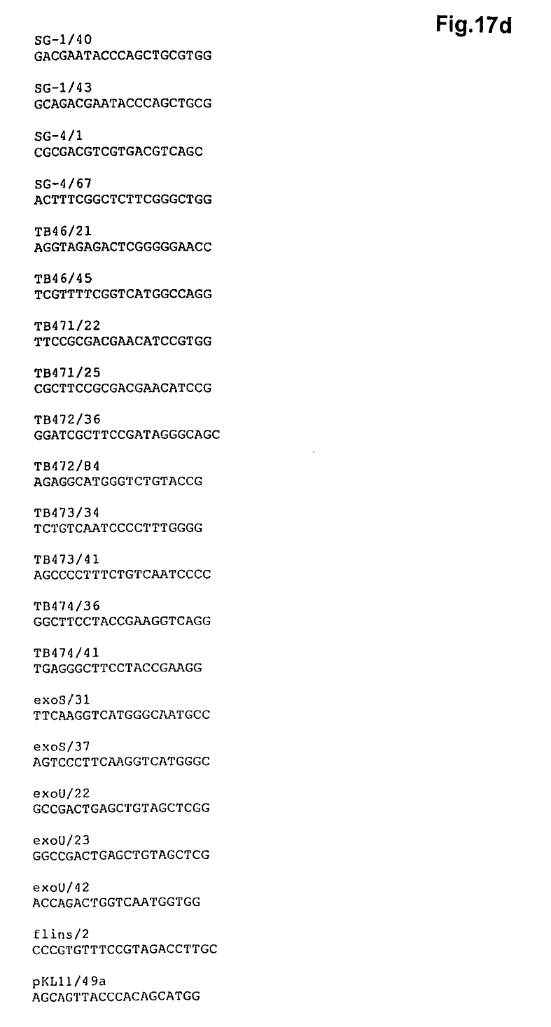

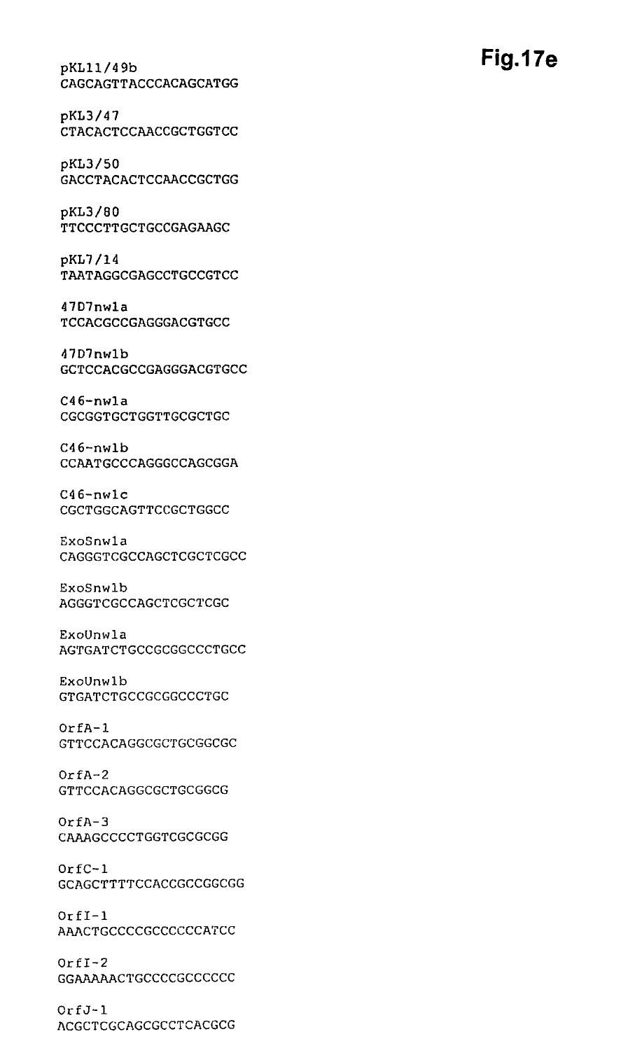

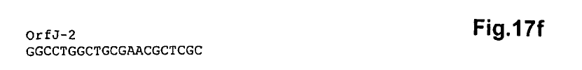

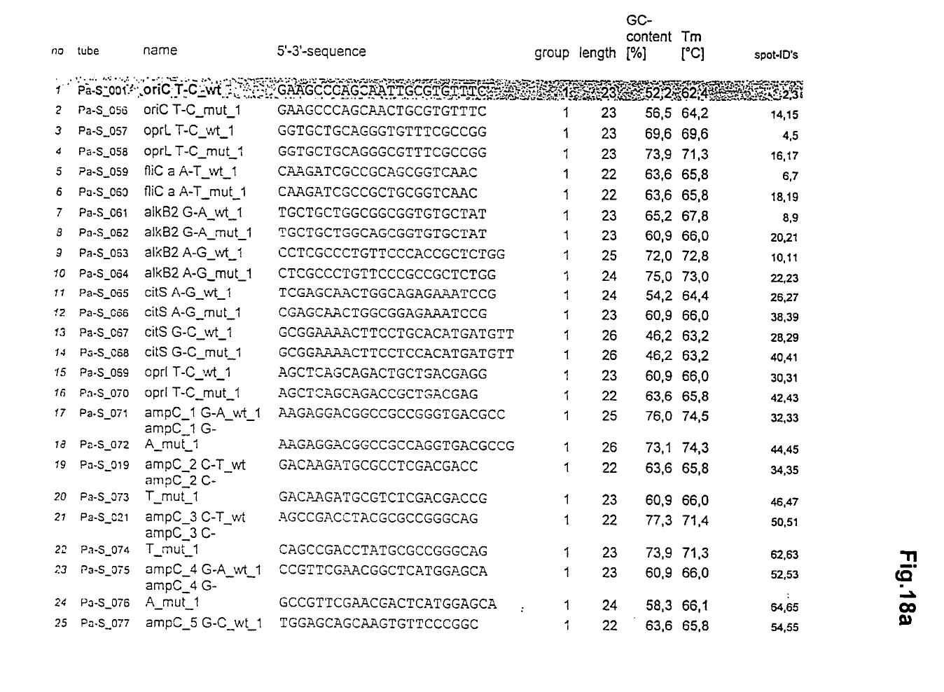

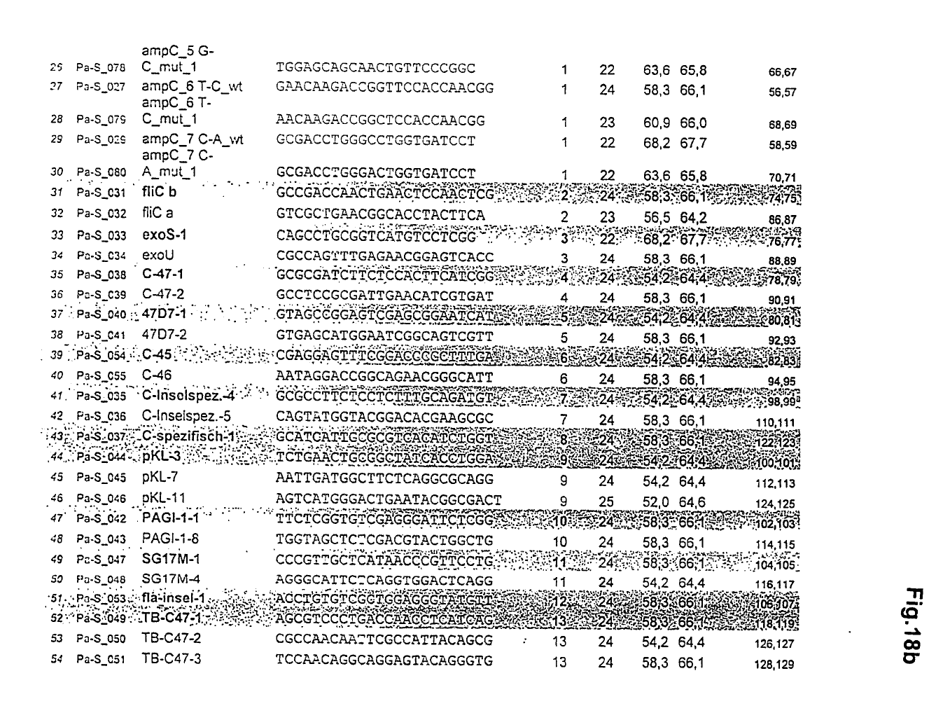

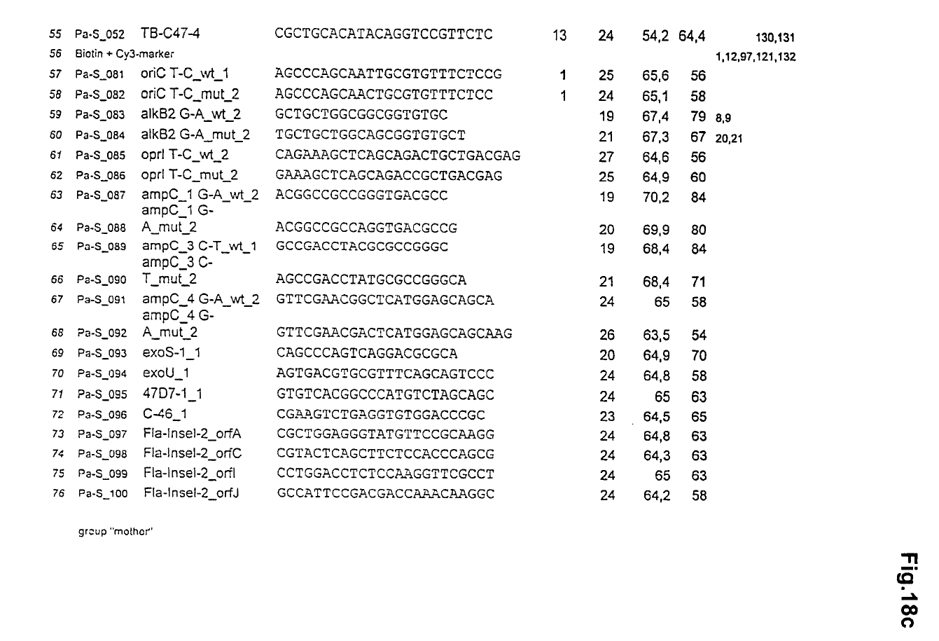

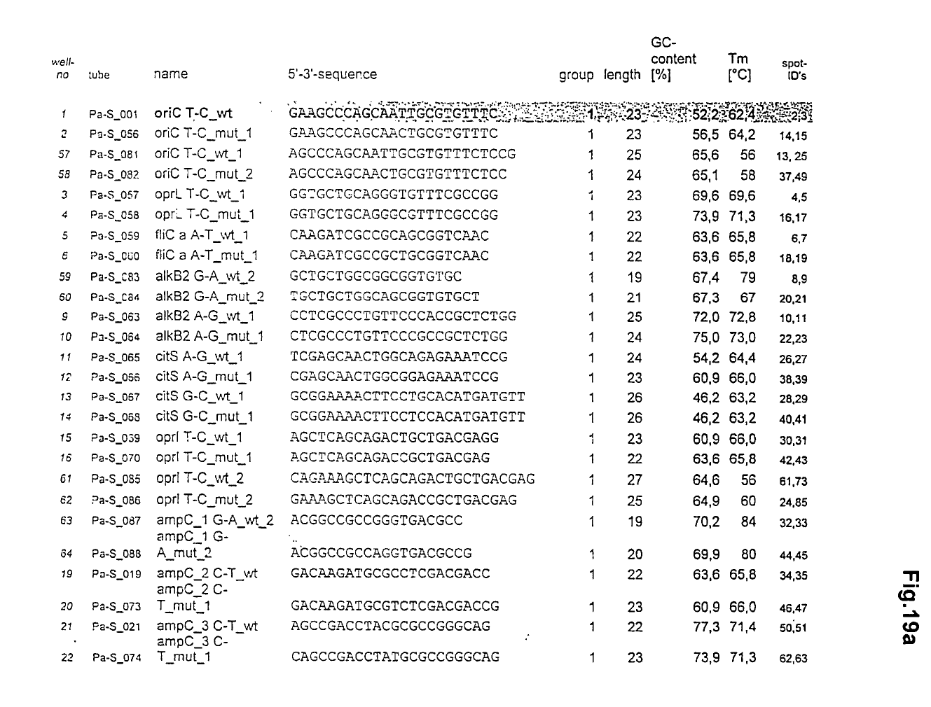

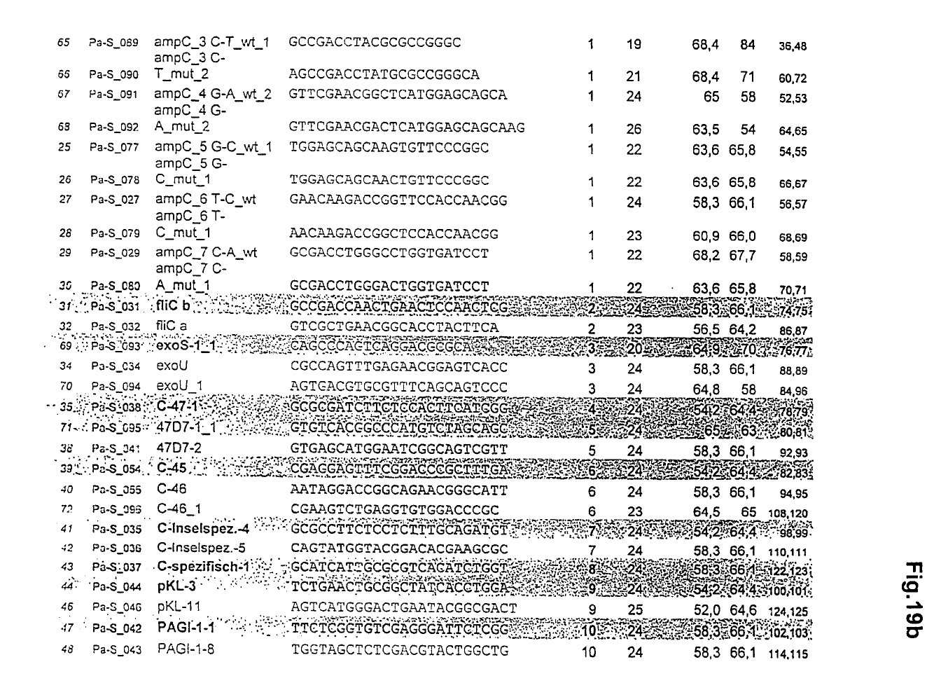

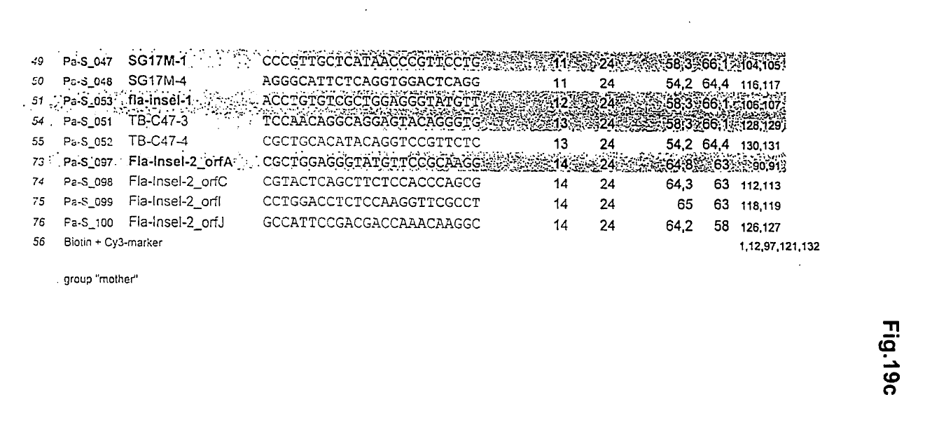

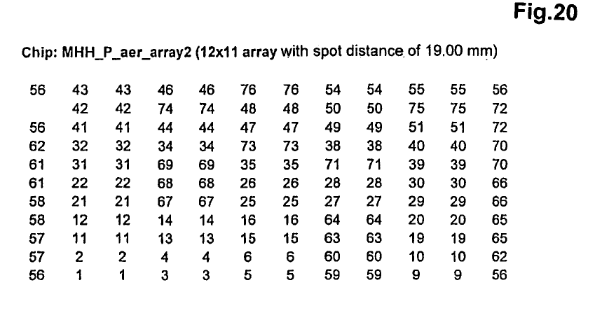

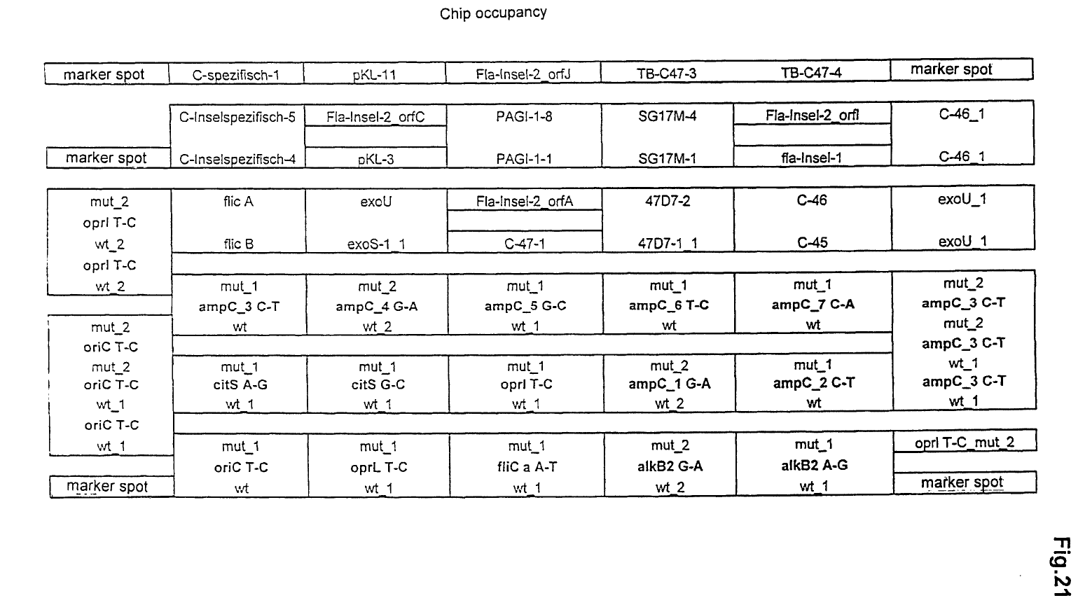

Oligonucleotide probes according to the present invention as well as the layout of the oligonucleotide probes on the nucleic acid chip according to the present invention are shown in the FIGS. 18 to 21.

1. Oligonucleotide for genotyping and pathotyping the species Pseudomonas aeruginosa with a nucleic acid sequence, selected from the group consisting of (all sequences in 5′→3′ direction):

| | |
|---|---|
| i) | |
| GAAGCCCAGCAATTGCGTGTTTC | (SEQ ID NO: 72) |
| GAAGCCCAGCAACTGCGTGTTTC | (SEQ ID NO: 73) |
| GGTGCTGCAGGGTGTTTCGCCGG | (SEQ ID NO: 76) |
| GGTGCTGCAGGGCGTTTCGCCGG | (SEQ ID NO: 77) |
| CAAGATCGCCGCAGCGGTCAAC | (SEQ ID NO: 78) |
| CAAGATCGCCGCTGCGGTCAAC | (SEQ ID NO: 79) |
| TGCTGCTGGCGGCGGTGTGCTAT | (SEQ ID NO: 80) |
| TGCTGCTGGCAGCGGTGTGCTAT | (SEQ ID NO: 81) |
| CCTCGCCCTGTTCCCACCGCTCTGG | (SEQ ID NO: 84) |
| CTCGCCCTGTTCCCGCCGCTCTGG | (SEQ ID NO: 85) |
| TCGAGCAACTGGCAGAGAAATCCG | (SEQ ID NO: 86) |
| CGAGCAACTGGCGGAGAAATCCG | (SEQ ID NO: 87) |
| GCGGAAAACTTCCTGCACATGATGTT | (SEQ ID NO: 88) |
| GCGGAAAACTTCCTCCACATGATGTT | (SEQ ID NO: 89) |
| AGCTCAGCAGACTGCTGACGAGG | (SEQ ID NO: 90) |
| AGCTCAGCAGACCGCTGACGAG | (SEQ ID NO: 91) |
| AAGAGGACGGCCGCCGGGTGACGCC | (SEQ ID NO: 5) |
| AAGAGGACGGCCGCCAGGTGACGCCG | (SEQ ID NO: 6) |
| GACAAGATGCGCCTCGACGACC | (SEQ ID NO: 7) |
| GACAAGATGCGTCTCGACGACCG | (SEQ ID NO: 8) |
| AGCCGACCTACGCGCCGGGCAG | (SEQ ID NO: 9) |
| CAGCCGACCTATGCGCCGGGCAG | (SEQ ID NO: 10) |
| CCGTTCGAACGGCTCATGGAGCA | (SEQ ID NO: 11) |
| GCCGTTCGAACGACTCATGGAGCA | (SEQ ID NO: 12) |
| TGGAGCAGCAAGTGTTCCCGGC | (SEQ ID NO: 13) |
| TGGAGCAGCAACTGTTCCCGGC | (SEQ ID NO: 14) |
| GAACAAGACCGGTTCCACCAACGG | (SEQ ID NO: 15) |
| AACAAGACCGGCTCCACCAACGG | (SEQ ID NO: 16) |
| GCGACCTGGGCCTGGTGATCCT | (SEQ ID NO: 17) |
| GCGACCTGGGACTGGTGATCC | (SEQ ID NO: 18)T |
| GCCGACCAACTGAACTCCAACTCG | (SEQ ID NO: 19) |
| GTCGCTGAACGGCACCTACTTCA | (SEQ ID NO: 20) |
| CAGCCTGCGGTCATGTCCTCGG | (SEQ ID NO: 21) |
| CGCCAGTTTGAGAACGGAGTCACC | (SEQ ID NO: 22) |
| GCGCGATCTTCTCCACTTCATCGG | (SEQ ID NO: 23) |
| GCCTCCGCGATTGAACATCGTGAT | (SEQ ID NO: 24) |
| GTAGCCGGAGTCGAGCGGAATCAT | (SEQ ID NO: 25) |
| GTGAGCATGGAATCGGCAGTCGTT | (SEQ ID NO: 26) |
| CGAGGAGTTTCGGACCCGCTTTGA | (SEQ ID NO: 27) |
| AATAGGACCGGCAGAACGGGCATT | (SEQ ID NO: 28) |
| GCGCCTTCTCCTCTTTGCAGATGT | (SEQ ID NO: 29) |
| CAGTATGGTACGGACACGAAGCGC | (SEQ ID NO: 30) |
| GCATCATTGCGCGTCACATCTGGT | (SEQ ID NO: 31) |
| TCTGAACTGCGGCTATCACCTGGA | (SEQ ID NO: 32) |
| AATTGATGGCTTCTCAGGCGCAGG | (SEQ ID NO: 33) |
| AGTCATGGGACTGAATACGGCGACT | (SEQ ID NO: 34) |
| TTCTCGGTGTCGAGGGATTCTCGG | (SEQ ID NO: 35) |
| TGGTAGCTCTCGACGTACTGGCTG | (SEQ ID NO: 36) |
| CCCGTTGCTCATAACCCGTTCCTG | (SEQ ID NO: 37) |
| AGGGCATTCTCAGGTGGACTCAGG | (SEQ ID NO: 38) |
| ACCTGTGTCGCTGGAGGGTATGTT | (SEQ ID NO: 39) |
| AGCGTCCCTGACCAACCTCATCAG | (SEQ ID NO: 40) |
| CGCCAACAATTCGCCATTACAGCG | (SEQ ID NO: 41) |
| TCCAACAGGCAGGAGTACAGGGTG | (SEQ ID NO: 42) |
| CGCTGCACATACAGGTCCGTTCTC | (SEQ ID NO: 43) |
| AGCCCAGCAATTGCGTGTTTCTCCG | (SEQ ID NO: 44) |
| AGCCCAGCAACTGCGTGTTTCTCC | (SEQ ID NO: 45) |
| GCTGCTGGCGGCGGTGTGC | (SEQ ID NO: 46) |
| TGCTGCTGGCAGCGGTGTGCT | (SEQ ID NO: 47) |
| CAGAAAGCTCAGCAGACTGCTGACGAG | (SEQ ID NO: 48) |
| GAAAGCTCAGCAGACCGCTGACGAG | (SEQ ID NO: 49) |
| ACGGCCGCCGGGTGACGCC | (SEQ ID NO: 50) |
| ACGGCCGCCAGGTGACGCCG | (SEQ ID NO: 51) |
| GCCGACCTACGCGCCGGGC | (SEQ ID NO: 52) |
| AGCCGACCTATGCGCCGGGCA | (SEQ ID NO: 53) |
| GTTCGAACGGCTCATGGAGCAGCA | (SEQ ID NO: 54) |
| GTTCGAACGACTCATGGAGCAGCAAG | (SEQ ID NO: 55) |
| CAGCCCAGTCAGGACGCGCA | (SEQ ID NO: 56) |
| AGTGACGTGCGTTTCAGCAGTCCC | (SEQ ID NO: 57) |
| GTGTCACGGCCCATGTCTAGCAGC | (SEQ ID NO: 58) |
| CGAAGTCTGAGGTGTGGACCCGC | (SEQ ID NO: 59) |
| CGCTGGAGGGTATGTTCCGCAAGG | (SEQ ID NO: 60) |
| CGTACTCAGCTTCTCCACCCAGCG | (SEQ ID NO: 61) |
| CCTGGACCTCTCCAAGGTTCGCCT | (SEQ ID NO: 62) |
| GCCATTCCGACGACCAAACAAGGC | (SEQ ID NO: 63) |
| GTGCTGCAGGGTGTTTCGCCG | (SEQ ID NO: 110) |
| GCTGCAGGGCGTTTCGCCG | (SEQ ID NO: 111) |
| CAAGATCGCCGCAGCGGTCAACGAC | (SEQ ID NO: 135) |
| CAAGATCGCCGCTGCGGTCAACGAC | (SEQ ID NO: 136) |
| GCTCAGCAGACTGCTGACGAGGCTAACG | (SEQ ID NO: 112) |
| GCTCAGCAGACCGCTGACGAGGCTAAC | (SEQ ID NO: 113) |
| CGACCTACGCGCCGGGCAG | (SEQ ID NO: 114) |
| CGACCTATGCGCCGGGCAGC | (SEQ ID NO: 115) |
| CGTTCGAACGGCTCATGGAGCAG | (SEQ ID NO: 116) |
| CGTTCGAACGACTCATGGAGCAGC | (SEQ ID NO: 117) |
| CGACCTGGGCCTGGTGATCCT | (SEQ ID NO: 118) |
| GCGACCTGGGACTGGTGATCCTGG | (SEQ ID NO: 119) |
| CAGTTGTCGCCAGGTCTGGAGAATCC | (SEQ ID NO: 137) |
| CACATCAATGTCAGCCCACGCCA | (SEQ ID NO: 138) |
| CTGGAGCCTGCGAAAGTGGCTC | (SEQ ID NO: 139) |
| ACGAGGGTGATGGCTGGGAATACG | (SEQ ID NO: 140) |
| GCCAATTGGGTCAGCAAGCAACG | (SEQ ID NO: 141) |
| CGTGTCGCGAACTCGCATGGC | (SEQ ID NO: 142) |
| AGGCCATGGGCTAGCCGGATGC | (SEQ ID NO: 159) |
| CGAAGCGTAGGGTCTTCGTAGCC | (SEQ ID NO: 160) |
| TGCGAGGACCAGAAACCTTGATGG | (SEQ ID NO: 161) |
| CGGTATGAAGATGGGTGGTTGGGTCG | (SEQ ID NO: 162) |
| CCTGAATCCGACCATTCGCGAGTC | (SEQ ID NO: 143) |
| TCGGACTGTACTCCTACGAAGCAGC | (SEQ ID NO: 144) |
| CCAATCCCTATCGCTGGAACCGTACC | (SEQ ID NO: 145) |
| GCTCGGGACTCGCATTTCGTCC | (SEQ ID NO: 146) |
| GCGTTATTGCTCGGTCTCTCCTCG | (SEQ ID NO: 147) |
| TGCATAGGAGTCATGCCGACAGCA | (SEQ ID NO: 163) |
| GCCTGCCTACTTGTTCCCAACGC | (SEQ ID NO: 164) |
| GGCTGTATTGCCCGCCATTCTCC | (SEQ ID NO: 165) |
| CGACAGACAGAAAGGGTTCTTGCGC | (SEQ ID NO: 166) |
| CACCATGCAAATGCTCGATGGACTGC | (SEQ ID NO: 167) |
| GCAGGCGTCCAAGTTGGAGCTCTCC | (SEQ ID NO: 168) |
| GGAACACAACGTGGGGCGTGAC | (SEQ ID NO: 169) |
| CCAGTTGGCACCACCATGCTTGC | (SEQ ID NO: 170) |
| GACCGCAAGCAGAAACGGCATGC | (SEQ ID NO: 148) |
| CCATGGTCGGAACAGGCACGATATGC | (SEQ ID NO: 149) |
| CCACTCGATCATGTTGAGCATCGGCTCC | (SEQ ID NO: 150) |
| GGTTAGTCCCTTCTGCCCGCATCG | (SEQ ID NO: 151) |

ii) oligonucleotides matching one of the oligonucleotides under i) in at least 60%, preferably in at least 80%, and particularly preferably in at least 90%, 92%, 94%, 96% of the bases and allowing specific hybridization with nucleic acid sequences of bacterial strains of the species Pseudomonas aeruginosa,
iii) oligonucleotides differing from one of the oligonucleotides under i) and ii) in that they are extended by at least one nucleotide, and
iv) oligonucleotides hybridizing with a sequence, which is complementary to an oligonucleotide under i), ii) and iii), under stringent conditions.

2. Microarray device comprising a support element, on which oligonucleotide probes are immobilized on predetermined regions, for specifically detecting bacterial strains of the species Pseudomonas aeruginosa.

3. Device according to claim 2, characterized in that the device is a reaction tube having a shape and/or size typical for a laboratory reaction tube and having a support element, on which oligonucleotide probes are immobilized on predetermined regions, arranged on one of its base areas for specifically detecting bacterial strains of the species Pseudomonas aeruginosa.

4. Device according to claim 2, characterized in that the oligonucleotide probes are selected in such a way that they detect 30% to 70% of the population of Pseudomonas aeruginosa strains in each case.

5. Device according to claim 2, characterized in that the oligonucleotide probes are specific for nucleic acids having a base substitution compared to the sequence of the reference strain of Pseudomonas aeruginosa.

6. Device according to claim 2, characterized in that the oligonucleotide probes are specific for nucleic acids present in only one or few strains of the species Pseudomonas aeruginosa.

7. Device according to claim 2, characterized in that the oligonucleotide probes are specific for nucleic acids present in pathogenicity islets in the genome of Pseudomonas aeruginosa.

8. Device according to claim 2, characterized in that the oligonucleotide probes are specific for nucleic acids present in disease-associated genes like exoS and exoU.

9. Device according to claim 2, characterized in that the oligonucleotide probes are specific for nucleic acids contained in genes coding for flagella of Pseudomonas aeruginosa.

10. Device according to claim 2, characterized in that the oligonucleotide probes are selected from the oligonucleotides according to claim 1.

11. Method for specifically detecting bacterial strains of the species Pseudomonas aeruginosa in a sample, comprising the following steps:
a) contacting the sample with a nucleic acid chip in a microarray device according to claim 2; and
b) detecting the interaction between the oligonucleotide probes and the target nucleic acids contained in the sample.

12. Method according to claim 11, characterized in that the target nucleic acids contained in the sample are amplified before the detection.

13. Method according to claim 12, characterized in that the amplification is performed by means of multiplex PCR.

14. Method according to claim 13, characterized in that primers, which have similar melting points and/or similar binding kinetics, are used for the amplification.

15. Method according to claim 12, characterized in that the amplification is performed linearly.

16. Method according to claim 12, characterized in that the primers are selected with a nucleic acid sequence selected from the group consisting of (all sequences in 5′→3′ direction):

| | |
|---|---|
| ACGCGGATGTCCTGGATTTGG | (SEQ ID NO: 176) |
| CTGAAGAAGGGGCGCTACGCG | (SEQ ID NO: 177) |
| GCGTACCGGGCAAGGTGATAG | (SEQ ID NO: 178) |
| CTCGGTGAAACATCGGGAGGG | (SEQ ID NO: 179) |
| TCATCCAGCAAGCCATTGCGC | (SEQ ID NO: 180) |
| GGAGTCGCTTTCCGCCATCG | (SEQ ID NO: 181) |
| TGGAGTCGCTTTCCGCCATCG | (SEQ ID NO: 182) |
| AAGGGCGTTTCACGCTGACGC | (SEQ ID NO: 183) |
| ATCCGGAAGGGCGTTTCACG | (SEQ ID NO: 184) |
| TCCACACCTCAGACTTCGGCG | (SEQ ID NO: 185) |
| TATTGACGACCTACCGCGCGC | (SEQ ID NO: 186) |
| GCAACTGATGTTCGCCCAGC | (SEQ ID NO: 187) |
| CGCAACTGATGTTCGCCCAGC | (SEQ ID NO: 188) |
| ACACGCAACTGATGTTCGCCC | (SEQ ID NO: 189) |
| TGTCCCGGCTCAGTTCAACG | (SEQ ID NO: 190) |
| AACACCTTGGCGTTTGTCCC | (SEQ ID NO: 191) |
| GCAACACCTTGGCGTTTGTCC | (SEQ ID NO: 192) |
| TCAAGCTCGTTGTGGACCGC | (SEQ ID NO: 193) |
| GTTACGACGGCGTGCTGTCGG | (SEQ ID NO: 194) |
| ACGCAACGTATTCGGCGACCC | (SEQ ID NO: 195) |
| CGCAACGTATTCGGCGACCC | (SEQ ID NO: 196) |
| AGCTGATGGTATCGCCGTCGC | (SEQ ID NO: 197) |
| CTAGTGATCGCACCGGAGCC | (SEQ ID NO: 198) |
| AGCCTCGACACCGGTTCTCG | (SEQ ID NO: 199) |
| TCGTTCATCCCCAGGCTTCG | (SEQ ID NO: 200) |
| ACCATCTCGTTCATCCCCAGG | (SEQ ID NO: 201) |
| TTCTGAGCCCAGGACTGCTCG | (SEQ ID NO: 202) |
| TCGACGCGACGGTTCTGAGCC | (SEQ ID NO: 203) |
| TGACGTTCTCGCCGGTAGCG | (SEQ ID NO: 204) |
| CAGTAGCGGTACCGGTCTGCG | (SEQ ID NO: 205) |
| CAGTAGCGGTACCGGTCTGC | (SEQ ID NO: 206) |
| TTCCTCGCCGGCATAGTAGGC | (SEQ ID NO: 207) |
| CGAGGACGAGGCATCTTCCGG | (SEQ ID NO: 209) |
| GCAGGTAGCAGGTTTCCAGG | (SEQ ID NO: 210) |
| AACTGTTCCTTCTGCGCGGCG | (SEQ ID NO: 211) |
| TGATCGGCTTGGTCTCGCAGG | (SEQ ID NO: 212) |
| GCTGATCGGCTTGGTCTCGC | (SEQ ID NO: 213) |
| GAGGCGTTCTGCTCGTGGTCG | (SEQ ID NO: 214) |
| TTTTTCCAGCATGCGCAGGG | (SEQ ID NO: 215) |
| GCTGGCTTTTTCCAGCATGCG | (SEQ ID NO: 216) |
| TTGCGGCTGGCTTTTTCCAGC | (SEQ ID NO: 217) |
| TTGGGATAGTTGCGGTTGGC | (SEQ ID NO: 218) |
| CGTAGGCGATCTTCACCCGC | (SEQ ID NO: 219) |
| TGGCGTAGGCGATCTTCACCC | (SEQ ID NO: 220) |
| GGCGAGATAGCCGAACAGGC | (SEQ ID NO: 221) |
| GCGGCGAGATAGCCGAACAGG | (SEQ ID NO: 222) |
| CACTTGCTGCTCCATGAGCC | (SEQ ID NO: 223) |
| GAGGTCGAGCAGGCTGATGC | (SEQ ID NO: 224) |
| TAGGTCGCGAGGTCGAGCAGG | (SEQ ID NO: 225) |
| GTCCTTCTGCACCGAGTCGG | (SEQ ID NO: 226) |
| CGCATCTTGTCCTGGGTCAGG | (SEQ ID NO: 227) |
| TCGTCGAGGCGCATCTTGTCC | (SEQ ID NO: 228) |
| ACGTCGAGGTGGGTCTGTTCG | (SEQ ID NO: 229) |
| GTAGCCTTCGGCATCCAGCG | (SEQ ID NO: 230) |
| TCGGCATTGGGATAGTTGCGG | (SEQ ID NO: 231) |
| CCTCCTGTCTCATGCCGATGC | (SEQ ID NO: 232) |
| GCATTCGCCACGGAAGGAAGG | (SEQ ID NO: 233) |
| GAAGGCATCATGGCATTCGCC | (SEQ ID NO: 234) |
| GTCATGGGGTTTCCCAGAGACC | (SEQ ID NO: 235) |
| GATCGCGATGTCGACGGTGCC | (SEQ ID NO: 236) |
| CGATCGCGATGTCGACGGTGC | (SEQ ID NO: 237) |
| TGCCGATCGCGATGTCGACG | (SEQ ID NO: 238) |
| GACGAATACCCAGCTGCGTGG | (SEQ ID NO: 239) |
| GCAGACGAATACCCAGCTGCG | (SEQ ID NO: 240) |
| CGCGACGTCGTGACGTCAGC | (SEQ ID NO: 241) |
| ACTTTCGGCTCTTCGGGCTGG | (SEQ ID NO: 242) |
| AGGTAGAGACTCGGGGGAACC | (SEQ ID NO: 243) |
| TCGTTTTCGGTCATGGCCAGG | (SEQ ID NO: 244) |
| TTCCGCGACGAACATCCGTGG | (SEQ ID NO: 245) |
| CGCTTCCGCGACGAACATCCG | (SEQ ID NO: 246) |
| GGATCGCTTCCGATAGGGCAGC | (SEQ ID NO: 247) |
| AGAGGCATGGGTCTGTACCG | (SEQ ID NO: 248) |
| TCTGTCAATCCCCTTTGGGG | (SEQ ID NO: 249) |
| AGCCCCTTTCTGTCAATCCCC | (SEQ ID NO: 250) |
| GGCTTCCTACCGAAGGTCAGG | (SEQ ID NO: 251) |
| TGAGGGCTTCCTACCGAAGG | (SEQ ID NO: 252) |
| TTCAAGGTCATGGGCAATGCC | (SEQ ID NO: 253) |
| AGTCCCTTCAAGGTCATGGGC | (SEQ ID NO: 254) |
| GCCGACTGAGCTGTAGCTCGG | (SEQ ID NO: 255) |
| GGCCGACTGAGCTGTAGCTCG | (SEQ ID NO: 256) |
| ACCAGACTGGTCAATGGTGG | (SEQ ID NO: 257) |
| CCCGTGTTTCCGTAGACCTTGC | (SEQ ID NO: 258) |
| AGCAGTTACCCACAGCATGG | (SEQ ID NO: 259) |
| CAGCAGTVACCCACAGCATGG | (SEQ ID NO: 260) |
| CTACACTCCAACCGCTGGTCC | (SEQ ID NO: 261) |
| GACCTACACTCCAACCGCTGG | (SEQ ID NO: 262) |
| TTCCCTTGCTGCCGAGAAGC | (SEQ ID NO: 263) |
| TAATAGGCGAGCCTGCCGTCC | (SEQ ID NO: 264) |
| TCCACGCCGAGGGACGTGCC | (SEQ ID NO: 265) |
| GCTCCACGCCGAGGGACGTGCC | (SEQ ID NO: 266) |
| CGCGGTGCTGGTTGCGCTGC | (SEQ ID NO: 267) |
| CCAATGCCCAGGGCCAGCGGA | (SEQ ID NO: 268) |
| CGCTGGCAGTTCCGCTGGCC | (SEQ ID NO: 269) |
| CAGGGTCGCCAGCTCGCTCGCC | (SEQ ID NO: 270) |
| AGGGTCGCCAGCTCGCTCGC | (SEQ ID NO: 271) |
| AGTGATCTGCCGCGGCCCTGCC | (SEQ ID NO: 272) |
| GTGATCTGCCGCGGCCCTGC | (SEQ ID NO: 273) |
| GTTCCACAGGCGCTGCGGCGC | (SEQ ID NO: 274) |
| GTTCCACAGGCGCTGCGGCG | (SEQ ID NO: 275) |
| CAAAGCCCCTGGTCGCGCGG | (SEQ ID NO: 276) |
| GCAGCTTTTCCACCGCCGGCGG | (SEQ ID NO: 277) |
| AAACTGCCCCGCCCCCCATCC | (SEQ ID NO: 278) |
| GGAAAAACTGCCCCGCCCCCC | (SEQ ID NO: 279) |
| ACGCTCGCAGCGCCTCACGCG | (SEQ ID NO: 280) |
| GGCCTGGCTGCGAACGCTCGC | (SEQ ID NO: 281) |
| GGGGTCGAGACGTGTACATGG | (SEQ ID NO: 208) |
| TTCCTGGGCCAGAGTTGGACC | (SEQ ID NO: 282) |
| AGCTTAAGGCCGTGGCACTCG | (SEQ ID NO: 283) |
| CCGGAGAATTCGCGTCCACC | (SEQ ID NO: 284) |
| TGCTGACGATGAAGCCCCAGC | (SEQ ID NO: 285) |
| AGGAGGCCGATGACAACACCC | (SEQ ID NO: 286) |
| TGCCGATTCCATGCTCACGCC | (SEQ ID NO: 287) |
| ACGACGTCACCGTCGAGACCG | (SEQ ID NO: 288) |
| ACCGCCTTTCTGGTGAGCTGG | (SEQ ID NO: 289) |
| AGCCAAGACGGTTGTTCGCGG | (SEQ ID NO: 290) |
| TCAATGACGCCGAGTTGGCGC | (SEQ ID NO: 291) |
| CTCGGACAGGTTCACGCTGG | (SEQ ID NO: 292) |
| GCCATTCGCTGCAACACCTCC | (SEQ ID NO: 293) |
| GCGCGCGTTCGAGAAACAGG | (SEQ ID NO: 294) |
| CGGAGGTTGAAAAGCTGGCCC | (SEQ ID NO: 295) |
| ATGCCATCGTTGAAGGCACCGC | (SEQ ID NO: 296) |
| TGCCATCGTTGAAGGCACCG | (SEQ ID NO: 297) |
| TCTGGCGGAATCAGGTAGGCC | (SEQ ID NO: 298) |
| CTTCCGGGGAGAAACCACCG | (SEQ ID NO: 299) |
| ACCTCCAGCACCGACACACC | (SEQ ID NO: 300) |
| ATCCGATCCACCTCCAGCACC | (SEQ ID NO: 301) |
| CGTTCAGGTCGTAGACCGCGC | (SEQ ID NO: 302) |
| GCGATACCAACTGTCCTGCGGC | (SEQ ID NO: 303) |
| TGCCGAAGGTGAATGGCTTGCC | (SEQ ID NO: 304) |
| CCTGATGGTCCGATCCCAGC | (SEQ ID NO: 305) |
| GCCGAGGGTCAAGAACCACTGG | (SEQ ID NO: 306) |
| TCTTGGCCCAGTCATAGCGGC | (SEQ ID NO: 307) |
| TAACCCCAAGGCCCATTGGAGG | (SEQ ID NO: 308) |
| GCCACCGCCTTCGAATAACCCC | (SEQ ID NO: 309) |
| AATTGCTCGAGGGATGCGGC | (SEQ ID NO: 310) |
| GGTCGAAACGGATGCGCAGG | (SEQ ID NO: 311) |
| GCCCCGCGTCATTTTCACGTCG | (SEQ ID NO: 312) |
| AATGCTCTGGGCAACGAGCC | (SEQ ID NO: 313) |
| CTACCCAGCTTGGGCGTAGC | (SEQ ID NO: 314) |
| AAGCGATAGCCGTGCTCCTGC | (SEQ ID NO: 315) |
| CCGGCTATATCCGCGGCTACC | (SEQ ID NO: 316) |
| ATTGGCGCTGCTGTTTACGCCC | (SEQ ID NO: 317) |
| GGTGGCGTCGGGTTTTTCTGC | (SEQ ID NO: 318) |
| AGGTCGTAGCGGAAGGTGGTGG | (SEQ ID NO: 319) |
| ATCTGAACCGAGGGGATCCGC | (SEQ ID NO: 320) |
| CCCGGGAGTCATTGGTCTGG | (SEQ ID NO: 321) |
| GCCTGTTGGACCCCTTTGACC | (SEQ ID NO: 322) |
| TACTCCTGCCTGTTGGACCCC | (SEQ ID NO: 323) |
| CGCTCAAGCGCTATCCCACC | (SEQ ID NO: 324) |
| CGCCATCGGCCTGTACAACG | (SEQ ID NO: 325) |
| CGGTAGAGAGCTGGGTTGGC | (SEQ ID NO: 326) |
| AACCTGGAGCTAGGGCAGAGC | (SEQ ID NO: 327) |
| GGTGCTCGACCCAAGCATCG | (SEQ ID NO: 328) |
| TCCTTGAGTTCCTTGGCGCGG | (SEQ ID NO: 329) |
| CAACACGCGACTGGCGATCC | (SEQ ID NO: 330) |
| TACATCATCCGCAACGGCGGC | (SEQ ID NO: 331) |
| TATTGACGACCTACCGCGCGCC | (SEQ ID NO: 332) |
| CACCAAGAACCCGCTGCTCG | (SEQ ID NO: 333) |
| ATCGTGGCAGGATGTCCACCG | (SEQ ID NO: 334) |
| TAGGCGGGCCTTTTGAAGGTGC | (SEQ ID NO: 335) |

17. Use of the oligonucleotides according to claim 1 for specifically detecting bacterial strains of the species Pseudomonas aeruginosa.

18. A method for genotyping and pathotyping Pseudomonas aeruginosa, comprising the following steps:
a) contacting the sample with a nucleic acid chip in a microarray device according to claim 2; and
b) detecting the interaction between the oligonucleotide probes and the target nucleic acids contained in the sample.

19. A method for amplifying nucleic acids of bacterial strains of the species Pseudomonas aeruginosa, comprising the following steps:
a) contacting the sample with a nucleic acid chip in a microarray device according to claim 2; and
b) detecting the interaction between the oligonucleotide probes and the target nucleic acids contained in the sample.
